SEQUENCE LISTING (227 sequences)

SEQ ID NO: 1 - DNA, 524 bp, Brassica napus
  misc_feature (1)..(524): ceres Seq. ID no. 13507036
attatcatcatcacatccctctcttcttcactacaacacacaaaaatggagaaaccatcctcaatgaaacaatggaagaaaggtccagctcgtggcaaaggcggtccacaaaacgctctttgccgttaccgtggagtgaggcaaagaacatggggcaaatgggtggctgagatcagagagcccaagaagagagcaagactctggctcggctctttcgctacagctgaagaagcagccatggcttatgacgaagccgcgctgaaactatatggtcctgaagcttatctcaacttacctcatcttcagcagaggaaacaacaaggacctcctctgaacaacactcagaggtttaaatgggttccttcaaagaagtttatctccatgtttccttctcgcggtttgctgaatgtgaatgctcagcctagtgttcaagttatccagcagagacttgatgagctcaagagaaacgggcttctatctcagtcctattcttctagctcttcctccacggagtcgaaaagcct

SEQ ID NO: 2 - protein, 174 aa, Brassica napus
  peptide (1)..(174): ceres Seq. ID no. 13507037
IIIITSLSSSLQHTKMEKPSSMKQWKKGPARGKGGPQNALCRYRGVRQRTWGKWVAEIREPKKRARLWLGSFATAEEAAMAYDEAALKLYGPEAYLNLPHLQQRKQQGPPLNNTQRFKWVPSKKFISMFPSRGLLNVNAQPSVQVIQQRLDELKRNGLLSQSYSSSSSSTESKS

SEQ ID NO: 3 - protein, 159 aa, Brassica napus
  peptide (1)..(159): ceres Seq. ID no. 13507038
MEKPSSMKQWKKGPARGKGGPQNALCRYRGVRQRTWGKWVAEIREPKKRARLWLGSFATAEEAAMAYDEAALKLYGPEAYLNLPHLQQRKQQGPPLNNTQRFKWVPSKKFISMFPSRGLLNVNAQPSVQVIQQRLDELKRNGLLSQSYSSSSSSTESKS

SEQ ID NO: 4 - protein, 153 aa, Brassica napus
  peptide (1)..(153): ceres Seq. ID no. 13507039
MKQWKKGPARGKGGPQNALCRYRGVRQRTWGKWVAEIREPKKRARLWLGSFATAEEAAMAYDEAALKLYGPEAYLNLPHLQQRKQQGPPLNNTQRFKWVPSKKFISMFPSRGLLNVNAQPSVQVIQQRLDELKRNGLLSQSYSSSSSSTESKS

SEQ ID NO: 5 - DNA, 404 bp, Brassica napus
  misc_feature (1)..(404): ceres Seq. ID no. 4450880
aagacaatcttctctttctccactctcattctctttctcactctgacgcatcaactacttatttttcctgcattgctttctctctatctctatctctcacacacgcatacacacaaagacatttaacatatatcaagaaagaagatgtcgagcagaagatcatcatgttcaagacagtccgggagctcaagaatctctgacgatcaaatttccgatctagttaccaagcttcaacacctcatccctgaactccgccgccgccgttctgacmaggtgtcagcatctaaggtactacaagagacttgcaactacatcaggaacttacacagagaggttgatgatctcagtgaccgattgtccgaatttttggcttccacggacgacaacagcgccgaarcagccat

SEQ ID NO: 6 - protein, 86 aa, Brassica napus
  misc_feature (43)..(43): Xaa can be any naturally occurring amino acid
  misc_feature (85)..(85): Xaa can be any naturally occurring amino acid
  peptide (1)..(86): ceres Seq. ID no. 4450881
MSSRRSSCSRQSGSSRISDDQISDLVTKLQHLIPELRRRRSDXVSASKVLQETCNYIRNLHREVDDLSDRLSEFLASTDDNSAEXA

SEQ ID NO: 7 - protein, 65 aa, Brassica napus
  peptide (1)..(65): ceres Seq. ID no. 4450882
DNLLFLHSHSLSHSDASTTYFSCIAFSLSLSLTHAYTQRHLTYIKKEDVEQKIIMFKTVRELKNL

SEQ ID NO: 8 - DNA, 520 bp, Brassica napus
  misc_feature (502)..(502): n is a, c, g, or t
  misc_feature (1)..(520): ceres Seq. ID no. 13507815
aaaaccctaacatccgtccccactccccaatcaacctgccgccgcccgccagcaccaaacttccatccgccccgagctctgcctccgccgccgccgcgccatgaggcctctcgacgagaaggagacgactcaggtattcgagaagctgttcaagttcacggggcccaacctgaagcacctcctggagcgccccgcagtggagggccccgacgcggagcccggccgctactgcctccggcttcaccgcaatcgcgtctactacgcctccgaggcgctcgtgcgccgcgccaccgccgtgtcccgaccgcgcctcgccggcgtggggacgcccatcggcaagttcacccaccacggcgcgttccaccttacggtccacgcgctcgacctcctcgctgtacacgcgcgccgscgcgtctggctcaaacccgacaccgagcgttcctttctctttggcaattccgtgcccaagtcttctctcgctcgcatyaccgagaacaccaangccggggacggggtggtc

SEQ ID NO: 9 - protein, 173 aa, Brassica napus
  misc_feature (137)..(137): Xaa can be any naturally occurring amino acid
  misc_feature (163)..(163): Xaa can be any naturally occurring amino acid
  misc_feature (168)..(168): Xaa can be any naturally occurring amino acid
  peptide (1)..(173): ceres Seq. ID no. 13507816
KTLTSVPTPQSTCRRPPAPNFHPPRALPPPPPRHEASRREGDDSGIREAVQVHGAQPEAPPGAPRSGGPRRGARPLLPPASPQSRLLRLRGARAPRHRRVPTAPRRRGDAHRQVHPPRRVPPYGPRARPPRCTRAPXRLAQTRHRAFLSLWQFRAQVFSRSHXREHQXRGRGG

SEQ ID NO: 10 - protein, 140 aa, Brassica napus
  misc_feature (103)..(103): Xaa can be any naturally occurring amino acid
  misc_feature (129)..(129): Xaa can be any naturally occurring amino acid
  misc_feature (134)..(134): Xaa can be any naturally occurring amino acid
  peptide (1)..(140): ceres Seq. ID no. 13507817
MRPLDEKETTQVFEKLFKFTGPNLKHLLERPAVEGPDAEPGRYCLRLHRNRVYYASEALVRRATAVSRPRLAGVGTPIGKFTHHGAFHLTVHALDLLAVHARXRVWLKPDTERSFLFGNSVPKSSLARXTENTXAGDGVV

SEQ ID NO: 11 - DNA, 451 bp, Brassica napus
  misc_feature (1)..(451): ceres Seq. ID no. 4435719
aaatcagtgacaattcctaccttgcgccggtggtttgcggctcttcgaaaatgggtgcgtacaagtatgtgtctgagctatggaggaagaaacaatccgatgtgatgaggtttgtgcagagggtgaggtgctgggagtacagacagcagccttccattgtccgtctcgtcaggcctactcgtcccgacaaggctcgccgtttgggttacaaggccaagcagggctttgtggtgtaccgtgtacgtgtgagacgtggtggaagaaagaggccagtcccaaagggtattgtgtatggtaaacccacaaaccagggagttactcaactcaagttccagaggagcaagcgttctgttgctgaggagcgtgctggaaggaaactgggcggtctaagagtcgtcaactcctactggctcaacgaggattcgacgtacaagtactacgagatcatctt

SEQ ID NO: 12 - protein, 149 aa, Brassica napus
  peptide (1)..(149): ceres Seq. ID no. 4435720
ISDNSYLAPVVCGSSKMGAYKYVSELWRKKQSDVMRFVQRVRCWEYRQQPSIVRLVRPTRPDKARRLGYKAKQGFVVYRVRVRRGGRKRPVPKGIVYGKPTNQGVTQLKFQRSKRSVAEERAGRKLGGLRVVNSYWLNEDSTYKYYEII

SEQ ID NO: 13 - protein, 133 aa, Brassica napus
  peptide (1)..(133): ceres Seq. ID no. 4435721
MGAYKYVSELWRKKQSDVMRFVQRVRCWEYRQQPSIVRLVRPTRPDKARRLGYKAKQGFVVYRVRVRRGGRKRPVPKGIVYGKPTNQGVTQLKFQRSKRSVAEERAGRKLGGLRVVNSYWLNEDSTYKYYEII

SEQ ID NO: 14 - protein, 115 aa, Brassica napus
  peptide (1)..(115): ceres Seq. ID no. 4435722
MRFVQRVRCWEYRQQPSIVRLVRPTRPDKARRLGYKAKQGFVVYRVRVRRGGRKRPVPKGIVYGKPTNQGVTQLKFQRSKRSVAEERAGRKLGGLRVVNSYWLNEDSTYKYYEII

SEQ ID NO: 15 - DNA, 497 bp, Brassica napus
  misc_feature (484)..(484): n is a, c, g, or t
  misc_feature (1)..(497): ceres Seq. ID no. 12474809
gtcggccacattatgtttggtactaaattcctctaaaaccctaaaaacagagagcatccgtcttcccaaaaaaaaagatgcggccgttagacgagaacgagacgacagtggtcttcgagaagatcttcaaattcgtcggaaacaacctcaagaacatcgtcgagaacccatcccacgaaggacccggacccaaacccgaagccgggcgctactgcttccgcctccagaagagcagagtctactacgtaagcgagtctctcgtgaagcgagccaccaacatctcccgcaaaaacctagtctccctcggaacctgcatcgggaagtacacccacgcggggagcttccaccttacgatcatgtcgctcaacctcttggccgcgaacgcgaagcacaaggtttggcttaaacccacttcggagatgtcgtttctttacgggaaccatgtcttgaaaggagggctagggaggattaccgatagtattgngcctggagatggg

SEQ ID NO: 16 - protein, 140 aa, Brassica napus
  misc_feature (136)..(136): Xaa can be any naturally occurring amino acid
  peptide (1)..(140): ceres Seq. ID no. 12474810
MRPLDENETTVVFEKIFKFVGNNLKNIVENPSHEGPGPKPEAGRYCFRLQKSRVYYVSESLVKRATNISRKNLVSLGTCIGKYTHAGSFHLTIMSLNLLAANAKHKVWLKPTSEMSFLYGNHVLKGGLGRITDSIXPGDG

SEQ ID NO: 17 - protein, 134 aa, Brassica napus
  peptide (1)..(134): ceres Seq. ID no. 12474811
SATLCLVLNSSKTLKTESIRLPKKKDAAVRRERDDSGLREDLQIRRKQPQEHRREPIPRRTRTQTRSRALLLPPPEEQSLLRKRVSREASHQHLPQKPSLPRNLHREVHPRGELPPYDHVAQPLGREREAQGLA

SEQ ID NO: 18 - DNA, 814 bp, Brassica napus
  misc_feature (802)..(802): n is a, c, g, or t
  misc_feature (808)..(808): n is a, c, g, or t
  misc_feature (1)..(814): ceres Seq. ID no. 13504237
gaagcttttttttatctctctctccgccgtcactctcacctgcggcgaggcttaacgatctcagccatggccgccgccgtagaaatcgacgcagagatccagcagcagcttaccaacgaggtcaagctcttcaaccgctggacctacgatgacgtctcggttgcagatatcagtcttgttgactacattggagttcaagcagccaaacacgccacctttgtcccccacactgcaggaagatactctgtgaagagattcaggaaggctcagtgcccaattgttgagaggctcacaaactctctcatgatgcacggaagaaacaatggaaagaagttgatggcggtgaggatcatcaagcacgccatggagatcatccacctcttgactgacgccaacccgattcagatcatcatcgatgccattgtcaacagtggtccacgtgaagatgccaccagaattggatctgctggtgtcgttaggagacaagctgttgatatctctcctttgagacgtgttaaccaggcgatcttcttgctcacaaccggtgctcgtgaggctgctttcagaaacattaagactattgctgagtgccttgctgatgagttaatcaacgcagccaagggctcgtccaacagctatgccatcaaaaagaaggatgagattgagagagttgccaaggctaatcgttaagggaaagtcttcttttaccctgatggttgttacaatcttatcaatgaaatttatgttttgtttcgactaagattgggaatatgtaacagatgttttgctcttttcgtcttangaaagnttattt

SEQ ID NO: 19 - protein, 229 aa, Brassica napus
  peptide (1)..(229): ceres Seq. ID no. 13504238
EAFFYLSLRRHSHLRRGLTISAMAAAVEIDAEIQQQLTNEVKLFNRWTYDDVSVADISLVDYIGVQAAKHATFVPHTAGRYSVKRFRKAQCPIVERLTNSLMMHGRNNGKKLMAVRIIKHAMEIIHLLTDANPIQIIIDAIVNSGPREDATRIGSAGVVRRQAVDISPLRRVNQAIFLLTTGAREAAFRNIKTIAECLADELINAAKGSSNSYAIKKKDEIERVAKANR

SEQ ID NO: 20 - protein, 207 aa, Brassica napus
  peptide (1)..(207): ceres Seq. ID no. 13504239
MAAAVEIDAEIQQQLTNEVKLFNRWTYDDVSVADISLVDYIGVQAAKHATFVPHTAGRYSVKRFRKAQCPIVERLTNSLMMHGRNNGKKLMAVRIIKHAMEIIHLLTDANPIQIIIDAIVNSGPREDATRIGSAGVVRRQAVDISPLRRVNQAIFLLTTGAREAAFRNIKTIAECLADELINAAKGSSNSYAIKKKDEIERVAKANR

SEQ ID NO: 21 - protein, 128 aa, Brassica napus
  peptide (1)..(128): ceres Seq. ID no. 13504240
MMHGRNNGKKLMAVRIIKHAMEIIHLLTDANPIQIIIDAIVNSGPREDATRIGSAGVVRRQAVDISPLRRVNQAIFLLTTGAREAAFRNIKTIAECLADELINAAKGSSNSYAIKKKDEIERVAKANR

SEQ ID NO: 22 - DNA, 724 bp, Brassica napus
  misc_feature (1)..(724): ceres Seq. ID no. 12472572
ataaatttcgatcgatgtctacgttcttaacacaattcacaatcttctttagaatccaaaatcattgttcgaagaagaaaaaaccgaagatggcaacaacatccacgggagatatcgagaaaacgaagtcagtagaagtgaagaagaaacttaacgtgttgatcgtcgatgatgatcctgtaattcgtaaacttcacgagattatcatcaaatcaatcggtggaatttcacagacagctaagaacggtgaggaggcagtgaacatccaccgcgacggcaatgcatctttcgaccttatcctaatggataaagaaatgcccgagagggatggactttcggcaactaagaagctaagagaaatgaaagtgacgtctatgattattggggtgacgacactggctgacaatgaagaggaacgtaaggctttcatggaagctggacttaaccattgcttggcaaagcccttaagcaaagccaagatcctccctctcatcaacaatctcatggatgcttgatggatggatgaattgtcgccactacatatctacattatataaatatgaaaaacacataataacgtcagcttatacacctgtgtgtgtatgcatatatctatctgcatgtgtgtgttttagggttgttatgtttgatttttatcgtgcgtggcgtgatatacaatcatgtaagtcattacttttggcttataaaataatgaataagattt

SEQ ID NO: 23 - protein, 170 aa, Brassica napus
  peptide (1)..(170): ceres Seq. ID no. 12472573
KFRSMSTFLTQFTIFFRIQNHCSKKKKPKMATTSTGDIEKTKSVEVKKKLNVLIVDDDPVIRKLHEIIIKSIGGISQTAKNGEEAVNIHRDGNASFDLILMDKEMPERDGLSATKKLREMKVTSMIIGVTTLADNEEERKAFMEAGLNHCLAKPLSKAKILPLINNLMDA

SEQ ID NO: 24 - protein, 166 aa, Brassica napus
  peptide (1)..(166): ceres Seq. ID no. 12472574
MSTFLTQFTIFFRIQNHCSKKKKPKMATTSTGDIEKTKSVEVKKKLNVLIVDDDPVIRKLHEIIIKSIGGISQTAKNGEEAVNIHRDGNASFDLILMDKEMPERDGLSATKKLREMKVTSMIIGVTTLADNEEERKAFMEAGLNHCLAKPLSKAKILPLINNLMDA

SEQ ID NO: 25 - protein, 141 aa, Brassica napus
  peptide (1)..(141): ceres Seq. ID no. 12472575
MATTSTGDIEKTKSVEVKKKLNVLIVDDDPVIRKLHEIIIKSIGGISQTAKNGEEAVNIHRDGNASFDLILMDKEMPERDGLSATKKLREMKVTSMIIGVTTLADNEEERKAFMEAGLNHCLAKPLSKAKILPLINNLMDA

SEQ ID NO: 26 - DNA, 568 bp, Brassica napus
  misc_feature (1)..(568): ceres Seq. ID no. 13508521
gatttgatcattttcaggttcgctttgctcttctccctcgctgcagattctgttcgttacttctctcaattccttaattgtaatctgttcggatgagaattagtcgttgagcgttgtttctgtgtggtgttttctctctgaatccattgtgctttcgcattagatctctcattgccgtaatcaattttcttagagtgttaagttgttagttacaatcaattttttacattagcttgtttgggggtgttttggtctttgttaaaagatcacagcaaataaaaatgggagctacgaagctttccgggatgcaaaagcaagtcctgagtctctacagaggattcctaagagcagctcgttcaaagccaacgacggaagacaggaagagaatagagacggtcgtgtcgacagagtttcgcaagaactcaaaggaggttgaccgtaagaactttcagtacattgagtatctgcttcggctagctcataaacaacttgaccaactcaaaagtcccgatatggkttccatctcctctgtgaagatcaactagtctttatatctctcaaggkgatc

SEQ ID NO: 27 - protein, 87 aa, Brassica napus
  misc_feature (79)..(79): Xaa can be any naturally occurring amino acid
  peptide (1)..(87): ceres Seq. ID no. 13508522
MGATKLSGMQKQVLSLYRGFLRAARSKPTTEDRKRIETVVSTEFRKNSKEVDRKNFQYIEYLLRLAHKQLDQLKSPDMXSISSVKIN

SEQ ID NO: 28 - protein, 79 aa, Brassica napus
  misc_feature (71)..(71): Xaa can be any naturally occurring amino acid
  peptide (1)..(79): ceres Seq. ID no. 13508523
MQKQVLSLYRGFLRAARSKPTTEDRKRIETVVSTEFRKNSKEVDRKNFQYIEYLLRLAHKQLDQLKSPDMXSISSVKIN

SEQ ID NO: 29 - DNA, 432 bp, Brassica napus
  misc_feature (1)..(432): ceres Seq. ID no. 4625303
attgcttctctgaatttctatattcgttaatcagaagccgttttgatcattttcaggtgttttgactttgttaaagataacaacagcaaataaaaatgggagctacgaaactttcggggatgcaaaagcaagtcctgagtctctacagaggattcctaagagcagctcgttcaacggaagacaggaagagaatacagacgatcgtgtcgacagagtttcgcaagaactcaaaggaggttgaccgtaagaactttcagtacatcgagtatctgcttcggctagctcataaacaacttgaccaactcaaaagtcccgatatggtttccatctcctctgtgaagatcaactagtctttatatctctcaaggtgatcatatttgttatgtagaatcatcatagagaatcaaagagaggcaaactgattcctttaat

SEQ ID NO: 30 - protein, 84 aa, Brassica napus
  peptide (1)..(84): ceres Seq. ID no. 4625304
MGATKLSGMQKQVLSLYRGFLRAARSTEDRKRIQTIVSTEFRKNSKEVDRKNFQYIEYLLRLAHKQLDQLKSPDMVSISSVKIN

SEQ ID NO: 31 - protein, 76 aa, Brassica napus
  peptide (1)..(76): ceres Seq. ID no. 4625305
MQKQVLSLYRGFLRAARSTEDRKRIQTIVSTEFRKNSKEVDRKNFQYIEYLLRLAHKQLDQLKSPDMVSISSVKIN

SEQ ID NO: 32 - DNA, 634 bp, Brassica napus
  misc_feature (1)..(634): ceres Seq. ID no. 13503679
attcacaatcttctttagaatccaaattattgttctaagacagaatcgaagatggcaacaaaatccatgggagatatcgagaaaataaagaagaaactaaacgtgttgatcgtcgatgatgatccactaaaccttataattcatgagaagatcatcaaagcgattgggggtatttcacagacagcgaataacggtgaggaggcagtaatcatccaccgtgacggcggctcatcttttgaccttatcctaatggataaagaaatgcccgagagggatggtgtttcgacaactaagaagctaagagaaatggaagtgaagtcaatgattgttggggtgacttcactggctgacaatgaagaggagcgcagggctttcatggaagctggacttaaccattgcttggcaaaaccgttaaccaaggacaagatcatccctctcattaaccaactcatggatgcttgatggatatatattttatattatggaaacacacataataacgtctaagtgtgtatgtatgcatagatacttgcatgtgtgtgttttagaatttagggttctttatcgtccgtgatatataatcatgtaagttgttgctttaagcttataaaatatttaaataagggtttcctct

SEQ ID NO: 33 - protein, 136 aa, Brassica napus
  peptide (1)..(136): ceres Seq. ID no. 13503680
MATKSMGDIEKIKKKLNVLIVDDDPLNLIIHEKIIKAIGGISQTANNGEEAVIIHRDGGSSFDLILMDKEMPERDGVSTTKKLREMEVKSMIVGVTSLADNEEERRAFMEAGLNHCLAKPLTKDKIIPLINQLMDA

SEQ ID NO: 34 - protein, 131 aa, Brassica napus
  peptide (1)..(131): ceres Seq. ID no. 13503681
MGDIEKIKKKLNVLIVDDDPLNLIIHEKIIKAIGGISQTANNGEEAVIIHRDGGSSFDLILMDKEMPERDGVSTTKKLREMEVKSMIVGVTSLADNEEERRAFMEAGLNHCLAKPLTKDKIIPLINQLMDA

SEQ ID NO: 35 - protein, 70 aa, Brassica napus
  peptide (1)..(70): ceres Seq. ID no. 13503682
MDKEMPERDGVSTTKKLREMEVKSMIVGVTSLADNEEERRAFMEAGLNHCLAKPLTKDKIIPLINQLMDA

SEQ ID NO: 36 - DNA, 475 bp, Brassica napus
  misc_feature (1)..(475): ceres Seq. ID no. 4794495
aaaacttycttctttccttttttcttttcctgttgtcttgtattccgaaagctgaattgagttgggtgaagcaattctcaggatgggaagaggaaagatagaaatcaagaggatagagaattcaacaaatcgacaagtgacgttctgcaaaagaagaaatggactcctgaagaaggcttacgagctttcagttctctgtgatgccgaggttgccctcatcgtcttctcaactcgtggccgtctctatgagtacgccaataacaacataagatcaaccattgagaggtacaagaaggcttctgataacaccaacactcactctgtacaagagattaatgcagcgtactatcaacaagaatctgcaaagctgaggcaacagatccaaacgattcaaaattccaacaggcatctgatgggagactctttgagtgccttaagtgtcaaggagctaaaacaggttgagaatcgccttgag

SEQ ID NO: 37 - protein, 131 aa, Brassica napus
  peptide (1)..(131): ceres Seq. ID no. 4794496
MGRGKIEIKRIENSTNRQVTFCKRRNGLLKKAYELSVLCDAEVALIVFSTRGRLYEYANNNIRSTIERYKKASDNTNTHSVQEINAAYYQQESAKLRQQIQTIQNSNRHLMGDSLSALSVKELKQVENRLE

SEQ ID NO: 38 - DNA, 452 bp, Brassica napus
  misc_feature (1)..(452): ceres Seq. ID no. 6426831
ctaagcagcaatggatcttctcttgttggaaaaatctctgatcgccgtctttgcggcggtggttctcgccaccgtgatctccaagctccgcggsgggaaactaaacctacctcctggtcctattcccattcccatcttcggaaactggctacaagtgggagatgatctcaaccaccgtaacctcgtcgactacgccaagaagttcggagacctcttcctcctccgtatgggccaacgaaacctagtcgtcgtctcttcaccaaacctaaccaaagaagtgctccacacgcaaggcgtagagttcggatctcggacaagaaacgtcgtcttcgacatcttcacaggcaaaggacaggacatggtgttcacagtgtacggcgaacactggcgtaagatgcgtcggatcatgacggttccgtttttcaccaacaaggttgtgcaacggaacaggg

SEQ ID NO: 39 - protein, 147 aa, Brassica napus
  misc_feature (28)..(28): Xaa can be any naturally occurring amino acid
  peptide (1)..(147): ceres Seq. ID no. 6426832
MDLLLLEKSLIAVFAAVVLATVISKLRXGKLNLPPGPIPIPIFGNWLQVGDDLNHRNLVDYAKKFGDLFLLRMGQRNLVVVSSPNLTKEVLHTQGVEFGSRTRNVVFDIFTGKGQDMVFTVYGEHWRKMRRIMTVPFFTNKVVQRNR

SEQ ID NO: 40 - protein, 75 aa, Brassica napus
  peptide (1)..(75): ceres Seq. ID no. 6426833
MGQRNLVVVSSPNLTKEVLHTQGVEFGSRTRNVVFDIFTGKGQDMVFTVYGEHWRKMRRIMTVPFFTNKVVQRNR

SEQ ID NO: 41 - DNA, 426 bp, Brassica napus
  misc_feature (1)..(426): ceres Seq. ID no. 6425768
gagaggattcgagtttttctatagtctagacaacactcgagctccgtttcgtttgacaccgagattgcactcagcgccgtcccgtgtcacaaaccctccgacgagctctccggcagccatgtacggcggagatgaagtgtcagctatagtggttgacttgggttcgcacacttgcaaggctggctacgccggcgaggatgctcccaaggccgtcttcccttccgttgttggggcagtggatggagtagaggcaatggaagtggatgttgattctgcaaagactaattcgaattcagaggataagtctgaaaaggacaagggtaaacgcaagctctgtgtgggatctcaagccttgaattaccgcagggatcatatggagatcttgtcaccgatcaaagatggcatagtttctgattgggatttggt

SEQ ID NO: 42 - protein, 141 aa, Brassica napus
  peptide (1)..(141): ceres Seq. ID no. 6425769
RGFEFFYSLDNTRAPFRLTPRLHSAPSRVTNPPTSSPAAMYGGDEVSAIVVDLGSHTCKAGYAGEDAPKAVFPSVVGAVDGVEAMEVDVDSAKTNSNSEDKSEKDKGKRKLCVGSQALNYRRDHMEILSPIKDGIVSDWDL

SEQ ID NO: 43 - protein, 102 aa, Brassica napus
  peptide (1)..(102): ceres Seq. ID no. 6425770
MYGGDEVSAIVVDLGSHTCKAGYAGEDAPKAVFPSVVGAVDGVEAMEVDVDSAKTNSNSEDKSEKDKGKRKLCVGSQALNYRRDHMEILSPIKDGIVSDWDL

SEQ ID NO: 44 - DNA, 481 bp, Brassica napus
  misc_feature (1)..(481): ceres Seq. ID no. 6425497
caaacacttatcttcaaatcctgatcaaatctttgtcttgtaaacacagaaagttcttaagcaatggaagctacttcacagccttgtttctctcataattaccgtagatcatctgaaaactacaactcatcgctccattcgacacgaaagcaaccagcaaagccatggaaaagacccgtaacagcttcgcttcaacgaatgcatcccagagtttaccgagtcgagcctgtgaacttcaaagagttggttcagagtctaaccggcgcaccacaagatcatgagagagatcaggtgcatcaagttgaaactaagccactactcaagatgcaacacggtctagttgaggttaggcagccacttaagattttccatgaaacaacaactcaagagaacccagtggcgtttgatctgtctccttcatcatctcgtttttgggaagctttccctcttctcagtcctgcgaatctatcaagatggtagg

SEQ ID NO: 45 - protein, 138 aa, Brassica napus
  peptide (1)..(138): ceres Seq. ID no. 6425498
MEATSQPCFSHNYRRSSENYNSSLHSTRKQPAKPWKRPVTASLQRMHPRVYRVEPVNFKELVQSLTGAPQDHERDQVHQVETKPLLKMQHGLVEVRQPLKIFHETTTQENPVAFDLSPSSSRFWEAFPLLSPANLSRW

SEQ ID NO: 46 - protein, 93 aa, Brassica napus
  peptide (1)..(93): ceres Seq. ID no. 6425499
MHPRVYRVEPVNFKELVQSLTGAPQDHERDQVHQVETKPLLKMQHGLVEVRQPLKIFHETTTQENPVAFDLSPSSSRFWEAFPLLSPANLSRW

SEQ ID NO: 47 - DNA, 527 bp, Brassica napus
  misc_feature (504)..(504): n is a, c, g, or t
  misc_feature (1)..(527): ceres Seq. ID no. 6416837
atatcagtcacaccaccaacaaagtagctccctttttcttcaatcactaactcctccgtctcaagaagcgaagaacaaagttacatcttcttgttctccttcctctggtgctcctgcttattctttcatggagatcaatcaccaaaacgaactcctcgcaggaggactcaatccctgttcttcagcctctgtcaaggccaaaatcatgggtcatcctcactaccaccgcctcttactcgcctatgtcaattgccagaaggtgggagctccaccggaagtgcaggcgaggctggaagaaacatgctcgtctgcggcggcagccgcagcgtcgatgggacccacaggttctttaggtgaagatccagggcttgatcagttcatggaagcgtactgtgaaatgctcgttaagtacgagcaagaactctctaaaccttttaaagaagctatggtcttccttcaacacgtcgagtgtcaattcaaatccctctctctctcctcgccttnctccttctctggttatggagagg

SEQ ID NO: 48 - protein, 175 aa, Brassica napus
  misc_feature (168)..(168): Xaa can be any naturally occurring amino acid
  peptide (1)..(175): ceres Seq. ID no. 6416838
YQSHHQQSSSLFLQSLTPPSQEAKNKVTSSCSPSSGAPAYSFMEINHQNELLAGGLNPCSSASVKAKIMGHPHYHRLLLAYVNCQKVGAPPEVQARLEETCSSAAAAAASMGPTGSLGEDPGLDQFMEAYCEMLVKYEQELSKPFKEAMVFLQHVECQFKSLSLSSPXSFSGYGE

SEQ ID NO: 49 - protein, 133 aa, Brassica napus
  misc_feature (126)..(126): Xaa can be any naturally occurring amino acid
  peptide (1)..(133): ceres Seq. ID no. 6416839
MEINHQNELLAGGLNPCSSASVKAKIMGHPHYHRLLLAYVNCQKVGAPPEVQARLEETCSSAAAAAASMGPTGSLGEDPGLDQFMEAYCEMLVKYEQELSKPFKEAMVFLQHVECQFKSLSLSSPXSFSGYGE

SEQ ID NO: 50 - protein, 107 aa, Brassica napus
  misc_feature (100)..(100): Xaa can be any naturally occurring amino acid
  peptide (1)..(107): ceres Seq. ID no. 6416840
MGHPHYHRLLLAYVNCQKVGAPPEVQARLEETCSSAAAAAASMGPTGSLGEDPGLDQFMEAYCEMLVKYEQELSKPFKEAMVFLQHVECQFKSLSLSSPXSFSGYGE

SEQ ID NO: 51 - DNA, 557 bp, Arabidopsis thaliana
  misc_feature (1)..(557): ceres Seq. ID no. 12330042
atactatcaacttttctctatctatctctctctcttctttttccggcataacttctgtgttaccctaaactccataacctgtttcatcgataaagtgcctttgcttctatctctgtcactcttactacttgttgaacaatattctacaaaaaaatgtcgggaagaagatcacgttcgaggcaatcatcaggaacttcaaggatctcagaagatcaaatcaatgatctgattatcaagttgcaacagcttcttcctgagctcagggacagtcgtcgttccgacaaggtttcagcagcgagggtgttacaagatacgtgcaactacatacggaatctgcatagagaggttgatgatctaagtgagaggctatctgagttactagcaaactcagacactgcacaagctgctttaatcagaagcttacttacccaataattcctatctatctttttcttcttcttcttttttttgtttactataataataataatagtttgcgggtttttttttctatagatgttgatgaccttataaacgtttaatgatacgagttcgtc

SEQ ID NO: 52 - protein, 93 aa, Arabidopsis thaliana
  peptide (1)..(93): ceres Seq. ID no. 12330043
MSGRRSRSRQSSGTSRISEDQINDLIIKLQQLLPELRDSRRSDKVSAARVLQDTCNYIRNLHREVDDLSERLSELLANSDTAQAALIRSLLTQ

SEQ ID NO: 53 - protein, 75 aa, Arabidopsis thaliana
  peptide (1)..(75): ceres Seq. ID no. 12330044
YYQLFSIYLSLFFFRHNFCVTLNSITCFIDKVPLLLSLSLLLLVEQYSTKKCREEDHVRGNHQELQGSQKIKSMI

SEQ ID NO: 54 - DNA, 911 bp, Arabidopsis thaliana
  misc_feature (1)..(911): ceres Seq. ID no. 12420894
ctcttaaagcttctcgttttctctgccgtctctctcattcgcgcgacgcaaacgatcttcagccatggccaccgccgcagatgttgacgctgagattcagcaggcgctcactaacgaagtcaagctcttcaaccgctggacctatgacgacgttacggtcacagacatcagtcttgttgactacattggagttcaggcagctaaacatgctacctttgttccccacaccgctggaagatactctgtgaagagattcaggaaggctcagtgccccattgttgagaggctcaccaactctctcatgatgcacgggaggaacaacggtaagaaattgatggctgtcaggatcgtcaagcacgccatggagattatccacctcttgtctgacttgaacccaattcaggtcatcattgacgccattgtcaacagtggtccacgtgaagatgctaccagaattggatctgctggtgttgttaggagacaagctgttgatatctctcctctaagacgtgttaaccaggctatcttcttgattaccactggtgctcgtgaagctgctttcagaaacatcaagactatagctgagtgccttgctgatgaattgatcaacgcagccaagggctcttccaacagctatgccatcaagaagaaggatgagattgaaagagttgccaaggctaatcgttaaggattttccctttgccctgtgcgttacaatctcgtatcaatgagtttaatgttttatcttcatttagattgaaatatgtatctcagatgtttgctcttttgttttatgaagtttatttcgtctgaactactttgaatagataaatttttgatgctttaagctgtttcgcatcagtttattgtcaatttttgaacttatctaggccacctgaagctagaaattt

SEQ ID NO: 55 - protein, 207 aa, Arabidopsis thaliana [sequence omitted]

SEQ ID NO: 56 - protein, 128 aa, Arabidopsis thaliana
  peptide (1)..(128): ceres Seq. ID no. 12420896
MMHGRNNGKKLMAVRIVKHAMEIIHLLSDLNPIQVIIDAIVNSGPREDATRIGSAGVVRRQAVDISPLRRVNQAIFLITTGAREAAFRNIKTIAECLADELINAAKGSSNSYAIKKKDEIERVAKANR

SEQ ID NO: 57 - protein, 127 aa, Arabidopsis thaliana
  peptide (1)..(127): ceres Seq. ID no. 12420897
MHGRNNGKKLMAVRIVKHAMEIIHLLSDLNPIQVIIDAIVNSGPREDATRIGSAGVVRRQAVDISPLRRVNQAIFLITTGAREAAFRNIKTIAECLADELINAAKGSSNSYAIKKKDEIERVAKANR

SEQ ID NO: 58 - DNA, 1198 bp, Arabidopsis thaliana
  misc_feature (1)..(1198): ceres Seq. ID no. 13491860
atatcagtcacaccaccaacaaagtagttccctttttcttcaatcactagctcctccccaaggaactaagaacaaagttgcttcttcttcttctccttcctcttgtgctcctgcctattctctaatggagatccatcataacgaaatcgttgcaggaggaatcaacccttgctcctcttcctcttcttcagcctctgtcaaggccaagatcatggctcatcctcactaccaccgcctcttggccgcttatgtcaattgtcagaaggttggagcaccaccggaggttgtggcgaggctagaggaggcatgctcgtctgccgcagccgctgccgcatctatgggaccaacaggatgtctaggtgaagatccagggcttgatcaattcatggaagcttactgtgaaatgctcgttaagtatgagcaagagctctccaaacctttcaaggaagctatggtcttccttcaacgtgtcgagtgtcaattcaaatccctctctctatcctcaccttcctctttctccggttatggagagacagcaattgataggaacaataatgggtcatccgaggaagaagtcgatatgaacaatgaatttgtagatccacaagctgaggatagagagcttaaaggacagctcttgcgcaagtacagtggttacttagggagcctcaagcaagagttcatgaagaagaggaagaaaggaaagctccctaaagaagctcgtcaacaactgcttgattggtggagccgtcactacaaatggccttacccttcggagcaacaaaagctcgcccttgcggaatcaacggggctggaccagaaacagataaacaattggttcataaaccagaggaaacggcattggaagccgtcggaggacatgcagtttgtagtaatggacgcaacacatcctcaccattacttcatggataatgtcttgggcaatcctttcccaatggatcacatctcctccaccatgctttgatcgatcctttaaaacgtttatttcattatatttataatcgttaaacagcttataatatgtataatctatggttctcggtatacttggaacatatgaaacatgttccgattgatgaacaagcgtttgatgttgtggtgtgtatccttttgtgttgttctattgttatgcgaaattcctattcctattataatgatagtaaagcttttttgatct

SEQ ID NO: 59 - protein, 327 aa, Arabidopsis thaliana
  peptide (1)..(327): ceres Seq. ID no. 13491861
YQSHHQQSSSLFLQSLAPPQGTKNKVASSSSPSSCAPAYSLMEIHHNEIVAGGINPCSSSSSSASVKAKIMAHPHYHRLLAAYVNCQKVGAPPEVVARLEEACSSAAAAAASMGPTGCLGEDPGLDQFMEAYCEMLVKYEQELSKPFKEAMVFLQRVECQFKSLSLSSPSSFSGYGETAIDRNNNGSSEEEVDMNNEFVDPQAEDRELKGQLLRKYSGYLGSLKQEFMKKRKKGKLPKEARQQLLDWWSRHYKWPYPSEQQKLALAESTGLDQKQINNWFINQRKRHWKPSEDMQFVVMDATHPHHYFMDNVLGNPFPMDHISSTML

SEQ ID NO: 60 - protein, 286 aa, Arabidopsis thaliana
  peptide (1)..(286): ceres Seq. ID no. 13491862
MEIHHNEIVAGGINPCSSSSSSASVKAKIMAHPHYHRLLAAYVNCQKVGAPPEVVARLEEACSSAAAAAASMGPTGCLGEDPGLDQFMEAYCEMLVKYEQELSKPFKEAMVFLQRVECQFKSLSLSSPSSFSGYGETAIDRNNNGSSEEEVDMNNEFVDPQAEDRELKGQLLRKYSGYLGSLKQEFMKKRKKGKLPKEARQQLLDWWSRHYKWPYPSEQQKLALAESTGLDQKQINNWFINQRKRHWKPSEDMQFVVMDATHPHHYFMDNVLGNPFPMDHISSTML

SEQ ID NO: 61 - protein, 257 aa, Arabidopsis thaliana
  peptide (1)..(257): ceres Seq. ID no. 13491863
MAHPHYHRLLAAYVNCQKVGAPPEVVARLEEACSSAAAAAASMGPTGCLGEDPGLDQFMEAYCEMLVKYEQELSKPFKEAMVFLQRVECQFKSLSLSSPSSFSGYGETAIDRNNNGSSEEEVDMNNEFVDPQAEDRELKGQLLRKYSGYLGSLKQEFMKKRKKGKLPKEARQQLLDWWSRHYKWPYPSEQQKLALAESTGLDQKQINNWFINQRKRHWKPSEDMQFVVMDATHPHHYFMDNVLGNPFPMDHISSTML

SEQ ID NO: 62 - DNA, 687 bp, Arabidopsis thaliana
  misc_feature (1)..(687): ceres Seq. ID no. 12325410
atcagaaaacaggaataaaagcactcaaaagtcaaagcgcgcgcgcgcacacacacacaaaaaacaatccaaagctaattgattcatcaatggcgatgtcgggaacatacgtgacggatgtgcctcttaagggatcggcggagaaacactataagaggtggaggagcgagaaccacctcgtccctgaagctatcggccacctcatccaaggtgtcaccgtccatgacggcgattgggactctcacggcaccatcaaaatctggaactacactcgtgatgggaaagaggaagtattgaaggagaggatagagatggacgacgagaatatggcggtgacgatcaatggactcgatggtcacgtcatggaggtcctcaaggtttatgtcacgacgttacatttcatccccgagtccaaagatggctgcgtctgcaaaattactatggtctgggagaaacgcaccgaagactccccagagcccatcgaattcatgaagttcgtggaacagatgattgctcacatggacgaccacatcctccaaaaccaggaatgatggatcgatgcttaatcatcatcacatcctccttttatttatcttaattaatgtgtgtccatctcgtacatgatattaatatcatcatggtgttgtatgtccaacgtttatttataaaagtattgttgttcgtttc

SEQ ID NO: 63 - protein, 182 aa, Arabidopsis thaliana
  peptide (1)..(182): ceres Seq. ID no. 12325411
QKTGIKALKSQSARAHTHTKNNPKLIDSSMAMSGTYVTDVPLKGSAEKHYKRWRSENHLVPEAIGHLIQGVTVHDGDWDSHGTIKIWNYTRDGKEEVLKERIEMDDENMAVTINGLDGHVMEVLKVYVTTLHFIPESKDGCVCKITMVWEKRTEDSPEPIEFMKFVEQMIAHMDDHILQNQE

SEQ ID NO: 64 - protein, 153 aa, Arabidopsis thaliana
  peptide (1)..(153): ceres Seq. ID no. 12325412
MAMSGTYVTDVPLKGSAEKHYKRWRSENHLVPEAIGHLIQGVTVHDGDWDSHGTIKIWNYTRDGKEEVLKERIEMDDENMAVTINGLDGHVMEVLKVYVTTLHFIPESKDGCVCKITMVWEKRTEDSPEPIEFMKFVEQMIAHMDDHILQNQE

SEQ ID NO: 65 - protein, 151 aa, Arabidopsis thaliana
  peptide (1)..(151): ceres Seq. ID no. 12325413
MSGTYVTDVPLKGSAEKHYKRWRSENHLVPEAIGHLIQGVTVHDGDWDSHGTIKIWNYTRDGKEEVLKERIEMDDENMAVTINGLDGHVMEVLKVYVTTLHFIPESKDGCVCKITMVWEKRTEDSPEPIEFMKFVEQMIAHMDDHILQNQE

SEQ ID NO: 66 - DNA, 602 bp, Arabidopsis thaliana
  misc_feature (1)..(602): ceres Seq. ID no. 12561621
agagtttcttccatctctcgctctttcgattttggagatatttacaggtacgcttgcttttcccttcacaacatcttcttcttcctgctcctctgtttcaaattccgaaaatcagataaaaaatgggagcttcgaaactttccgggatgcaaaagcaagtcttgagtctctacagaggatttctaagagcagctcgttcaagaccaatagaagacaggaagagaatagagatgattgtgtcaacagagttccgccacaactctaaggaggtcgaccgtaagaattttcagtacatcgagtatttgcttcggttaggtactaaacagcttgatcaactcaaaagccctgccatagttagcctctcctcgatcaaggtcgttacatctaaaacctgataccttattttatagcttgttgctctagtgaccatagataacaaagctgaggaaattgattcgttttcctctttttacattgagttatgtatgtatgatgatggatgttcaaggtccattataaaataaagagaattgtgatcacaattgaatatatatatacactttcacacaattatgtgcatctgctaatagtctagagattgc

SEQ ID NO: 67 - protein, 90 aa, Arabidopsis thaliana
  peptide (1)..(90): ceres Seq. ID no. 12561622
MGASKLSGMQKQVLSLYRGFLRAARSRPIEDRKRIEMIVSTEFRHNSKEVDRKNFQYIEYLLRLGTKQLDQLKSPAIVSLSSIKVVTSKT

SEQ ID NO: 68 - protein, 87 aa, Arabidopsis thaliana
  peptide (1)..(87): ceres Seq. ID no. 12561623
EFLPSLALSILEIFTGTLAFPFTTSSSSCSSVSNSENQIKNGSFETFRDAKASLESLQRISKSSSFKTNRRQEENRDDCVNRVPPQL

SEQ ID NO: 69 - protein, 82 aa, Arabidopsis thaliana
  peptide (1)..(82): ceres Seq. ID no. 12561624
MQKQVLSLYRGFLRAARSRPIEDRKRIEMIVSTEFRHNSKEVDRKNFQYIEYLLRLGTKQLDQLKSPAIVSLSSIKVVTSKT

SEQ ID NO: 70 - DNA, 763 bp, Arabidopsis thaliana
  misc_feature (1)..(763): ceres Seq. ID no. 12422873
aaaaaaacacaatccctcttagaaacagagagcttctcttctttctccattattcatcactttcttccccgaagatgcgtcctttagacgagaccgagacaaccgtcgtcttcgagaagctcttcaaattcgtcggcaacaacctcaagaagatcgtcgagaacccatcagacgaaggacccgaatctacacccggcagctactgcttccgcctccagaaaaacagagtctactacgtcagcgaagccctcgtgaagcgagccacaaacatctccaggaaaaacctagtctccttcggaacctgcatcgggaaatacactcacgccggtagtttccatctgacgattatgtcactcaatatcttggcggcgaatgctaaacacaaggtatggcttaaacccacttcggagatgtcgtttctttatgggaaccatgtcttgaaaggtggattagggaggattactgatagtattgttcctggagatggtgttgttgtgttctcgatgtctgatgttccgttagggtttgggattgctgcgaagagtacacaggattgtaggaaattggatcctaatggtattgttgtgcttcatcaggctgatattggagagtatttgaggggtgaagatgacctttgaaagtttaaaggttttgtctttgttgtgtcttagtttgattctgcttgttgctttgagttttaaagtatgtagctgttacaagtttcgtggaacttggttctggatctatgaaagtctcgattttt

SEQ ID NO: 71 - protein, 211 aa, Arabidopsis thaliana
  peptide (1)..(211): ceres Seq. ID no. 12422874
KNTIPLRNRELLFFLHYSSLSSPKMRPLDETETTVVFEKLFKFVGNNLKKIVENPSDEGPESTPGSYCFRLQKNRVYYVSEALVKRATNISRKNLVSFGTCIGKYTHAGSFHLTIMSLNILAANAKHKVWLKPTSEMSFLYGNHVLKGGLGRITDSIVPGDGVVVFSMSDVPLGFGIAAKSTQDCRKLDPNGIVVLHQADIGEYLRGEDDL

SEQ ID NO: 72 - protein, 187 aa, Arabidopsis thaliana
  peptide (1)..(187): ceres Seq. ID no. 12422875
MRPLDETETTVVFEKLFKFVGNNLKKIVENPSDEGPESTPGSYCFRLQKNRVYYVSEALVKRATNISRKNLVSFGTCIGKYTHAGSFHLTIMSLNILAANAKHKVWLKPTSEMSFLYGNHVLKGGLGRITDSIVPGDGVVVFSMSDVPLGFGIAAKSTQDCRKLDPNGIVVLHQADIGEYLRGEDDL

SEQ ID NO: 73 - DNA, 851 bp, Arabidopsis thaliana
  misc_feature (1)..(851): ceres Seq. ID no. 12393506
ataagatcttctttaaaatccaaaatcgtaggccactatttcattatacttatgtaatatatgtgatacagatacatctatatacaaattaaacacgaaaccatacatgcacggtgtgatcacacacgcacacacatagaaacataaacacgcaataatttctatacagtttaatttcatttttaacttacttctttttttttggtgaagattcttgagagaaaagaaatcgaagatggcaacaaaatccaccggaggtaccgagaaaaccaagtcgatagaagtgaagaagaaactaatcaacgtgttgatcgtcgatgatgatccattaaaccgtagactccacgagatgatcatcaaaacgatcggaggaatttctcagactgcaaagaatggcgaggaggcagtgatcctccaccgtgacggcgaagcatctttcgaccttattctaatggataaggaaatgcctgagagggatggagtttcgacaactaagaagctaagagaaatgaaagtgacgtcaatgatcgttggggtaacgtcattagctgaccaagaagaagagcgtaaggcttttatggaagctgggctcaaccattgcttggaaaaacccttaaccaaggccaagatcttcccgctcattagccacctcttcgatgcttgatggatgaaggctcattaatgtatctatattttcaatcatgaaatcacctacacgtgtatttgacacaaaaatctgcatttgttgtgatatagggtttctcatatctatgtttgatttattttcttatcgtccgaggtaaaatcatgcaagtcatttcttttggctaataaaatattaaaataagggc

SEQ ID NO: 74 - protein, 142 aa, Arabidopsis thaliana
  peptide (1)..(142): ceres Seq. ID no. 12393507
MATKSTGGTEKTKSIEVKKKLINVLIVDDDPLNRRLHEMIIKTIGGISQTAKNGEEAVILHRDGEASFDLILMDKEMPERDGVSTTKKLREMKVTSMIVGVTSLADQEEERKAFMEAGLNHCLEKPLTKAKIFPLISHLFDA

SEQ ID NO: 75 - protein, 104 aa, Arabidopsis thaliana
  peptide (1)..(104): ceres Seq. ID no. 12393508
MIIKTIGGISQTAKNGEEAVILHRDGEASFDLILMDKEMPERDGVSTTKKLREMKVTSMIVGVTSLADQEEERKAFMEAGLNHCLEKPLTKAKIFPLISHLFDA

SEQ ID NO: 76 - DNA, 834 bp, Arabidopsis thaliana
  misc_feature (1)..(834): ceres Seq. ID no. 12332848
ttccactttaaatcaggaacaaacccttcactgcgccagtgttgtgcggctctgcgaatcttcgaaaatgggtgcgtacaagtatgtatctgagctatggaggaagaaacagtccgatgtgatgagattcctccagagggttaggtgctgggagtacagacagcaaccttcgattgttcgtctcgtcaggcctactcgtcctgataaggctcgtcgtttgggttacaaggccaagcagggctttgttgtgtaccgtgtacgtgtgagacgtggtggacgcaagaggccagtgcctaagggtattgtgtatggtaagccaacaaaccagggagtgacacaactcaagttccagcgtagcaagcgttctgttgctgaggagcgtgctggcaggaaattgggtggtctcagagttgtcaactcttactggctcaatgaggattcgacctacaagtactacgagatcatcttggttgacccagcacacaatgctgtgcgtaatgacccgagaatcaactggatctgcaacccagttcacaagcaccgtgagctcagaggacttacctcagagggaaagaagaacagaggtctccgcggaaagggtcacaacaaccacaagaaccgtccatctcgcagggctacatggaagaaaaacaactctctcagccttcgtcgttaccgttgattgccttttgcttttattattactgttatcatcttctaggttccatgttgttgcacttttatctctctcaggatgacttgtttttgcaaattttggagactatacaattatgctttgaacctaaattttatatttgctttgcttttttgtcg

SEQ ID NO: 77 - protein, 226 aa, Arabidopsis thaliana
  peptide (1)..(226): ceres Seq. ID no. 12332849
STLNQEQTLHCASVVRLCESSKMGAYKYVSELWRKKQSDVMRFLQRVRCWEYRQQPSIVRLVRPTRPDKARRLGYKAKQGFVVYRVRVRRGGRKRPVPKGIVYGKPTNQGVTQLKFQRSKRSVAEERAGRKLGGLRVVNSYWLNEDSTYKYYEIILVDPAHNAVRNDPRINWICNPVHKHRELRGLTSEGKKNRGLRGKGHNNHKNRPSRRATWKKNNSLSLRRYR

SEQ ID NO: 78 - protein, 204 aa, Arabidopsis thaliana
  peptide (1)..(204): ceres Seq. ID no. 12332850
MGAYKYVSELWRKKQSDVMRFLQRVRCWEYRQQPSIVRLVRPTRPDKARRLGYKAKQGFVVYRVRVRRGGRKRPVPKGIVYGKPTNQGVTQLKFQRSKRSVAEERAGRKLGGLRVVNSYWLNEDSTYKYYEIILVDPAHNAVRNDPRINWICNPVHKHRELRGLTSEGKKNRGLRGKGHNNHKNRPSRRATWKKNNSLSLRRYR

SEQ ID NO: 79 - protein, 186 aa, Arabidopsis thaliana
  peptide (1)..(186): ceres Seq. ID no. 12332851
MRFLQRVRCWEYRQQPSIVRLVRPTRPDKARRLGYKAKQGFVVYRVRVRRGGRKRPVPKGIVYGKPTNQGVTQLKFQRSKRSVAEERAGRKLGGLRVVNSYWLNEDSTYKYYEIILVDPAHNAVRNDPRINWICNPVHKHRELRGLTSEGKKNRGLRGKGHNNHKNRPSRRATWKKNNSLSLRRYR

SEQ ID NO: 80 - DNA, 1794 bp, Arabidopsis thaliana
  misc_feature (1)..(1794): ceres Seq. ID no. 12558789
ctcagcagcttcttctgctttcaattactctcgccgacgattttctcaccggaaaaaaacaatatcattgcggatacacaaactataatggacctcctcttgctggagaagtctttaatcgccgtcttcgtggcggtgattctcgccacggtgatttcaaagctccgcggcaagaaattgaagctacctccaggtcctataccaattccgatcttcggaaactggcttcaagtcggagatgatctcaaccaccgtaatctcgtcgattacgctaagaaattcggcgatctcttcctcctccgtatgggtcagcgaaacctagtcgtcgtctcctcaccggatctaacaaaggaagtgctcctcactcaaggcgttgagtttggatccagaacgagaaacgtcgtgttcgacattttcaccgggaaaggtcaagatatggtgttcactgtttacggcgagcattggaggaagatgagaagaatcatgacggttcctttcttcaccaacaaagttgttcaacagaatcgtgaaggttgggagtttgaagcagctagtgttgttgaagatgttaagaagaatccagattctgctacgaaaggaatcgtgttgaggaaacgtttgcaattgatgatgtataacaatatgttccgtatcatgttcgatagaagatttgagagtgaggatgatcctcttttccttaggcttaaggctttgaatggtgagagaagtcgattagctcagagctttgagtataactatggagatttcattcctatccttagaccattcctcagaggctatttgaagatttgtcaagatgtgaaagatcgaagaatcgctcttttcaagaagtactttgttgatgagaggaagcaaattgcgagttctaagcctacaggtagtgaaggattgaaatgtgccattgatcacatccttgaagctgagcagaagggagaaatcaacgaggacaatgttctttacatcgtcgagaacatcaatgtcgccgcgattgagacaacattgtggtctatcgagtggggaattgcagagctagtgaaccatcctgaaatccagagtaagctaaggaacgaactcgacacagttcttggaccgggtgtgcaagtcaccgagcctgatcttcacaaacttccataccttcaagctgtggttaaggagactcttcgtctgagaatggcgattcctctcctcgtgcctcacatgaacctccatgatgcgaagctcgctggctacgatatcccagcagaaagcaaaatccttgttaatgcttggtggctagcaaacaaccccaacagctggaagaagcctgaagagtttagaccagagaggttctttgaagaagaatcgcacgtggaagctaacggtaatgacttcaggtatgtgccatttggtgttggacgtcgaagctgtcccgggattatattggcattgcctattttggggatcaccattggtaggatggtccagaacttcgagcttcttcctcctccaggacagtctaaagtggatactagtgagaaaggtggacaattcagcttgcacatccttaaccactccataatcgttatgaaaccaaggaactgttaaactttctgcacaaaaaaaaggatgaagatgactttataaatgtttgtgaaatctgttgaaatattcccttgttttgcttttgtgagatgtttttgtgtaaaatgtctttaaatggttcgttctacgattgcaataataattagtggtgctcattgttttggatggatcaatgttatacttatatcattt

SEQ ID NO: 81 - protein, 534 aa, Arabidopsis thaliana
  peptide (1)..(534): ceres Seq. ID no. 12558790
LSSFFCFQLLSPTIFSPEKNNIIADTQTIMDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 82 - protein, 505 aa, Arabidopsis thaliana
  peptide (1)..(505): ceres Seq. ID no. 12558791
MDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 83 - protein, 433 aa, Arabidopsis thaliana
  peptide (1)..(433): ceres Seq. ID no. 12558792
MGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 84 - DNA, 1057 bp, Arabidopsis thaliana
  misc_feature (1)..(1057): ceres Seq. ID no. 12738854
gtttttgggattctcaaaaaactcaaaataaactttcctcattttttttttctttcttttgttttgttttctttataaaggagaaagaaagagaaagagagtttccgaaagctgaattgagttgggtgaagcaaattctcaggatgggaagaggaaagatagaaataaagaggatagagaactcaacaaatcgacaagtgacgttttgcaaaagaagaaatggacttctgaagaaagcctatgagctttcggtcctttgcgatgcagaagttgcgctcattgttttctccactcgtggccgtctctatgaatacgccaataacaacataagatcaaccattgagaggtacaagaaagcttgttctgatagcaccaacactagcactgtccaagaaatcaatgccgcgtactatcaacaagaatctgctaagctgagacaacagatccaaacgattcaaaactccaacaggaatctgatgggagactctttgagttccttaagtgtcaaggaactaaaacaagttgagaatcgccttgagaaagctatctctaggatcaggtccaagaagcatgagttgcttctagttgaaatcgaaaacgcgcagaaaagggagattgagcttgacaatgagaacatctatctaagaactaaggtagcagaagtggagaggtatcaacaacaccatcatcaaatggttagtggttcagagattaacgcaattgaagctttagcctcacgcaattactttgctcatagcattatgactgctggttctggatctggtaatggaggttcttactctgatcccgacaagaaaattcttcatctcggataatctcgtctgcgaaaaaccgagctgcaataactctctgcatgcatctggggattctttgctcagatttatctctacataaaatatgttttggtgcaaaataattcaaagtacaatgaaggtattggtccttcaagactcattgcaatttgtgttgtgtaatgattaagacatactctagtttttattgcttcgtatcatagtatctattctatggtatctcc

SEQ ID NO: 85 - protein, 277 aa, Arabidopsis thaliana
  peptide (1)..(277): ceres Seq. ID no. 12738855
FWDSQKTQNKLSSFFFSFFCFVFFIKEKEREREFPKAELSWVKQILRMGRGKIEIKRIENSTNRQVTFCKRRNGLLKKAYELSVLCDAEVALIVFSTRGRLYEYANNNIRSTIERYKKACSDSTNTSTVQEINAAYYQQESAKLRQQIQTIQNSNRNLMGDSLSSLSVKELKQVENRLEKAISRIRSKKHELLLVEIENAQKREIELDNENIYLRTKVAEVERYQQHHHQMVSGSEINAIEALASRNYFAHSIMTAGSGSGNGGSYSDPDKKILHLG

SEQ ID NO: 86 - protein, 230 aa, Arabidopsis thaliana
  peptide (1)..(230): ceres Seq. ID no. 12738856
MGRGKIEIKRIENSTNRQVTFCKRRNGLLKKAYELSVLCDAEVALIVFSTRGRLYEYANNNIRSTIERYKKACSDSTNTSTVQEINAAYYQQESAKLRQQIQTIQNSNRNLMGDSLSSLSVKELKQVENRLEKAISRIRSKKHELLLVEIENAQKREIELDNENIYLRTKVAEVERYQQHHHQMVSGSEINAIEALASRNYFAHSIMTAGSGSGNGGSYSDPDKKILHLG

SEQ ID NO: 87 - DNA, 1623 bp, Arabidopsis thaliana
  misc_feature (1)..(1623): ceres Seq. ID no. 13487143
ccgagattacagagaaccccgtcccgtgtttaaaccctccggcgacaaagctcagatagagaaccgctccacccttgtcgttctctgcagccatgtacggcggagatgaagtgtcagctattgtggttgacttgggttcgcacacttgcaaggctggttacgccggtgaagatgctcccaaggccgttttcccttccgttattggggcagtagatggtgtagaggcaatggatgtggatgttgattctacaaagactaattcgaactctgaggattcgaagaccgagtctgaaaaggaaaagggcaaacgcaagctctatgtaggatctcaagccatgagctaccgcagagatcatatggaggttttgtcacctataaaagatggcatcgtttctgactgggatttggtggacaatatatgggaacatgctttcaagagttgtctgatgattgatcctacggagcatccaatgctgctagctgagcctcctttaaacactcaacagcagagagagaaggcggcagagctaatgtttgaaaagtacaaagttccggcactgtttatggctaagaaccctgttctcacgtcttttgctactgggcgtgctacttctttggttgttgactgcggtggaggatccacaaccatttcaccggtgcatgatggttatgttcttcagaaggctgttgtatcgtctccacttggaggggaatttctcactgactgcctactgaaaagcttggagagtaaaggaattaaaataagacctagatactctttcaagaggaaggaagtacgagcaggggaattccaggttgaagatgtggatattcctgacaccacggaaagctacaaactcttctgccagaggatgatagtgggtgatatcaaggattctatttgtcgagttcctgatactccctatgacgataaatcatattcaaacatccctactacgtcgtacgagcttcctgatggccagacgctcgagattggtgctgataggttcaaagttcctgatgtgatgttcaacccgtccatagtccagacgattcctggaatggagaagtatgctgatatgattccttccgttcgtgggttaccacacatggtcatggagagcatcaacaaatgtgatgtagacatccggagagagttgtatagtagcatactgcttgctggtggcacatcgtcaatgcaacagctgaaagaacgtctcgaaaaggacttgatcgaagagtctcctcactcagctagagttaaagtgttggcaagcggtaacacaacagaaagaagattcagtgtatggataggagggagtatattggcatcactaggctcatttcaacagatgtggttctccaagtctgagtatgaggaacatggagcttcctacattcagagaaaatgcccttaaagcaggcagtttcagattatgtggagtctgacaagacgaaacttggaaactaatgtgttattttcattgtgcatcgttgttccttgaagattattagtattccgcctgaaccttttagaatgagtgtcattaacaagcattatagtgctatttttaaaacataaaaacatgtggaagaagtttcgttgctttattagattttcgc

SEQ ID NO: 88 - protein, 441 aa, Arabidopsis thaliana
  peptide (1)..(441): ceres Seq. ID no. 13487144
MYGGDEVSAIVVDLGSHTCKAGYAGEDAPKAVFPSVIGAVDGVEAMDVDVDSTKTNSNSEDSKTESEKEKGKRKLYVGSQAMSYRRDHMEVLSPIKDGIVSDWDLVDNIWEHAFKSCLMIDPTEHPMLLAEPPLNTQQQREKAAELMFEKYKVPALFMAKNPVLTSFATGRATSLVVDCGGGSTTISPVHDGYVLQKAVVSSPLGGEFLTDCLLKSLESKGIKIRPRYSFKRKEVRAGEFQVEDVDIPDTTESYKLFCQRMIVGDIKDSICRVPDTPYDDKSYSNIPTTSYELPDGQTLEIGADRFKVPDVMFNPSIVQTIPGMEKYADMIPSVRGLPHMVMESINKCDVDIRRELYSSILLAGGTSSMQQLKERLEKDLIEESPHSARVKVLASGNTTERRFSVWIGGSILASLGSFQQMWFSKSEYEEHGASYIQRKCP

SEQ ID NO: 89 - protein, 396 aa, Arabidopsis thaliana
  peptide (1)..(396): ceres Seq. ID no. 13487145
MDVDVDSTKTNSNSEDSKTESEKEKGKRKLYVGSQAMSYRRDHMEVLSPIKDGIVSDWDLVDNIWEHAFKSCLMIDPTEHPMLLAEPPLNTQQQREKAAELMFEKYKVPALFMAKNPVLTSFATGRATSLVVDCGGGSTTISPVHDGYVLQKAVVSSPLGGEFLTDCLLKSLESKGIKIRPRYSFKRKEVRAGEFQVEDVDIPDTTESYKLFCQRMIVGDIKDSICRVPDTPYDDKSYSNIPTTSYELPDGQTLEIGADRFKVPDVMFNPSIVQTIPGMEKYADMIPSVRGLPHMVMESINKCDVDIRRELYSSILLAGGTSSMQQLKERLEKDLIEESPHSARVKVLASGNTTERRFSVWIGGSILASLGSFQQMWFSKSEYEEHGASYIQRKCP

SEQ ID NO: 90 - protein, 360 aa, Arabidopsis thaliana
  peptide (1)..(360): ceres Seq. ID no. 13487146
MSYRRDHMEVLSPIKDGIVSDWDLVDNIWEHAFKSCLMIDPTEHPMLLAEPPLNTQQQREKAAELMFEKYKVPALFMAKNPVLTSFATGRATSLVVDCGGGSTTISPVHDGYVLQKAVVSSPLGGEFLTDCLLKSLESKGIKIRPRYSFKRKEVRAGEFQVEDVDIPDTTESYKLFCQRMIVGDIKDSICRVPDTPYDDKSYSNIPTTSYELPDGQTLEIGADRFKVPDVMFNPSIVQTIPGMEKYADMIPSVRGLPHMVMESINKCDVDIRRELYSSILLAGGTSSMQQLKERLEKDLIEESPHSARVKVLASGNTTERRFSVWIGGSILASLGSFQQMWFSKSEYEEHGASYIQRKCP

SEQ ID NO: 91 - DNA, 523 bp, Arabidopsis thaliana
  misc_feature (1)..(523): ceres Seq. ID no. 12337118
atcaaggtaagacacttcgcttcaaatcatttgtgtattacagaaacttctgatcgtcatggaagctacttcacaaccatgtttctctcataactcctcacttcattcgacccgtaagcaaccggcaaacccatggaaaaaacccgtaacgggcttgcctcagaggatgcatcctaaagtttacagagtcgagcctgttaacttcaaggagttagttcagaggctaactggtgcagaagacgttgagcaagaggtacatcaagttgaagctaaaccagtcaagatttctgatgatacaacaacagctaaggataatccgttcgcgtttgatctttcaccttcgtcatctcggttctgggaagctttccctctgcttagtcctgctaatctatcaagatggtagcgagaaagaagctattcttgccaatttttcggcaattcatgaatttgtttcatcagaattagaactgtagtagtaacactttatgatcttatctttaaatacaaaagttactgttacatt

SEQ ID NO: 92 - protein, 114 aa, Arabidopsis thaliana
  peptide (1)..(114): ceres Seq. ID no. 12337119
MEATSQPCFSHNSSLHSTRKQPANPWKKPVTGLPQRMHPKVYRVEPVNFKELVQRLTGAEDVEQEVHQVEAKPVKISDDTTTAKDNPFAFDLSPSSSRFWEAFPLLSPANLSRW

SEQ ID NO: 93 - protein, 78 aa, Arabidopsis thaliana
  peptide (1)..(78): ceres Seq. ID no. 12337120
MHPKVYRVEPVNFKELVQRLTGAEDVEQEVHQVEAKPVKISDDTTTAKDNPFAFDLSPSSSRFWEAFPLLSPANLSRW

SEQ ID NO: 94 - protein, 60 aa, Arabidopsis thaliana
  peptide (1)..(60): ceres Seq. ID no. 12337121
MIQQQLRIIRSRLIFHLRHLGSGKLSLCLVLLIYQDGSEKEAILANFSAIHEFVSSELEL

SEQ ID NO: 95 - DNA, 1081 bp, Arabidopsis thaliana
  misc_feature (1)..(1081): ceres Seq. ID no. 12333219
attatcatatcctcttcttccttcttcactatcaatcttcttctccactacaacacaaatggagaaatcatcctcaatgaaacaatggaagaagggtcctgctcggggtaaaggcggtccacaaaacgctctttgtcagtaccgtggagtcaggcaaaggacttggggcaaatgggtggctgagatcagagagcccaagaagagggcaagactttggcttggctctttcgctacagctgaagaagcagctatggcttatgatgaggctgccttgaaactctatgggcacgacgcatacctcaacttacctcatcttcagcggaatacaagaccttctctgagtaactctcagaggttcaaatgggtaccttcaaggaagtttatatctatgtttccttcatgtggtatgctaaacgtgaatgctcagcctagtgttcacataatccagcaaagactagaagaactcaagaaaactggacttttatctcaatcctattcttctagttcttcctccaccgaatcaaaaactaatactagctttcttgatgagaagaccagcaagggagaaacagacaatatgttcgaaggtggtgatcagaagaaaccagagatcgacctgaccgagtttcttcagcaactaggaatcttgaaggatgaaaatgaagcagaaccaagtgaggtagcagagtgtcattcccctccaccatggaacgagcaagaagaaactggaagtcctttcagaactgagaatttcagctgggataccctgatcgagatgccaagaagtgaaaccacaactatgcaatttgactccagcaacttcggaagctatgattttgaggatgatgtatccttcccttccatctgggactactacggaagcttagattgagtaaaagcaatttaaggtagatcaagattcagaagtacacaaatggttttggatttagtgtagcgttttggaaaagagacataggtagtgagagtgcagtcttttattatgcagcaataaagtgagtcagtgtacaaccgagttgttcgctttttttggtatattaattaagcatgttcattttttcgc

SEQ ID NO: 96 - protein, 296 aa, Arabidopsis thaliana
  peptide (1)..(296): ceres Seq. ID no. 12333220
LSYPLLPSSLSIFFSTTTQMEKSSSMKQWKKGPARGKGGPQNALCQYRGVRQRTWGKWVAEIREPKKRARLWLGSFATAEEAAMAYDEAALKLYGHDAYLNLPHLQRNTRPSLSNSQRFKWVPSRKFISMFPSCGMLNVNAQPSVHIIQQRLEELKKTGLLSQSYSSSSSSTESKTNTSFLDEKTSKGETDNMFEGGDQKKPEIDLTEFLQQLGILKDENEAEPSEVAECHSPPPWNEQEETGSPFRTENFSWDTLIEMPRSETTTMQFDSSNFGSYDFEDDVSFPSIWDYYGSLD

SEQ ID NO: 97 - protein, 277 aa, Arabidopsis thaliana
  peptide (1)..(277): ceres Seq. ID no. 12333221
MEKSSSMKQWKKGPARGKGGPQNALCQYRGVRQRTWGKWVAEIREPKKRARLWLGSFATAEEAAMAYDEAALKLYGHDAYLNLPHLQRNTRPSLSNSQRFKWVPSRKFISMFPSCGMLNVNAQPSVHIIQQRLEELKKTGLLSQSYSSSSSSTESKTNTSFLDEKTSKGETDNMFEGGDQKKPEIDLTEFLQQLGILKDENEAEPSEVAECHSPPPWNEQEETGSPFRTENFSWDTLIEMPRSETTTMQFDSSNFGSYDFEDDVSFPSIWDYYGSLD

SEQ ID NO: 98 - protein, 271 aa, Arabidopsis thaliana
  peptide (1)..(271): ceres Seq. ID no. 12333222
MKQWKKGPARGKGGPQNALCQYRGVRQRTWGKWVAEIREPKKRARLWLGSFATAEEAAMAYDEAALKLYGHDAYLNLPHLQRNTRPSLSNSQRFKWVPSRKFISMFPSCGMLNVNAQPSVHIIQQRLEELKKTGLLSQSYSSSSSSTESKTNTSFLDEKTSKGETDNMFEGGDQKKPEIDLTEFLQQLGILKDENEAEPSEVAECHSPPPWNEQEETGSPFRTENFSWDTLIEMPRSETTTMQFDSSNFGSYDFEDDVSFPSIWDYYGSLD

SEQ ID NO: 99 - DNA, 3591 bp, Arabidopsis thaliana
  misc_feature (1)..(3591): ceres Seq. ID no. 12653917
atgaagactttttcaagcttctttctctctgtaacaactctcttcttcttctccttcttttctctttcatttcaagcttcaccatctcagtctttatacagagaaatccatcagcttataagcttcaaagacgttcttcctgacaagaatcttctcccagactggtcttccaacaaaaacccgtgtactttcgatggcgttacttgcagagacgacaaagttacttcgattgatctcagctccaagcctctcaacgtcggattcagtgccgtgtcctcgtctctcctgtctctcaccggattagagtctctgtttctctcaaactcacacatcaatggctccgtttctggcttcaagtgctctgcttctttaaccagcttggatctatctagaaactctctttcgggtcctgtaacgactctaacaagccttggttcttgctccggtctgaagtttcttaacgtctcttccaatacacttgattttcccgggaaagtttcaggtgggttgaagctaaacagcttggaagttctggatctttctgcgaattcaatctccggtgctaacgtcgttggttgggttctctccgatgggtgtggagagttgaaacatttagcgattagcggaaacaaaatcagtggagacgtcgatgtttctcgctgcgtgaatctcgagtttctcgatgtttcctccaacaatttctccactgggattcctttcctcggagattgctctgctctgcaacatcttgacatctccgggaacaaattatccggcgatttctcccgtgctatctctacttgcacagagctcaagttgttgaacatctctagtaaccaattcgtcggaccaatccctccgctaccgcttaaaagtctccaatacctctctctggccgagaacaaattcaccggcgagatccctgactttctctccggcgcgtgtgatacactcactggtctcgatctctctggaaatcatttctacggtgcggttcctccattcttcggttcatgttctcttctcgaatcactcgcgttgtcgagtaacaacttctctggcgagttaccgatggatacgttgttgaagatgagaggactcaaagtacttgatctgtctttcaacgagttttccggcgaattaccggaatctctgacgaatctatccgcttcgttgctaacgttagatctcagctccaacaatttctccggtccgattctcccaaatctctgccagaaccctaaaaacactctgcaggagctttaccttcagaacaatggcttcaccgggaagattccaccgactttaagcaactgttctgagctggtttcgcttcacttgagcttcaattacctctccgggacaatcccttcgagcttaggctctctatcgaagcttcgagatctgaaactatggctgaatatgttagaaggagagatccctcaggagctcatgtatgtcaagaccttagagactctgatcctcgacttcaacgatttaaccggtgaaatcccttccggtttaagtaactgtaccaatcttaactggatttctctgtcgaataaccggttaaccggtgagattccgaaatggattggccggttagagaatctcgctatcctcaagctaagcaacaattcattctccgggaacattccggctgagctcggcgactgcagaagcttaatctggcttgatctcaacaccaatctcttcaatggaacgattccggcggcgatgtttaaacaatccgggaaaatcgctgccaatttcatcgccggtaagaggtacgtttatatcaaaaacgatgggatgaagaaagagtgtcatggagctggtaatttacttgagtttcaaggaatcagatccgaacaattaaaccggctttcaacgaggaacccttgtaatatcactagcagagtctatggaggtcacacttcgccgacgtttgataacaatggttcgatgatgtttctggacatgtcttacaacatgttgtctggatacataccgaaggagattggttcgatgccttatctgtttattctcaatttgggtcataacgatatctctggttcgattcctgatgaggtaggtgatctaagaggtttaaacattcttgatctttcaagcaataagctcgatgggaggattcctcaggctatgtcagctcttactatgcttacggaaatcgatttgtcgaataataatttgtctggtccgattcctgagatgggtcagtttgagacttttccaccggctaagttcttgaacaatcctggtctctgtggttatcctcttccgcggtgtgatccttcaaatgcagacggttatgctcatcatcagagatctcatggaaggagaccagcgtcccttgctggtagtgtggcgatgggattgttgttctcttttgtgtgtatatttgggctgatccttgttggtagagagatgaggaagagacggagaaagaaagaggcggagttggagatgtatgcggaaggacatggaaactctggcgatagaactgctaacaacaccaattggaagctgactggtgtgaaagaagccttgagtatcaatcttgctgctttcgagaagccattgcggaagctcacgtttgcggatcttcttcaggctaccaatggtttccataatgatagtctgattggttctggtgggtttggagatgtttacaaagcgattttgaaagatggaagcgcggtggctatcaagaaactgattcatgttagcggtcaaggtgatagagagttcatggcggagatggaaaccattgggaagatcaaacatcgaaatcttgtgcctcttcttggttattgcaaagttggagacgagcggcttcttgtgtatgagtttatgaagtatggaagtttagaagatgttttgcacgaccccaagaaagctggggtgaaactaaactggtccacacggcggaagattgcgataggatcagctagagggcttgctttccttcaccacaactgcagtccgcatatcatccacagagacatgaaatccagtaatgtgttgcttgatgagaatttggaagctcgggtttcagattttggcatggcgaggctgatgagtgcgatggatacgcatttaagcgtcagtacattagctggtacaccgggttacgttcctccagagtattaccaaagtttcaggtgttcaacaaaaggagacgtttatagttacggtgtggtcttactcgagctactcacgggtaaacggccaacggattcaccggattttggagataacaaccttgttggatgggtgaaacagcacgcaaaactgcggattagcgatgtgtttgaccccgagcttatgaaggaagatccagcattagagatcgaacttttacaacatttaaaagttgcggttgcgtgtttggatgatcgggcttggagacgaccgacaatggtacaagtcatggccatgtttaaggagatacaagccgggtcagggatagattcacagtcaacgatcagatcaatagaggatggagggttcagtacaatagagatggttgatatgagtataaaagaagttcctgaaggaaaattatga

SEQ ID NO: 100 - protein, 1196 aa, Arabidopsis thaliana
  peptide (1)..(1196): ceres Seq. ID no. 12653918
MKTFSSFFLSVTTLFFFSFFSLSFQASPSQSLYREIHQLISFKDVLPDKNLLPDWSSNKNPCTFDGVTCRDDKVTSIDLSSKPLNVGFSAVSSSLLSLTGLESLFLSNSHINGSVSGFKCSASLTSLDLSRNSLSGPVTTLTSLGSCSGLKFLNVSSNTLDFPGKVSGGLKLNSLEVLDLSANSISGANVVGWVLSDGCGELKHLAISGNKISGDVDVSRCVNLEFLDVSSNNFSTGIPFLGDCSALQHLDISGNKLSGDFSRAISTCTELKLLNISSNQFVGPIPPLPLKSLQYLSLAENKFTGEIPDFLSGACDTLTGLDLSGNHFYGAVPPFFGSCSLLESLALSSNNFSGELPMDTLLKMRGLKVLDLSFNEFSGELPESLTNLSASLLTLDLSSNNFSGPILPNLCQNPKNTLQELYLQNNGFTGKIPPTLSNCSELVSLHLSFNYLSGTIPSSLGSLSKLRDLKLWLNMLEGEIPQELMYVKTLETLILDFNDLTGEIPSGLSNCTNLNWISLSNNRLTGEIPKWIGRLENLAILKLSNNSFSGNIPAELGDCRSLIWLDLNTNLFNGTIPAAMFKQSGKIAANFIAGKRYVYIKNDGMKKECHGAGNLLEFQGIRSEQLNRLSTRNPCNITSRVYGGHTSPTFDNNGSMMFLDMSYNMLSGYIPKEIGSMPYLFILNLGHNDISGSIPDEVGDLRGLNILDLSSNKLDGRIPQAMSALTMLTEIDLSNNNLSGPIPEMGQFETFPPAKFLNNPGLCGYPLPRCDPSNADGYAHHQRSHGRRPASLAGSVAMGLLFSFVCIFGLILVGREMRKRRRKKEAELEMYAEGHGNSGDRTANNTNWKLTGVKEALSINLAAFEKPLRKLTFADLLQATNGFHNDSLIGSGGFGDVYKAILKDGSAVAIKKLIHVSGQGDREFMAEMETIGKIKHRNLVPLLGYCKVGDERLLVYEFMKYGSLEDVLHDPKKAGVKLNWSTRRKIAIGSARGLAFLHHNCSPHIIHRDMKSSNVLLDENLEARVSDFGMARLMSAMDTHLSVSTLAGTPGYVPPEYYQSFRCSTKGDVYSYGVVLLELLTGKRPTDSPDFGDNNLVGWVKQHAKLRISDVFDPELMKEDPALEIELLQHLKVAVACLDDRAWRRPTMVQVMAMFKEIQAGSGIDSQSTIRSIEDGGFSTIEMVDMSIKEVPEGKL

SEQ ID NO: 101 - protein, 839 aa, Arabidopsis thaliana
  peptide (1)..(839): ceres Seq. ID no. 12653919
MDTLLKMRGLKVLDLSFNEFSGELPESLTNLSASLLTLDLSSNNFSGPILPNLCQNPKNTLQELYLQNNGFTGKIPPTLSNCSELVSLHLSFNYLSGTIPSSLGSLSKLRDLKLWLNMLEGEIPQELMYVKTLETLILDFNDLTGEIPSGLSNCTNLNWISLSNNRLTGEIPKWIGRLENLAILKLSNNSFSGNIPAELGDCRSLIWLDLNTNLFNGTIPAAMFKQSGKIAANFIAGKRYVYIKNDGMKKECHGAGNLLEFQGIRSEQLNRLSTRNPCNITSRVYGGHTSPTFDNNGSMMFLDMSYNMLSGYIPKEIGSMPYLFILNLGHNDISGSIPDEVGDLRGLNILDLSSNKLDGRIPQAMSALTMLTEIDLSNNNLSGPIPEMGQFETFPPAKFLNNPGLCGYPLPRCDPSNADGYAHHQRSHGRRPASLAGSVAMGLLFSFVCIFGLILVGREMRKRRRKKEAELEMYAEGHGNSGDRTANNTNWKLTGVKEALSINLAAFEKPLRKLTFADLLQATNGFHNDSLIGSGGFGDVYKAILKDGSAVAIKKLIHVSGQGDREFMAEMETIGKIKHRNLVPLLGYCKVGDERLLVYEFMKYGSLEDVLHDPKKAGVKLNWSTRRKIAIGSARGLAFLHHNCSPHIIHRDMKSSNVLLDENLEARVSDFGMARLMSAMDTHLSVSTLAGTPGYVPPEYYQSFRCSTKGDVYSYGVVLLELLTGKRPTDSPDFGDNNLVGWVKQHAKLRISDVFDPELMKEDPALEIELLQHLKVAVACLDDRAWRRPTMVQVMAMFKEIQAGSGIDSQSTIRSIEDGGFSTIEMVDMSIKEVPEGKL

SEQ ID NO: 102 - protein, 833 aa, Arabidopsis thaliana
  peptide (1)..(833): ceres Seq. ID no. 12653920
MRGLKVLDLSFNEFSGELPESLTNLSASLLTLDLSSNNFSGPILPNLCQNPKNTLQELYLQNNGFTGKIPPTLSNCSELVSLHLSFNYLSGTIPSSLGSLSKLRDLKLWLNMLEGEIPQELMYVKTLETLILDFNDLTGEIPSGLSNCTNLNWISLSNNRLTGEIPKWIGRLENLAILKLSNNSFSGNIPAELGDCRSLIWLDLNTNLFNGTIPAAMFKQSGKIAANFIAGKRYVYIKNDGMKKECHGAGNLLEFQGIRSEQLNRLSTRNPCNITSRVYGGHTSPTFDNNGSMMFLDMSYNMLSGYIPKEIGSMPYLFILNLGHNDISGSIPDEVGDLRGLNILDLSSNKLDGRIPQAMSALTMLTEIDLSNNNLSGPIPEMGQFETFPPAKFLNNPGLCGYPLPRCDPSNADGYAHHQRSHGRRPASLAGSVAMGLLFSFVCIFGLILVGREMRKRRRKKEAELEMYAEGHGNSGDRTANNTNWKLTGVKEALSINLAAFEKPLRKLTFADLLQATNGFHNDSLIGSGGFGDVYKAILKDGSAVAIKKLIHVSGQGDREFMAEMETIGKIKHRNLVPLLGYCKVGDERLLVYEFMKYGSLEDVLHDPKKAGVKLNWSTRRKIAIGSARGLAFLHHNCSPHIIHRDMKSSNVLLDENLEARVSDFGMARLMSAMDTHLSVSTLAGTPGYVPPEYYQSFRCSTKGDVYSYGVVLLELLTGKRPTDSPDFGDNNLVGWVKQHAKLRISDVFDPELMKEDPALEIELLQHLKVAVACLDDRAWRRPTMVQVMAMFKEIQAGSGIDSQSTIRSIEDGGFSTIEMVDMSIKEVPEGKL

SEQ ID NO: 103 - DNA, 1267 bp, glycine max
  misc_feature (1)..(1267): ceres Seq. ID no. 12431631
ggagtacacagtcaataacggtcacatgcgctttgctacttctctaaacgtgaaaacatttacaccaacctacactccttgctcctacatatcccgcgccttacatgcagttccaagtatcaccaaaccaagcagtgtgatactcatactcagtactcacagcaccactttcaagtgttcaaagttcaactatcccaatatcaacgaaccatggatacttgcaagaagtcccctttgaagccatggaagaaagggccaacaagggggaaaggcgggccccagaacgcctcctgcgagtaccgaggcgttcggcagcgaacttggggcaaatgggttgctgagataagagagccaaagaagagaaccaggctctggcttggttcttttgccacagctgaagaagctgccatggcttatgatgaggctgctaggagactctatggtccagatgcataccttaatctcccccacctgcaacccaggtccacttcaactatcacgtcaggaaagttcaaatggttcccttccaagaacttcatttcaatgtttccttcctgtggattactcaatgtaaatgctcaacctagtgttcatttaatccatcagaggctgcaagagcttaagcgaaattcagttgtgagtcaatcctcatctagttcatccaatgatccaaaggcagaaattcagaatgtagacagcaaaaatcacggagaagatgaaaatcctccaaaagatgttcaaacatcatcagaggaggtgcttggagatcttcaggagaaaccccagatagacctccatgagtttcttcaacagatgggaatacttaaagaagaaagacagtcagaaagaactgatagctcaggaagttcaacagtgcgtgaagctgtgttgacagatgactgtgatcatttgggagtgttttctgacaagagtgttaactgggaggcattgattgagatgcatggacttgcaggtattcaggaatcagaagtcactcagcttgaagcatatgagccaaatgatgatctaactttctcaacttccatttggaacttttaagaaacttagcatataagcctaagcaatagatcacaaggttttggttatccaactactctagtcctgtatatgtctgcagcatttcaatctgtcggcagcactgtaagagtaagaaagtagctagaatagaagactactagtgagaaaggagcttatttcagtagttgtgacagcaaatggagaaaataatatcagtctgcgtattatt

SEQ ID NO: 104 - protein, 282 aa, glycine max
  peptide (1)..(282): ceres Seq. ID no. 12431632
MDTCKKSPLKPWKKGPTRGKGGPQNASCEYRGVRQRTWGKWVAEIREPKKRTRLWLGSFATAEEAAMAYDEAARRLYGPDAYLNLPHLQPRSTSTITSGKFKWFPSKNFISMFPSCGLLNVNAQPSVHLIHQRLQELKRNSVVSQSSSSSSNDPKAEIQNVDSKNHGEDENPPKDVQTSSEEVLGDLQEKPQIDLHEFLQQMGILKEERQSERTDSSGSSTVREAVLTDDCDHLGVFSDKSVNWEALIEMHGLAGIQESEVTQLEAYEPNDDLTFSTSIWNF

SEQ ID NO: 105 - protein, 216 aa, glycine max
  peptide (1)..(216): ceres Seq. ID no. 12431633
MAYDEAARRLYGPDAYLNLPHLQPRSTSTITSGKFKWFPSKNFISMFPSCGLLNVNAQPSVHLIHQRLQELKRNSVVSQSSSSSSNDPKAEIQNVDSKNHGEDENPPKDVQTSSEEVLGDLQEKPQIDLHEFLQQMGILKEERQSERTDSSGSSTVREAVLTDDCDHLGVFSDKSVNWEALIEMHGLAGIQESEVTQLEAYEPNDDLTFSTSIWNF

SEQ ID NO: 106 - protein, 171 aa, glycine max
  peptide (1)..(171): ceres Seq. ID no. 12431634
MFPSCGLLNVNAQPSVHLIHQRLQELKRNSVVSQSSSSSSNDPKAEIQNVDSKNHGEDENPPKDVQTSSEEVLGDLQEKPQIDLHEFLQQMGILKEERQSERTDSSGSSTVREAVLTDDCDHLGVFSDKSVNWEALIEMHGLAGIQESEVTQLEAYEPNDDLTFSTSIWNF

SEQ ID NO: 107 - DNA, 728 bp, glycine max
  misc_feature (1)..(728): ceres Seq. ID no. 12447176
acatgcaacttgtcttaatttctttctcgatccccaacatcactagctagctccttttgtacacactctacaaccccacctagctacatcacttaattagttttcccatatctataaccaatttcaaattctcacccttaactagctagctatatttcataactgattattaccaactcactacatattattggctaggattcaccattagacttaaaagtagttgatttattatatatataagatgtctagcaggaggtcacggtcaaggcaaacaagtagttcaaggaatatcaccgatgatcagatcaatgatcttgtctccaagttgcaacagcttcttccagagattcgcgataggcgctctgacaaggtttcagcttccaaggtgttgcaagagacatgcaactatattagaagcttacacagggaagtggatgacctaagcgagcgtttatctgagctcttggctacaactgacacagcacaagctgcaataattagaaatctactaatgcaatagatcggtgcagtagttaatttatcgcataattcatagttagcacttcagtacttgtgaaccgatccagtcagtagtcgcgtatttcttattctctttttgtttcactttttttttctggtttttgtccactaatatgcatgattactgcttttgcaaagcccattttcctaagatattaaataaaagtctgagtttgcgctttgct

SEQ ID NO: 108 - protein, 92 aa, glycine max
  peptide (1)..(92): ceres Seq. ID no. 12447177
MSSRRSRSRQTSSSRNITDDQINDLVSKLQQLLPEIRDRRSDKVSASKVLQETCNYIRSLHREVDDLSERLSELLATTDTAQAAIIRNLLMQ

SEQ ID NO: 109 - protein, 48 aa, glycine max
  peptide (1)..(48): ceres Seq. ID no. 12447178
MIRSMILSPSCNSFFQRFAIGALTRFQLPRCCKRHATILEAYTGKWMT

SEQ ID NO: 110 - DNA, 1750 bp, glycine max
  misc_feature (1)..(1750): ceres Seq. ID no. 12446037
aattttctttcttcaaatctcgcgcgctcctcacttcgagtgttcgactttcctcctccgaccacctcgccgggaattcccgcggcggcgctccttgtctccgcagcttctctctctactgaagccctagagagagaaaatcataaaccctaattcccctgtcaagctcgtagcacagtcacagccatgtatggcggtgatgaagtatcagctatagtaattgacttgggttcacacacttgtaaagctggttatgccggtgaagatgctcccaaggctgtgtttccctctgttgttggagcaattgatcaaatggatgttgatgaagcagataatggtgaaaagaaccctggctctgctccggaatcaaataataataatgttagaaatgctgattctgacaaggccaagggaaagaggaaattgtatgtaggatcccagtccttggggtaccgcagagaccatatggaggtgctgtctccatttaaggatggagttgttgctgactgggatattgttgacagcatatgggatcatgccttcagggactgcctattgattgatcctaaagagcatcctatgctccttgcagagccctcttctaactctcaacaacagagagaaaggacagtggagcatatgtttgagaagtacaaagcacctgcattatttttggcaaaaaatgctgttctgacttcttttgcatcagggcgtgctacgtctgtagttgttgatggtggtggaggctcaactacagttgcaccggtacatgatggttatgttcttcaaaaggctgtggcaacctctccaattggaggagaatttcttacagactgcttgatgaaaagtttggaaagcaagggtattatgataaaacctcggtattccttcaggagaaaggaaatacgccctggagagtttcagacagttgatcttgaatttcccaatactactgaaagctacaaactcttttcccagagggtgattgctagtgatatcaaggaatgtgtgtgccgtgctccagatactccatatgatgagagtgcgtattcaaacattccaatgacctcatatgagcttcctgatggccagacagttgaaattggagctgacagatttaagataccagatgttcttttcaatccatccctggttcagtcaattcctggcatggagagctttgcagaaattgctccttcagttcgtggcctgccccaaatggtgattgaaagcattaataagtgtgatgtagacattcgaagagagttgtttaatagtatactgcttgctggtggtacagcttcaatgcaacaactaaaggaacgccttgagaaagacttgttagaggaatcccctcaagctgccagagtaaaagtattggccagtgggaatgctactgaaagaaggtttagtgtttggatcgggggtagtatattggcatctcttggctccttccagcaaatgtggttctccaaatccgagtatgaagagcatggagcttcatatatccaaaggaagtgtccatgagttgctgtttattttactgccatttatcaggaagtagtactgtgaacgctgatttagtgcttgtctactaatgaatttttatttcctatttaatggccattcccattagatccccattctaactacattattggcctcgaggtgaatcacagcccacctttaactaatctgtattgatgtaaacttgtaaagtgtgcctagagtttgtaacaatgccccgttt

SEQ ID NO: 111 - protein, 446 aa, glycine max
  peptide (1)..(446): ceres Seq. ID no. 12446038
MYGGDEVSAIVIDLGSHTCKAGYAGEDAPKAVFPSVVGAIDQMDVDEADNGEKNPGSAPESNNNNVRNADSDKAKGKRKLYVGSQSLGYRRDHMEVLSPFKDGVVADWDIVDSIWDHAFRDCLLIDPKEHPMLLAEPSSNSQQQRERTVEHMFEKYKAPALFLAKNAVLTSFASGRATSVVVDGGGGSTTVAPVHDGYVLQKAVATSPIGGEFLTDCLMKSLESKGIMIKPRYSFRRKEIRPGEFQTVDLEFPNTTESYKLFSQRVIASDIKECVCRAPDTPYDESAYSNIPMTSYELPDGQTVEIGADRFKIPDVLFNPSLVQSIPGMESFAEIAPSVRGLPQMVIESINKCDVDIRRELFNSILLAGGTASMQQLKERLEKDLLEESPQAARVKVLASGNATERRFSVWIGGSILASLGSFQQMWFSKSEYEEHGASYIQRKCP

SEQ ID NO: 112 - protein, 404 aa, glycine max
  peptide (1)..(404): ceres Seq. ID no. 12446039
MDVDEADNGEKNPGSAPESNNNNVRNADSDKAKGKRKLYVGSQSLGYRRDHMEVLSPFKDGVVADWDIVDSIWDHAFRDCLLIDPKEHPMLLAEPSSNSQQQRERTVEHMFEKYKAPALFLAKNAVLTSFASGRATSVVVDGGGGSTTVAPVHDGYVLQKAVATSPIGGEFLTDCLMKSLESKGIMIKPRYSFRRKEIRPGEFQTVDLEFPNTTESYKLFSQRVIASDIKECVCRAPDTPYDESAYSNIPMTSYELPDGQTVEIGADRFKIPDVLFNPSLVQSIPGMESFAEIAPSVRGLPQMVIESINKCDVDIRRELFNSILLAGGTASMQQLKERLEKDLLEESPQAARVKVLASGNATERRFSVWIGGSILASLGSFQQMWFSKSEYEEHGASYIQRKCP

SEQ ID NO: 113 - protein, 353 aa, glycine max
  peptide (1)..(353): ceres Seq. ID no. 12446040
MEVLSPFKDGVVADWDIVDSIWDHAFRDCLLIDPKEHPMLLAEPSSNSQQQRERTVEHMFEKYKAPALFLAKNAVLTSFASGRATSVVVDGGGGSTTVAPVHDGYVLQKAVATSPIGGEFLTDCLMKSLESKGIMIKPRYSFRRKEIRPGEFQTVDLEFPNTTESYKLFSQRVIASDIKECVCRAPDTPYDESAYSNIPMTSYELPDGQTVEIGADRFKIPDVLFNPSLVQSIPGMESFAEIAPSVRGLPQMVIESINKCDVDIRRELFNSILLAGGTASMQQLKERLEKDLLEESPQAARVKVLASGNATERRFSVWIGGSILASLGSFQQMWFSKSEYEEHGASYIQRKCP

SEQ ID NO: 114 - DNA, 1334 bp, glycine max
  misc_feature (1)..(1334): ceres Seq. ID no. 12430830
atagtgagtgagagagagaaacaaaaagaaaagagagagctctttcattttcattcctttcaagggtttaacacaaacttcttattacttttgaaactatgatagggtttggagggaacagttgcagtgagctttctgagatggacatgacgaacatggaaactaataggaagtttctttctctgcctctcagtaacaacaacaactctggtgataatagaagggtacctgtcactagcaatagcatcgctcaggaacatcactacagccaccaccacaaccccaccgacacttgcagtgtgagggataaaatcatggcacatcctctttttcctcgtctcttatcctcttacctcaattgcctcaaggttggagcacctcctgaagtggtggctagcttagaggagtcatatgcaaaatatgagtccttcaatgcatcatcaggaaggataggaggtggttcaataggtgaagatccagctctggaccagttcatggaggcttactgtgagatgctcattaagtatgagcaagagcttacaaaacccttcaaagaagccatgcttttcttctcaagaattgagtgccagctcaaagccctagcagtttcatcagattttggtcaaagtgaaacatcttcaaagaatgaggttgatgttcatgaaaacaacctagattctcaagctgaagaccgggaactcaaagtccagcttttgcgcaagtatagtggatatctagggagcctcaagaaggaatttttgaagaagaaaaagaatggaaagttgccaaaggaagctcggcaacagctacttgattggtggaacaggcattataaatggccatacccttcggaatctcaaaagcaggcacttgcagaatccacaggcctagatatgaaacagatcaacaactggttcatcaatcagagaaaacgccattggaagccttctgaggatatgcaatttgccgtgatggatgcaactaactactacatggaaaatgttatgtgcaagccatttcccatggatagcatgcctatgcttctttaactcaatcctctaccactagaatatccaaaagctgagccgaaaaccctatctttttttaagttgcctaggtacccaagttgctataatgtcattgatgcatgttttactttaaaattgtagaaagcaggatgaaaattctaccttattatccctgaactgcaatctgtaactctaagtgagcgggaatctaactagctatatactctattatctacactgtttgtgcagatcactagagatgtcgtgtatgtgtagtttcctaatttaatgaatataatttgtttt

SEQ ID NO: 115 - protein, 316 aa, glycine max
  peptide (1)..(316): ceres Seq. ID no. 12430831
MIGFGGNSCSELSEMDMTNMETNRKFLSLPLSNNNNSGDNRRVPVTSNSIAQEHHYSHHHNPTDTCSVRDKIMAHPLFPRLLSSYLNCLKVGAPPEVVASLEESYAKYESFNASSGRIGGGSIGEDPALDQFMEAYCEMLIKYEQELTKPFKEAMLFFSRIECQLKALAVSSDFGQSETSSKNEVDVHENNLDSQAEDRELKVQLLRKYSGYLGSLKKEFLKKKKNGKLPKEARQQLLDWWNRHYKWPYPSESQKQALAESTGLDMKQINNWFINQRKRHWKPSEDMQFAVMDATNYYMENVMCKPFPMDSMPMLL

SEQ ID NO: 116 - protein, 302 aa, glycine max
  peptide (1)..(302): ceres Seq. ID no. 12430832
MDMTNMETNRKFLSLPLSNNNNSGDNRRVPVTSNSIAQEHHYSHHHNPTDTCSVRDKIMAHPLFPRLLSSYLNCLKVGAPPEVVASLEESYAKYESFNASSGRIGGGSIGEDPALDQFMEAYCEMLIKYEQELTKPFKEAMLFFSRIECQLKALAVSSDFGQSETSSKNEVDVHENNLDSQAEDRELKVQLLRKYSGYLGSLKKEFLKKKKNGKLPKEARQQLLDWWNRHYKWPYPSESQKQALAESTGLDMKQINNWFINQRKRHWKPSEDMQFAVMDATNYYMENVMCKPFPMDSMPMLL

SEQ ID NO: 117 - protein, 300 aa, glycine max
  peptide (1)..(300): ceres Seq. ID no. 12430833
MTNMETNRKFLSLPLSNNNNSGDNRRVPVTSNSIAQEHHYSHHHNPTDTCSVRDKIMAHPLFPRLLSSYLNCLKVGAPPEVVASLEESYAKYESFNASSGRIGGGSIGEDPALDQFMEAYCEMLIKYEQELTKPFKEAMLFFSRIECQLKALAVSSDFGQSETSSKNEVDVHENNLDSQAEDRELKVQLLRKYSGYLGSLKKEFLKKKKNGKLPKEARQQLLDWWNRHYKWPYPSESQKQALAESTGLDMKQINNWFINQRKRHWKPSEDMQFAVMDATNYYMENVMCKPFPMDSMPMLL

SEQ ID NO: 118 - DNA, 1426 bp, glycine max
  misc_feature (1135)..(1135): n is a, c, g, or t
  misc_feature (1292)..(1292): n is a, c, g, or t
  misc_feature (1356)..(1356): n is a, c, g, or t
  misc_feature (1)..(1426): ceres Seq. ID no. 12451750
aggcaaaaaggataagacagtgagtgtgtatgttgttgacaagcaaaagccatggagggtagtagttgctctaatgacacttcttatttgttggcttttggagaaaacagtggtgggctatgcccaatgacgatgatgcctttggtaacttcccatcatgcaacaaatcctagtaatcctagtaataatactaataataatgaaaacacaaactgtctcttcattcccaactgcagtaacagttctggaactccttctatcatgctccacaacaacaacaacactgatgatgataacaacaaaaccagcactaacactgggttagggtactatttcatggagagtgaccaccatcaccgcaacaacaacaacaatggaagctcctcctcctcttcctcttctgctgtcaaggccaagatcatggctcatcctcactatcaccgtctcttggcagcttacgtcaattgtcagaaggttggagccccaccggaagtggtggcaaggttagaagaagcatgtgcttctgcagcgacaatggctggtgatgcagcagcagcagctggatcaagctgcataggtgaagatccagctttggatcagttcatggaggcttactgtgagatgctcaccaagtatgagcaagaactctccaaacccttaaaggaagccatgctcttccttcaaaggattgagtgccagttcaaaaatcttacaatttcttccaccgactttgcttgcaacgagggtgctgagaggaatggatcatctgaagaggatgttgatctacacaacatgatagatccccaggcagaggacagggaattaaagggtcagcttttgcgcaagtacagcggatacctgggcagtctgaagcaagaattcatgaagaagaggaagaaaggaaagctacctaaagaagcaaggcaacaattacttgaatggtggagcagacattacaaatggccttacccatccgagtcacagaagctggcccttgcagagtcgacaggtctggatcagaagcaaatcaacaactggtttattaatcaaaggaaacggcactggaagccttcagaggacatgcagtttgtggtgatggatccaagccatccacactattacatggataatgttntgggcaatccatttcccatggatctctcccatccaatgctctagaaaagtatccatcctttgtgggctgataatagattcataagctcgttctgtcacttattaaaaccttaagcaattattaattaatatgctttctagtgctcatataagcattntaagaaatcctagattgctatatactatatttatagtacgcaggtgtatcctgctagcttaaanagacggtcctttgtgtggatcatctagttcaaggagttatgaataaataaaattccataaatttaaagcc

SEQ ID NO: 119 - protein, 375 aa, glycine max
  misc_feature (362)..(362): Xaa can be any naturally occurring amino acid
  peptide (1)..(375): ceres Seq. ID no. 12451751
MEGSSCSNDTSYLLAFGENSGGLCPMTMMPLVTSHHATNPSNPSNNTNNNENTNCLFIPNCSNSSGTPSIMLHNNNNTDDDNNKTSTNTGLGYYFMESDHHHRNNNNNGSSSSSSSSAVKAKIMAHPHYHRLLAAYVNCQKVGAPPEVVARLEEACASAATMAGDAAAAAGSSCIGEDPALDQFMEAYCEMLTKYEQELSKPLKEAMLFLQRIECQFKNLTISSTDFACNEGAERNGSSEEDVDLHNMIDPQAEDRELKGQLLRKYSGYLGSLKQEFMKKRKKGKLPKEARQQLLEWWSRHYKWPYPSESQKLALAESTGLDQKQINNWFINQRKRHWKPSEDMQFVVMDPSHPHYYMDNVXGNPFPMDLSHPML

SEQ ID NO: 120 - protein, 350 aa, glycine max
  misc_feature (337)..(337): Xaa can be any naturally occurring amino acid
  peptide (1)..(350): ceres Seq. ID no. 12451752
MTMMPLVTSHHATNPSNPSNNTNNNENTNCLFIPNCSNSSGTPSIMLHNNNNTDDDNNKTSTNTGLGYYFMESDHHHRNNNNNGSSSSSSSSAVKAKIMAHPHYHRLLAAYVNCQKVGAPPEVVARLEEACASAATMAGDAAAAAGSSCIGEDPALDQFMEAYCEMLTKYEQELSKPLKEAMLFLQRIECQFKNLTISSTDFACNEGAERNGSSEEDVDLHNMIDPQAEDRELKGQLLRKYSGYLGSLKQEFMKKRKKGKLPKEARQQLLEWWSRHYKWPYPSESQKLALAESTGLDQKQINNWFINQRKRHWKPSEDMQFVVMDPSHPHYYMDNVXGNPFPMDLSHPML

SEQ ID NO: 121 - protein, 348 aa, glycine max
  misc_feature (335)..(335): Xaa can be any naturally occurring amino acid
  peptide (1)..(348): ceres Seq. ID no. 12451753
MMPLVTSHHATNPSNPSNNTNNNENTNCLFIPNCSNSSGTPSIMLHNNNNTDDDNNKTSTNTGLGYYFMESDHHHRNNNNNGSSSSSSSSAVKAKIMAHPHYHRLLAAYVNCQKVGAPPEVVARLEEACASAATMAGDAAAAAGSSCIGEDPALDQFMEAYCEMLTKYEQELSKPLKEAMLFLQRIECQFKNLTISSTDFACNEGAERNGSSEEDVDLHNMIDPQAEDRELKGQLLRKYSGYLGSLKQEFMKKRKKGKLPKEARQQLLEWWSRHYKWPYPSESQKLALAESTGLDQKQINNWFINQRKRHWKPSEDMQFVVMDPSHPHYYMDNVXGNPFPMDLSHPML

SEQ ID NO: 122 - DNA, 489 bp, glycine max
  misc_feature (1)..(489): ceres Seq. ID no. 13512808
agtagtggggtttgtccaatgatgatgatgcctttagtgacttctcatcatgttggtcatcatccattaaatcatcctattcttaataatcccaatcctaatgaacacacaaacactctcttccttcccatgccttgtactaataataatcaccacccgaaccgcaataaccacaactccaacgccactgagttagggtatttcatggagatccccaacaacaacaacgatggaagctcctcttccccttcttcagctgtcaaggccaagatcatggcccatcctcactatcaccgtctcttggcagcctacgtcaattgtcaaaaggttggggctccgcctgaagtggtgggaaggttagaagaagcatgtgcatctgctgcagtgataatggcaggtggaacagccagcataggcgaggatccagcgctggatcagttcatggaggcttactgtgagatgctgatcaagtacgagcaagagctctcc

SEQ ID NO: 123 - protein, 163 aa, glycine max
  peptide (1)..(163): ceres Seq. ID no. 13512809
SSGVCPMMMMPLVTSHHVGHHPLNHPILNNPNPNEHTNTLFLPMPCTNNNHHPNRNNHNSNATELGYFMEIPNNNNDGSSSSPSSAVKAKIMAHPHYHRLLAAYVNCQKVGAPPEVVGRLEEACASAAVIMAGGTASIGEDPALDQFMEAYCEMLIKYEQELS

SEQ ID NO: 124 - protein, 157 aa, glycine max
  peptide (1)..(157): ceres Seq. ID no. 13512810
MMMMPLVTSHHVGHHPLNHPILNNPNPNEHTNTLFLPMPCTNNNHHPNRNNHNSNATELGYFMEIPNNNNDGSSSSPSSAVKAKIMAHPHYHRLLAAYVNCQKVGAPPEVVGRLEEACASAAVIMAGGTASIGEDPALDQFMEAYCEMLIKYEQELS

SEQ ID NO: 125 - protein, 156 aa, glycine max
  peptide (1)..(156): ceres Seq. ID no. 13512811
MMMPLVTSHHVGHHPLNHPILNNPNPNEHTNTLFLPMPCTNNNHHPNRNNHNSNATELGYFMEIPNNNNDGSSSSPSSAVKAKIMAHPHYHRLLAAYVNCQKVGAPPEVVGRLEEACASAAVIMAGGTASIGEDPALDQFMEAYCEMLIKYEQELS

SEQ ID NO: 126 - DNA, 713 bp, glycine max
  misc_feature (1)..(713): ceres Seq. ID no. 12438736
atagctagccaagagctaccttaccaaactttccctttccgaggcttaggtcaccgactcaccggacagcaccgcaccgttgtcggatggattgcaagtacctaatctgatcgtcgcctattgaatttcgatccgaagcaattcatttcagataatttgttcgtccccttcgagtttcagaaaaactatatatattggtggacacgacacgacacgacacaacacaatgcaatgggaagctccaaagttcaaaggctttccgggatgcaaaagcaagtacttagcctatacagagggtttctgcgtgcagcacgttccaaatcggaggaagaacggtgcaagatagagtcaattatatcgcaagagtttcggcgtaatgcggaggatgtagatcgcaaaaattttctctacattgagtacttacttcgccgtggcaacaaacaacttgatcagcttaaaaaccctggtactacagggttatcttcgttgcaacttcattactctcacccttcaagtaagggttaaaatgcttgtcattaattattactacagttctgtttcgtgagatattcgcaatttattaagtccaataacaaaatcagttgcttactgtggacttacgtttgattttgtattcccaaggataatcacatgaatttcactttcggtttgtaattttgaaatcttaaaaaactggacccaattttagattt

SEQ ID NO: 127 - protein, 97 aa, glycine max
  peptide (1)..(97): ceres Seq. ID no. 12438737
MGSSKVQRLSGMQKQVLSLYRGFLRAARSKSEEERCKIESIISQEFRRNAEDVDRKNFLYIEYLLRRGNKQLDQLKNPGTTGLSSLQLHYSHPSSKG

SEQ ID NO: 128 - protein, 86 aa, glycine max
  peptide (1)..(86): ceres Seq. ID no. 12438738
MQKQVLSLYRGFLRAARSKSEEERCKIESIISQEFRRNAEDVDRKNFLYIEYLLRRGNKQLDQLKNPGTTGLSSLQLHYSHPSSKG

SEQ ID NO: 129 - DNA, 1441 bp, glycine max
  misc_feature (1007)..(1007): n is a, c, g, or t
  misc_feature (1)..(1441): ceres Seq. ID no. 12456213
atacatagatacatcttatttcattgggaaggaaaactctgagttaagttgcagacacacacgttaaccctgcgtgtagcctctgtgtctccacctgcattcaacacacacagcaacagcaacagcacactgatagtagtgtttgagagagagagagagaatgagattaataagaagcaaagcaacactgttgatgcacagaaggaagaaggtagactacacattcccaaggggaggactctgaaatctgaatctgaatctgattcccaccaccttttacaggctatagagccaagagcccaaaaatccctattgttctgactgactatccatttaggttctttggagttttctacaaataaaggactcagattttttagcttcatttccgggagtactctctggacaattatatgcagtgtgctctttttgagaaaacagtaaggtggggactagcaactaggaacaactgatccaagccaaagaggttggagcacctcctgaagtggtggctagcttagaggagtcatgtgccaaatgtgagtccttcaatgcatcatcaggaaggacaggtggttcaataggtgaagatccagctctggaccagttcatggaggcctactgtgagatgctcattaagtatgagcaagagctcactaaacccttcaaagaagccatgcttttcttctcaagaattgagtgtcaactcaaagccctagcagtttcatcagattttggtcaaagtgaaacatcttcgcagaatgaggttgatgttcatgaaaacaacctagattctcaagctgaagatcgtgaactcaaagtccagcttttgcgcaagtatagtggatatctagggagcctcaagaaggaatttttgaagaagaaaaagaatggaaagttgccaaaggaagctcggcaacagctacttgattggtggaacaggcattataaatggccatacccttcggaatctcaaaagcaggcacttgcagaatccacaggcctanatctgaaacaaatcaacaactggttcatcaatcagagaaaacgtcattggaagccttctgaggatatgcaatttgccgtgatggatgcaactaactactacatggaaaatgttatgtgcaagccatttccaatggatggcatgcctatgcttctttaactcaatcctctactagaatattcaaaagcttagccgagaaaaccctatctattttaagttgcctaggtcgccaggttgctatatataatgtgtcattgatgaatgttttactttaaattgtagaaagcaggatgaaaattctgttatccctgaactgcaatctatgtaagtgagcgtgaatctaactatatgctctattatctacactgtttgtggagatcactagagatgttgtgtatgtgttgtttcctaatttaatgaaaataatttgttttc

SEQ ID NO: 130 - protein, 184 aa, glycine max
  misc_feature (133)..(133): Xaa can be any naturally occurring amino acid
  peptide (1)..(184): ceres Seq. ID no. 12456214
MEAYCEMLIKYEQELTKPFKEAMLFFSRIECQLKALAVSSDFGQSETSSQNEVDVHENNLDSQAEDRELKVQLLRKYSGYLGSLKKEFLKKKKNGKLPKEARQQLLDWWNRHYKWPYPSESQKQALAESTGLXLKQINNWFINQRKRHWKPSEDMQFAVMDATNYYMENVMCKPFPMDGMPMLL

SEQ ID NO: 131 - protein, 178 aa, glycine max
  misc_feature (127)..(127): Xaa can be any naturally occurring amino acid
  peptide (1)..(178): ceres Seq. ID no. 12456215
MLIKYEQELTKPFKEAMLFFSRIECQLKALAVSSDFGQSETSSQNEVDVHENNLDSQAEDRELKVQLLRKYSGYLGSLKKEFLKKKKNGKLPKEARQQLLDWWNRHYKWPYPSESQKQALAESTGLXLKQINNWFINQRKRHWKPSEDMQFAVMDATNYYMENVMCKPFPMDGMPMLL

SEQ ID NO: 132 - protein, 162 aa, glycine max
  misc_feature (111)..(111): Xaa can be any naturally occurring amino acid
  peptide (1)..(162): ceres Seq. ID no. 12456216
MLFFSRIECQLKALAVSSDFGQSETSSQNEVDVHENNLDSQAEDRELKVQLLRKYSGYLGSLKKEFLKKKKNGKLPKEARQQLLDWWNRHYKWPYPSESQKQALAESTGLXLKQINNWFINQRKRHWKPSEDMQFAVMDATNYYMENVMCKPFPMDGMPMLL

SEQ ID NO: 133 - DNA, 524 bp, glycine max
  misc_feature (1)..(524): ceres Seq. ID no. 3459752
atgcattactacttgtcttgatttctttctcgatccccaacatcactagctagctccttgtacacactctacaaccccacctagctacatcacttaattagttttaccaatttcaaattctcacctgtcactagctatatttcataactgatcattaccaactcatcactacatattattggctaggattcaccattagacttaagattagttgatttattacatatataagatgtctagcaggaggtcacggtcaaggcaaacaagtagttcaaggaatatcaccgatgatcagatcaatgatcttgtctctaagttgcaacagcttcttccagagattcgcgataggcgctctgacaaggtttcagcttccaaggtgttgcaagagacatgcaactatattagaagcttacacagggaagtgggtgacctaagcgagcgtttatctgagctcctggatacaactgacacggctcaagctgcaataattagaaatttactgatgcaatagatcggtgcagttg

SEQ ID NO: 134 - protein, 92 aa, glycine max
  peptide (1)..(92): ceres Seq. ID no. 3459753
MSSRRSRSRQTSSSRNITDDQINDLVSKLQQLLPEIRDRRSDKVSASKVLQETCNYIRSLHREVGDLSERLSELLDTTDTAQAAIIRNLLMQ

SEQ ID NO: 135 - protein, 48 aa, glycine max
  peptide (1)..(48): ceres Seq. ID no. 3459754
MIRSMILSLSCNSFFQRFAIGALTRFQLPRCCKRHATILEAYTGKWVT

SEQ ID NO: 136 - DNA, 766 bp, glycine max
  misc_feature (1)..(766): ceres Seq. ID no. 12438885
aagttttcaactcttgtcttctgttgcaaaaaccctaattgcggtgtaaaaccctagaaggaggaagaagaagaggaaggatgagacctttagacgagaaggagacgagcgcggtgttcgagaagctgttcaaattcgtcggaaacaacctgaagaacatcgtggagaacccctcacacgaaggtcctgattcgaaccctggccgctactgcttccgcctccacaagaacaagatcttctacgccagcgagtccctcgtcaaacgcgccaccaacgtcgcgcgccccaacctcgtctccctcggcacctgcatcggcaaatacacccacggcggcagcttccacctcaccgtccaggccctaaacttgctcgccgccaacgccaagcacaaggtctggctcaagccgcagtccgagatgtccttcttgtacggtaaccatgtcttgaagggcgcactgggcaaactctccgaaaacattgctgccggcgacggggttgtcgtgttctccatggccgacgtgccgttgggtttcggtgtcgccgcgaagtccacgctggattgtaggaagctggatccgaacgggattgttgttctgcaccaggccgatgtgggagagtacttgaggatggaggatgagctttgattgatgcattttcttgtaattggttttgatccatcttatgaaattacattatcttctatattatctgtacctcctttgaagttgcctatcttctatattatcttcgacttcgttctcgtttc

SEQ ID NO: 137 - protein, 187 aa, glycine max
  peptide (1)..(187): ceres Seq. ID no. 12438886
MRPLDEKETSAVFEKLFKFVGNNLKNIVENPSHEGPDSNPGRYCFRLHKNKIFYASESLVKRATNVARPNLVSLGTCIGKYTHGGSFHLTVQALNLLAANAKHKVWLKPQSEMSFLYGNHVLKGALGKLSENIAAGDGVVVFSMADVPLGFGVAAKSTLDCRKLDPNGIVVLHQADVGEYLRMEDEL

SEQ ID NO: 138 - DNA, 1799 bp, glycine max
  misc_feature (1)..(1799): ceres Seq. ID no. 13531805
aacccaaccaaactgaaccaaatcatcacacacacatactaagaagcaacaattcttctacaatggatctcctccttctggaaaagaccctcataggtctcttcctcgctgcggtggtcgccatcgccgtctccaccctccgcggccggaaattcaagctcccaccgggcccactccccgtcccaatcttcggcaactggctccaagtcggcgacgacctcaaccaccgcaacctcaccgatttggccaaaaaattcggtgacatcttcctcctccgcatggggcagcgcaacctcgtcgtggtttcttcccctgagctcgccaaagaggttctccacacgcagggcgtggagttcggctcccgcacccgcaacgtcgtcttcgacatcttcaccggaaagggccaagacatggtcttcaccgtctacggcgagcactggcgcaaaatgcgccgcatcatgaccgtccccttcttcaccaacaaggttgtgcaacaataccgccatggatgggaatcggaggctgccgccgtcgtcgaggacgtcaagaaaaaccccgacgccgccgtctccggcaccgtcatccgccgccgccttcagctcatgatgtacaacaacatgtaccgcataatgttcgaccggaggttcgagagcgaggaggatcccatcttccagaggctaagagccttgaacggagagaggagtcgcttggcgcagagctttgagtataactatggtgattttattcccatcttgagacccttcttgaagggttacttgaagatttgcaaggaggtgaaggagacgaggttgaagcttttcaaggattacttcgttgacgagaggaagaagcttggaagcaccaagagcaccaacaacaataatgaacttaaatgcgctattgaccacattttggatgcccagagaaaaggcgagatcaacgaagacaacgtcctctacattgttgaaaacatcaacgttgctgcaattgaaacaactctatggtcgattgagtggggcattgctgagcttgtgaaccacccagagatccagcaaaagttaagggatgagattgacagagttcttggagcagggcaccaagtgactgagccagacatccaaaagctcccatacctccaagcagtggtcaaggaaactcttcgtcttagaatggcaatccctctccttgtaccacacatgaacctccacgacgcaaagcttgggggctatgatatcccagctgagagcaagatcttggtgaatgcatggtggctggccaacaaccctgcacactggaagaagccagaggagttccggcctgagaggttcttcgaggaggagtcgcttgttgaagccaatggcaatgactttaggtaccttccctttggtgttggcagaagaagctgccctggaatcattcttgcattgccaattcttggcatcactttgggacgtttggtccaaaactttgagctcttgcctccccctggccagtcacagattgacactagtgagaaaggagggcaatttagcttgcacatactcaagcattccaccattgtggcaaagccaaggtcattttagactccaccacatgatcatgatcatatgatcaatccccttaattttttcttttttcttttcccttttctttactctgtattgtatcaatgcttgaaaatggggttgttccaaaaatgtcatatatatttggcctgctaatgggtattgtaaatctctgaacttgaagtgatgcacggttttgagatggttttctaataaacctacacttttgtctct

SEQ ID NO: 139 - protein, 526 aa, glycine max
  peptide (1)..(526): ceres Seq. ID no. 13531806
PNQTEPNHHTHILRSNNSSTMDLLLLEKTLIGLFLAAVVAIAVSTLRGRKFKLPPGPLPVPIFGNWLQVGDDLNHRNLTDLAKKFGDIFLLRMGQRNLVVVSSPELAKEVLHTQGVEFGSRTRNVVFDIFTGKGQDMVFTVYGEHWRKMRRIMTVPFFTNKVVQQYRHGWESEAAAVVEDVKKNPDAAVSGTVIRRRLQLMMYNNMYRIMFDRRFESEEDPIFQRLRALNGERSRLAQSFEYNYGDFIPILRPFLKGYLKICKEVKETRLKLFKDYFVDERKKLGSTKSTNNNNELKCAIDHILDAQRKGEINEDNVLYIVENINVAAIETTLWSIEWGIAELVNHPEIQQKLRDEIDRVLGAGHQVTEPDIQKLPYLQAVVKETLRLRMAIPLLVPHMNLHDAKLGGYDIPAESKILVNAWWLANNPAHWKKPEEFRPERFFEEESLVEANGNDFRYLPFGVGRRSCPGIILALPILGITLGRLVQNFELLPPPGQSQIDTSEKGGQFSLHILKHSTIVAKPRSF

SEQ ID NO: 140 - protein, 506 aa, glycine max
  peptide (1)..(506): ceres Seq. ID no. 13531807
MDLLLLEKTLIGLFLAAVVAIAVSTLRGRKFKLPPGPLPVPIFGNWLQVGDDLNHRNLTDLAKKFGDIFLLRMGQRNLVVVSSPELAKEVLHTQGVEFGSRTRNVVFDIFTGKGQDMVFTVYGEHWRKMRRIMTVPFFTNKVVQQYRHGWESEAAAVVEDVKKNPDAAVSGTVIRRRLQLMMYNNMYRIMFDRRFESEEDPIFQRLRALNGERSRLAQSFEYNYGDFIPILRPFLKGYLKICKEVKETRLKLFKDYFVDERKKLGSTKSTNNNNELKCAIDHILDAQRKGEINEDNVLYIVENINVAAIETTLWSIEWGIAELVNHPEIQQKLRDEIDRVLGAGHQVTEPDIQKLPYLQAVVKETLRLRMAIPLLVPHMNLHDAKLGGYDIPAESKILVNAWWLANNPAHWKKPEEFRPERFFEEESLVEANGNDFRYLPFGVGRRSCPGIILALPILGITLGRLVQNFELLPPPGQSQIDTSEKGGQFSLHILKHSTIVAKPRSF

SEQ ID NO: 141 - protein, 434 aa, glycine max
  peptide (1)..(434): ceres Seq. ID no. 13531808
MGQRNLVVVSSPELAKEVLHTQGVEFGSRTRNVVFDIFTGKGQDMVFTVYGEHWRKMRRIMTVPFFTNKVVQQYRHGWESEAAAVVEDVKKNPDAAVSGTVIRRRLQLMMYNNMYRIMFDRRFESEEDPIFQRLRALNGERSRLAQSFEYNYGDFIPILRPFLKGYLKICKEVKETRLKLFKDYFVDERKKLGSTKSTNNNNELKCAIDHILDAQRKGEINEDNVLYIVENINVAAIETTLWSIEWGIAELVNHPEIQQKLRDEIDRVLGAGHQVTEPDIQKLPYLQAVVKETLRLRMAIPLLVPHMNLHDAKLGGYDIPAESKILVNAWWLANNPAHWKKPEEFRPERFFEEESLVEANGNDFRYLPFGVGRRSCPGIILALPILGITLGRLVQNFELLPPPGQSQIDTSEKGGQFSLHILKHSTIVAKPRSF

SEQ ID NO: 142 - DNA, 1352 bp, glycine max
  misc_feature (1)..(1352): ceres Seq. ID no. 13581804
agttcacaccctagagggagtggatggattttgttgtttttcatataagggcaaagaaagatagaggagctagctagctagcgagcttcaaaactcaaaagcactgacacatacaaacaaaaagatctggctttcataaaattgagataaaagtgcaccgtctccgaatcatgtttataatattatcttcattagatcttgatctcatttttcatctgaaactaaaagaatatatatgtgcctttaccctatttagctatatgcacacacaacttgtcacatatcatattttgttcatcaagtttataattccttatctttgaacttacttacatccaagtaaggtctaagctagctggagttgattagatctagctagctagttttcgatctgcttaatttgttttccatgatctcttatatatagtaattttatttccctttaattaattaagaaagagttgaagaagaatgggaagggggaagatcgaaatcaaaaggattgagaacacaacaaatcggcaagtgaccttctgcaagagaagaaatgggcttctgaagaaagcttatgagctgtcagtgctgtgtgatgcagaagttgccctcatcgtcttctccagccgtggccgtctctatgagtattccaacaacaacataagatcaacaatagagaggtacaaaaaggcatgttctgatcactcaagcgcgagcactaccacagaaatcaatgctcagtattatcaacaagaatctgcaaagctgcgacagcaaatacagatgctgcaaaattctaacaggcacctgatgggtgatgccttaagcacactgactgtgaaggaacttaagcagttggagaatagacttgaaagaggaatcactagaatcagatctaagaaacatgagatgctactggctgaaattgaatacttccagaaaagggagattgaactggaaaatgaaaatctttgcctccgaactaagataactgacgtggagaggattcagcaagtaaacatggtttctgggccagaactgaatgccattcaggcattagcttcccgtaacttcttcaatccaaatatgttggaaggtggaactgtttaccctcactcagataagaagattcttcatcttgggtgatcaataatatatgatcttggctagcctatggcatgtctatccaattagccggtggctaaatgcattattattactactcttttggttgctcattaactagaccatgtgatctatatactacttatggattatggatacttatgtatgatgattgtatgatatcataacaatcagttagaaataataattaaaaaatagagttgtgtttgaag

SEQ ID NO: 143 - protein, 222 aa, glycine max
  peptide (1)..(222): ceres Seq. ID no. 13581805
MGRGKIEIKRIENTTNRQVTFCKRRNGLLKKAYELSVLCDAEVALIVFSSRGRLYEYSNNNIRSTIERYKKACSDHSSASTTTEINAQYYQQESAKLRQQIQMLQNSNRHLMGDALSTLTVKELKQLENRLERGITRIRSKKHEMLLAEIEYFQKREIELENENLCLRTKITDVERIQQVNMVSGPELNAIQALASRNFFNPNMLEGGTVYPHSDKKILHLG

SEQ ID NO: 144 - protein, 120 aa, glycine max
  peptide (1)..(120): ceres Seq. ID no. 13581806
MLQNSNRHLMGDALSTLTVKELKQLENRLERGITRIRSKKHEMLLAEIEYFQKREIELENENLCLRTKITDVERIQQVNMVSGPELNAIQALASRNFFNPNMLEGGTVYPHSDKKILHLG

SEQ ID NO: 145 - protein, 111 aa, glycine max
  peptide (1)..(111): ceres Seq. ID no. 13581807
MGDALSTLTVKELKQLENRLERGITRIRSKKHEMLLAEIEYFQKREIELENENLCLRTKITDVERIQQVNMVSGPELNAIQALASRNFFNPNMLEGGTVYPHSDKKILHLG

SEQ ID NO: 146 - DNA, 689 bp, glycine max
  misc_feature (1)..(689): ceres Seq. ID no. 12480555
acatcatccattttcattcttcccctcctactcataatagtgagtttactctcagaatttggagaaagagttctcattcagtagtgtgaggagacgtaaaaagttacggaatcccaaaacaaaaaccgaatggcacctaaaaacttgggcaccaaattaacagcacttgttgtagatgacaatatactcaaccgaaagattcatcaaaatctgttggagagtgttggcgtgaaaaatcaaggagtggaaaatggccaagaagcagtggacattcattgccatgggcaaatatttgacctgattctcatggacatggatatgcccatcatgaatggcattgaggcaacaaaggaacttcgctcaatgggcattggtagcatgattgtcggtgtgtcatcacgctgtacggaagcagaaatacgaaaatttatggaagcggggctgaatgactaccatgagaaacccttgaacaatgctaagcttagttcaattcttgataagatcaaccctagttttactcgtaattagtggaaaaaatttaacgttacccgttccttgtttccaaattccatgtttagtttttcctttttttcttttgttgattctgtagttcttcttttactatggtgaagtctgtgactacgcacgtgagtatcaatgttagacaaaaataaagaaaagacttgagg

SEQ ID NO: 147 - protein, 132 aa, glycine max
  peptide (1)..(132): ceres Seq. ID no. 12480556
MAPKNLGTKLTALVVDDNILNRKIHQNLLESVGVKNQGVENGQEAVDIHCHGQIFDLILMDMDMPIMNGIEATKELRSMGIGSMIVGVSSRCTEAEIRKFMEAGLNDYHEKPLNNAKLSSILDKINPSFTRN

SEQ ID NO: 148 - protein, 73 aa, glycine max
  peptide (1)..(73): ceres Seq. ID no. 12480557
MDMDMPIMNGIEATKELRSMGIGSMIVGVSSRCTEAEIRKFMEAGLNDYHEKPLNNAKLSSILDKINPSFTRN

SEQ ID NO: 149 - protein, 71 aa, glycine max
  peptide (1)..(71): ceres Seq. ID no. 12480558
MDMPIMNGIEATKELRSMGIGSMIVGVSSRCTEAEIRKFMEAGLNDYHEKPLNNAKLSSILDKINPSFTRN

SEQ ID NO: 150 - DNA, 944 bp, glycine max
  misc_feature (1)..(944): ceres Seq. ID no. 12610129
taaaaccctagccgccgtccgtccccaaccttcaccgccgctccccctcaaccctccgccccccaaaccccgccgccgcctccgccatgcggccgctcgacgagaaggagaccacgatggtcttcgagaagctgttcaagttcaccggccccaacctgaagcacctcctggagcggccctccgtggagggccccgacccggagcccggccgctactgcctccgcctccacaagaaccgcgtcttctacgcctccgagtcgctcgtccgccgcgccaccgccgtgtcccgcacgcgcctcgccggggtcggcacgcccatcggcaagttcacccacggcggcgccttccacctcaccgtccacgcgctcgacctcctcgccgcccacgcgcgccgccgcatctggctcaagcccgacacggagcgctcgttcctcttcggcaactccgtgcccaagtccgcgctcgcgcgcatcacggagaacaccaagtccggcgatggcgtcgtcgtcatgtccatggccgacgtgccgctcgggttcggggtcgccgccaggggcgcgcaggactgcaggaaggccgacaccaacgccgtggtggtgctccaccagtccgacgccggcgagtacctccgcaaggaggaggagctcatgtgaggtgatgtgtgactgttctgaaagatgtgttccctcccttggttgtgatcaccgatcatctgtgcttgcaatcagtgtgcagttcgtcgaccattctgtattctgtagttttcatacagggcaaggaaaagcggatgtctgatctatctgagtgtcatagtagagtagttctcttttcaggcagttctgatggctataatttttgtcacgccgtagcttatcagtaagctactgcattgtatcgtgtgaactgtgaactgaagattccgtgaactaaatattatgataatg

SEQ ID NO: 151 - protein, 218 aa, glycine max
  peptide (1)..(218): ceres Seq. ID no. 12610130
KTLAAVRPQPSPPLPLNPPPPKPRRRLRHAAARREGDHDGLREAVQVHRPQPEAPPGAALRGGPRPGARPLLPPPPQEPRLLRLRVARPPRHRRVPHAPRRGRHAHRQVHPRRRLPPHRPRARPPRRPRAPPHLAQARHGALVPLRQLRAQVRARAHHGEHQVRRWRRRHVHGRRAARVRGRRQGRAGLQEGRHQRRGGAPPVRRRRVPPQGGGAHVR

SEQ ID NO: 152 - protein, 188 aa, glycine max
  peptide (1)..(188): ceres Seq. ID no. 12610131
MRPLDEKETTMVFEKLFKFTGPNLKHLLERPSVEGPDPEPGRYCLRLHKNRVFYASESLVRRATAVSRTRLAGVGTPIGKFTHGGAFHLTVHALDLLAAHARRRIWLKPDTERSFLFGNSVPKSALARITENTKSGDGVVVMSMADVPLGFGVAARGAQDCRKADTNAVVVLHQSDAGEYLRKEEELM

SEQ ID NO: 153 - protein, 178 aa, glycine max
  peptide (1)..(178): ceres Seq. ID no. 12610132
MVFEKLFKFTGPNLKHLLERPSVEGPDPEPGRYCLRLHKNRVFYASESLVRRATAVSRTRLAGVGTPIGKFTHGGAFHLTVHALDLLAAHARRRIWLKPDTERSFLFGNSVPKSALARITENTKSGDGVVVMSMADVPLGFGVAARGAQDCRKADTNAVVVLHQSDAGEYLRKEEELM

SEQ ID NO: 154 - DNA, 1002 bp, glycine max
  misc_feature (1)..(1002): ceres Seq. ID no. 13505209
cccgcgcgccgattccactaggttttccaactctcctgcacccgccgccgccacctcccctcccccaccacaccctcagccatggcggtcgtggagccggtgcccgggggcgacgtgaagctcttcaaccgctggaccttcgaggatgttcaggtgaccgacatctcgctgaacgactacctcgcggtgacgtcgatgaagcactacacgttcctgccgcactcggccggccgctactcggccaagcgcttccgcaaggcgcagtgccccatcatcgagcgcctcaccaactccctcatgatgcacgggcgcaacaacgggaagaagatcatggccgtccgcatcatcaagcacaccatggagatcatccacctcctcaccgacgccaaccccatccagatcatcgttgatgccatcatcaacagtggtccacgtgaggatgctacccgtattgggtctgctggtgttgtgaggagacaggctgtggatatctctcccctcaggagggtgaaccaggccatctaccttctgactactggtgccagggagagcgccttcaggaacgtcaagaccattgctgagtgccttgctgatgagctgatcaatgctgccaagggctcatccaacagctatgccatcaagaagaaggacgagattgagcgtgttgccaaggctaaccgttaagatggatgtgcatctgagggtctcgcttttggctggagattttgaagtgaatgtgttttacaagatatctagaactgttttagctatgcgttaagttttatggaatgaacatttcgatgtagtggcaagaacgtgcctgatgcttgtctttgtgaaacctattcctgttcagacatatttggatatgttgaatgtcgatgtgttctgtcataatatgtcttggtttgatctattcttgcgaatgcttattccactttgcaatagtgtatgcatgcttactattgcatacttaagctctgtcgtacttgtcattacaac

SEQ ID NO: 155 - protein, 200 aa, glycine max
  peptide (1)..(200): ceres Seq. ID no. 13505210
MAVVEPVPGGDVKLFNRWTFEDVQVTDISLNDYLAVTSMKHYTFLPHSAGRYSAKRFRKAQCPIIERLTNSLMMHGRNNGKKIMAVRIIKHTMEIIHLLTDANPIQIIVDAIINSGPREDATRIGSAGVVRRQAVDISPLRRVNQAIYLLTTGARESAFRNVKTIAECLADELINAAKGSSNSYAIKKKDEIERVAKANR

SEQ ID NO: 156 - protein, 162 aa, glycine max
  peptide (1)..(162): ceres Seq. ID no. 13505211
MKHYTFLPHSAGRYSAKRFRKAQCPIIERLTNSLMMHGRNNGKKIMAVRIIKHTMEIIHLLTDANPIQIIVDAIINSGPREDATRIGSAGVVRRQAVDISPLRRVNQAIYLLTTGARESAFRNVKTIAECLADELINAAKGSSNSYAIKKKDEIERVAKANR

SEQ ID NO: 157 - protein, 135 aa, glycine max
  peptide (1)..(135): ceres Seq. ID no. 13505212
RAPIPLGFPTLLHPPPPPPLPHHTLSHGGRGAGARGRREALQPLDLRGCSGDRHLAERLPRGDVDEALHVPAALGRPLLGQALPQGAVPHHRAPHQLPHDARAQQREEDHGRPHHQAHHGDHPPPHRRQPHPDHR

SEQ ID NO: 158 - DNA, 457 bp, glycine max
  misc_feature (1)..(457): ceres Seq. ID no. 12484682
aactcacccttttctgttgtaaaaccctaattgcggtgtaaaaccctagaaggaggaggaggaaggatgagacccttggacgagaaggagacgagcgcagtgttcgagaagctgttcaagttcgtcggaaacaacctgaagaacatcgtggagaacccctcgcacgaaggtcccgattcgaaccccgggcgctactgcttccggctccacaagaacaagatcttctacgccagcgagtccctcgtgaagcgcgccaccaacgtcgcccgccccaacctggtctccctcggcacctgcatcgggaaatacacccacggcggcagcttccacctcaccgtccaggccctcaacttgctcgccgccaacgccaagcacaaggtctggctcaagccccagtccgagatgtccttcttgtacggcaaccatgtcttgaagggcgctctcgggaaactctccg

SEQ ID NO: 159 - protein, 130 aa, glycine max
  peptide (1)..(130): ceres Seq. ID no. 12484683
MRPLDEKETSAVFEKLFKFVGNNLKNIVENPSHEGPDSNPGRYCFRLHKNKIFYASESLVKRATNVARPNLVSLGTCIGKYTHGGSFHLTVQALNLLAANAKHKVWLKPQSEMSFLYGNHVLKGALGKLS

SEQ ID NO: 160 - DNA, 562 bp, glycine max
  misc_feature (1)..(562): ceres Seq. ID no. 13645433
taagtcaccacactccgtttcccgccgcctccgccctctgccgccgcagccgcagccatgggcgcgtacaagttcgtgtcggagctatggaggaggaagcagtccgacgtgatgaggttcgtgcagcgcgtgcgttgctgggagtacaggcagcagcccgccatcgtccgcatcaccaggcccacccgccccgacagggcacgccgtctcggcttcaaggccaagcaggggtatgtggtctaccgtatccgtgtgagacgtggtggcaggaagcgcccagtgcccaagggtattgtttatggcaagcctaagcaccagggtattacccaactcaagttccagaggaacaagaggtctgttgctgaggaaagagctgggcgcaggcttggtggtcttcgtgtcctcaactcctactgggtgaatgaggactccacctacaagtactttgaggtcatccttgttgacgttgctcacagcgctgtccgcaacgatccaaggatcaactggctctgcaagcctgtgcacaagcaccgtgagctgcgtggtctcacctctgcgggaa

SEQ ID NO: 161 - protein, 168 aa, glycine max
  peptide (1)..(168): ceres Seq. ID no. 13645434
MGAYKFVSELWRRKQSDVMRFVQRVRCWEYRQQPAIVRITRPTRPDRARRLGFKAKQGYVVYRIRVRRGGRKRPVPKGIVYGKPKHQGITQLKFQRNKRSVAEERAGRRLGGLRVLNSYWVNEDSTYKYFEVILVDVAHSAVRNDPRINWLCKPVHKHRELRGLTSAG

SEQ ID NO: 162 - protein, 150 aa, glycine max
  peptide (1)..(150): ceres Seq. ID no. 13645435
MRFVQRVRCWEYRQQPAIVRITRPTRPDRARRLGFKAKQGYVVYRIRVRRGGRKRPVPKGIVYGKPKHQGITQLKFQRNKRSVAEERAGRRLGGLRVLNSYWVNEDSTYKYFEVILVDVAHSAVRNDPRINWLCKPVHKHRELRGLTSAG

SEQ ID NO: 163 - protein, 102 aa, glycine max
  peptide (1)..(102): ceres Seq. ID no. 13645436
SHHTPFPAASALCRRSRSHGRVQVRVGAMEEEAVRRDEVRAARALLGVQAAARHRPHHQAHPPRQGTPSRLQGQAGVCGLPYPCETWWQEAPSAQGYCLWQA

SEQ ID NO: 164 - DNA, 904 bp, triticum aestivum
  misc_feature (529)..(529): n is a, c, g, or t
  misc_feature (612)..(612): n is a, c, g, or t
  misc_feature (618)..(618): n is a, c, g, or t
  misc_feature (754)..(754): n is a, c, g, or t
  misc_feature (1)..(904): ceres Seq. ID no. 13585369
cctaatcgaaaaaaaaccctaagtcaccacactccgtttcccgccgcctccgccctctgccgccgcagccgcagccatgggcgcgtacaagttcgtgtcggagctatggaggaggaagcagtccgacgtgatgaggttcgtgcagcgcgtgcgttgctgggagtacaggcagcagcccgccatcgtccgcatcaccaggcccacccgccccgacagggcacgccgtctcggcttcaaggccaaacagggttatgtgatctaccgtatccgtgtgagacgtggtggcaggaagcgcccagtgcccaagggtattgtttatggcaagcccaagcaccagggtattacccaactcaagttccagaggaacaagaggtctgttgctgaggaaagagctgggcgcaggcttggtggtcttcgtgtcctcaactcctactgggtgaatgaggactccacctacaagtactttgaggtcatccttgttgacgttgctcacagcgctgtccgcaacgatccaaggatcaactggctntgcaagcctgtgcacaagcaccgtgagctgcgtggtctcacctctgcgggaaagaaattccgtggtctgcgtggcaagggtcnccgccnccacaagaacaggccctcaaggagagccacctggaagaggaaccagaccgtctccctccgccgctaccgttaagcttatgtgctgttatgttctgtggaacctgccctatgcttaccttgatagtatctgtttgcnccgtcaaattttgctaccttcgttctgtggctgaacctccaggcatgtctgttgaactatttgctatgtgcctgtgttacctggaattcgaatgattgaagtagtcctcgatgtgtgctgattattgttttaatgctttgatgttttttt

SEQ ID NO: 165 - protein, 204 aa, triticum aestivum
  misc_feature (151)..(151): Xaa can be any naturally occurring amino acid
  misc_feature (179)..(179): Xaa can be any naturally occurring amino acid
  misc_feature (181)..(181): Xaa can be any naturally occurring amino acid
  peptide (1)..(204): ceres Seq. ID no. 13585370
MGAYKFVSELWRRKQSDVMRFVQRVRCWEYRQQPAIVRITRPTRPDRARRLGFKAKQGYVIYRIRVRRGGRKRPVPKGIVYGKPKHQGITQLKFQRNKRSVAEERAGRRLGGLRVLNSYWVNEDSTYKYFEVILVDVAHSAVRNDPRINWXCKPVHKHRELRGLTSAGKKFRGLRGKGXRXHKNRPSRRATWKRNQTVSLRRYR

SEQ ID NO: 166 - protein, 186 aa, triticum aestivum
  misc_feature (133)..(133): Xaa can be any naturally occurring amino acid
  misc_feature (161)..(161): Xaa can be any naturally occurring amino acid
  misc_feature (163)..(163): Xaa can be any naturally occurring amino acid
  peptide (1)..(186): ceres Seq. ID no. 13585371
MRFVQRVRCWEYRQQPAIVRITRPTRPDRARRLGFKAKQGYVIYRIRVRRGGRKRPVPKGIVYGKPKHQGITQLKFQRNKRSVAEERAGRRLGGLRVLNSYWVNEDSTYKYFEVILVDVAHSAVRNDPRINWXCKPVHKHRELRGLTSAGKKFRGLRGKGXRXHKNRPSRRATWKRNQTVSLRRYR

SEQ ID NO: 167 - protein, 127 aa, triticum aestivum
  peptide (1)..(127): ceres Seq. ID no. 13585372
PNRKKTLSHHTPFPAASALCRRSRSHGRVQVRVGAMEEEAVRRDEVRAARALLGVQAAARHRPHHQAHPPRQGTPSRLQGQTGLCDLPYPCETWWQEAPSAQGYCLWQAQAPGYYPTQVPEEQEVCC

SEQ ID NO: 168 - DNA, 701 bp, triticum aestivum
  misc_feature (622)..(622): n is a, c, g, or t
  misc_feature (1)..(701): ceres Seq. ID no. 12443604
atgcacaacttgtcttccctctcttccaacaccacttcttcttagttcctctccgtccctgtctgccaccacttctgtctctcaaacttgtctcactccaaccataaaccctcactgtcttgggctctctctgccaagcatccaattcctaagtacatccgatcactcacatttgcagtgatgtcgagccgtaggtcaaggtcaaggcagtccggctcgtcgaggatcaccgacgagcaaatcagcgaccttgtctccaagttgcaggacctccttcccgaggcgcgtctccggggcaatgatagagtgccatcttcaagggtgctgcaggagacgtgcacctacatcaggagcttgcaccgggaggtggacgacctgagcgagaggctgtcggagctgctggcgacctcggacatgagcagcgcgcaagcggccatcatccgcagcttgctgatgtagagccggctcccatgcagtgcgcaggcgcctcgtcgctgtcttgctgagcgcacaagcctgaatttgagcgtttgtagcctagggagcgatctttaattagtaccggagttgcaggttacctacttaatccgcgtgtgtgcgctgtcgtcgtgtcatcatcgtnttaatcaggcccatcttttttgtgtgtgtacttaaatcaagtcgttaaatcaaaccccgcccgtggttggtgcaagttg

SEQ ID NO: 169 - protein, 92 aa, triticum aestivum
  peptide (1)..(92): ceres Seq. ID no. 12443605
MSSRRSRSRQSGSSRITDEQISDLVSKLQDLLPEARLRGNDRVPSSRVLQETCTYIRSLHREVDDLSERLSELLATSDMSSAQAAIIRSLLM

SEQ ID NO: 170 - protein, 59 aa, triticum aestivum
  peptide (1)..(59): ceres Seq. ID no. 12443606
CTTCLPSLPTPLLLSSSPSLSATTSVSQTCLTPTINPHCLGLSLPSIQFLSTSDHSHLQ

SEQ ID NO: 171 - DNA, 698 bp, triticum aestivum
  misc_feature (526)..(526): n is a, c, g, or t
  misc_feature (563)..(563): n is a, c, g, or t
  misc_feature (572)..(572): n is a, c, g, or t
  misc_feature (608)..(608): n is a, c, g, or t
  misc_feature (649)..(650): n is a, c, g, or t
  misc_feature (1)..(698): ceres Seq. ID no. 11421342
tttctgctgtgcatgatggttatgttttgcaaaagtctgtggcaacttctccgataggcggtgaatttttgactgactgtatgatgaaaagcttggagagcaagggcgttgttatcaggccccggtattccttcaagaagaaggaagtgagccctggagattataaggttgtagaccttgattttccgaacaccacggatagttacaggttgtaccacatgagagctattgctagcgacatcaaggagacggtttgtagagttccagatactcccttcgatgaagtggcatatgcaaatgttcccacaacttcatacgagcttccagacgggcaaactattgaagttggtgcagagagattcaagattcctgacattttgttcaacccattcctttctcagactattcctgggatcgatggatttggagattccacttcgattcgtggacttccacgaatggccttgaaagtgtaaataggtgtgatgttgcattcgcaaggagctactcagcagcatcttgcttntggtggttatcatcaattctgcagtaaaggccgctanaaaagaagnctggggagccctcaaatctcggtaaggtttggcagnggaaatcacgaaggcattcatgctggttggaggacatcttnntcctggcgtccacaatgggttcaggaaaacaaaccgggttctttccag

SEQ ID NO: 172 - protein, 157 aa, triticum aestivum
  peptide (1)..(157): ceres Seq. ID no. 11421343
SAVHDGYVLQKSVATSPIGGEFLTDCMMKSLESKGVVIRPRYSFKKKEVSPGDYKVVDLDFPNTTDSYRLYHMRAIASDIKETVCRVPDTPFDEVAYANVPTTSYELPDGQTIEVGAERFKIPDILFNPFLSQTIPGIDGFGDSTSIRGLPRMALKV

SEQ ID NO: 173 - protein, 131 aa, triticum aestivum
  peptide (1)..(131): ceres Seq. ID no. 11421344
MMKSLESKGVVIRPRYSFKKKEVSPGDYKVVDLDFPNTTDSYRLYHMRAIASDIKETVCRVPDTPFDEVAYANVPTTSYELPDGQTIEVGAERFKIPDILFNPFLSQTIPGIDGFGDSTSIRGLPRMALKV

SEQ ID NO: 174 - protein, 130 aa, triticum aestivum
  peptide (1)..(130): ceres Seq. ID no. 11421345
MKSLESKGVVIRPRYSFKKKEVSPGDYKVVDLDFPNTTDSYRLYHMRAIASDIKETVCRVPDTPFDEVAYANVPTTSYELPDGQTIEVGAERFKIPDILFNPFLSQTIPGIDGFGDSTSIRGLPRMALKV

SEQ ID NO: 175 - DNA, 452 bp, triticum aestivum
  misc_feature (1)..(452): ceres Seq. ID no. 4390460
cacgcgcctcgccggggtcggcacgcccatcggcaagttcacccacggcggcgccttccacctcaccgtccacgcgctcgacctcctcgccgcccacgcgcgccgccgcatctggctcaagcccgacacggagcgctcgttcctcttcggcaactccgtgcccaagtccgcgctcgcgcgcatcaccgagaacaccaagtccggcgacggcgtcgtcgtcatgtccatggccgacgtgccgctcgggttcggggtcgccgccaggggcgcgcaggactgcaggaaggccgataccaacgccgtggtggtgctccaccagtcggatgctggcgagtacctccgcaaggaggaggagctcatgtgaggtgagggtcgaggcttggttgttattgtgtttactgattggaccttatgcgtgaatttagggctggagcagatattgccaatatctg

SEQ ID NO: 176 - protein, 138 aa, triticum aestivum
  peptide (1)..(138): ceres Seq. ID no. 4390461
RASPGSARPSASSPTAAPSTSPSTRSTSSPPTRAAASGSSPTRSARSSSATPCPSPRSRASPRTPSPATASSSCPWPTCRSGSGSPPGARRTAGRPIPTPWWCSTSRMLASTSARRRSSCEVRVEAWLLLCLLIGPYA

SEQ ID NO: 177 - protein, 122 aa, triticum aestivum
  peptide (1)..(122): ceres Seq. ID no. 4390462
HAPRRGRHAHRQVHPRRRLPPHRPRARPPRRPRAPPHLAQARHGALVPLRQLRAQVRARAHHREHQVRRRRRRHVHGRRAARVRGRRQGRAGLQEGRYQRRGGAPPVGCWRVPPQGGGAHVR

SEQ ID NO: 178 - protein, 120 aa, triticum aestivum
  peptide (1)..(120): ceres Seq. ID no. 4390463
TRLAGVGTPIGKFTHGGAFHLTVHALDLLAAHARRRIWLKPDTERSFLFGNSVPKSALARITENTKSGDGVVVMSMADVPLGFGVAARGAQDCRKADTNAVVVLHQSDAGEYLRKEEELM

SEQ ID NO: 179 - DNA, 1161 bp, triticum aestivum
  misc_feature (1)..(1161): ceres Seq. ID no. 12598265
agacacgagcggagcaccgagcaggcgcgcagcacatcatctgtcacaccggcggcggcggcggcatggggatgggcggtcgaggtgtggttggcgacaggtggtcgcagcgcgtcctctggatgtgcgccatcggcagcgccgtgagcctgtacttcgtggcggtggagaggcaggcgcagaaccgcgcgcgggcggtgtccgagggctttaagggcctggacggcgcagacggaggtggcagccgtggcggggaggacgtgtgactttggcggcgccgattccactaggttttccagctctcccgcacccgccgccgccacctccccactccccaccaccccctcagccatggcggtcgtcgagccggtgcccgggggcgacgtgaagctattcaaccgctggaccttcgaggatgttcaggtgagcgacatctcgctgaacgactacctcgcggtgacgtcgatgaagcactacaccttcctgccgcactcggccggccgctactcggccaagcgcttccgcaaggcgcagtgccccatcatcgagcgcctcaccaactccctcatgatgcacgggcgcaacaacggtaagaagatcatggccgtccgcatcatcaagcacaccatggagatcatccacctcctcaccgacgccaaccccatccagatcatcgttgacgccatcatcaacagtggtccacgtgaggatgctacccgtattgggtctgctggtgttgtgaggaggcaggctgtggatatctcccccctgaggagggtgaaccaggccatctaccttctgaccactggtgccagggagagcgccttcaggaatgtcaagaccattgctgagtgcctcgctgatgagctgatcaatgctgccaagggctcatccaacagctatgccatcaagaagaaggacgagattgagcgtgttgccaaggctaaccgttaagatggatgtgcatctgagggtctcatgcttttggctggagattttggactgaatgtgttttacaagatatctagaactgttttagctatgctttaagtttgtgatgaaagaacatttcgatgtagcggcaaggacttgcctgtctttatgaaaccaattcctgttcagacaaatttggagatgttgaatgtgtcgttgtgttctctc

SEQ ID NO: 180 - protein, 200 aa, triticum aestivum
  peptide (1)..(200): ceres Seq. ID no. 12598266
MAVVEPVPGGDVKLFNRWTFEDVQVSDISLNDYLAVTSMKHYTFLPHSAGRYSAKRFRKAQCPIIERLTNSLMMHGRNNGKKIMAVRIIKHTMEIIHLLTDANPIQIIVDAIINSGPREDATRIGSAGVVRRQAVDISPLRRVNQAIYLLTTGARESAFRNVKTIAECLADELINAAKGSSNSYAIKKKDEIERVAKANR

SEQ ID NO: 181 - protein, 162 aa, triticum aestivum; identical to SEQ ID NO: 156
  peptide (1)..(162): ceres Seq. ID no. 12598267

SEQ ID NO: 182 - protein, 128 aa, triticum aestivum
  peptide (1)..(128): ceres Seq. ID no. 12598268
MMHGRNNGKKIMAVRIIKHTMEIIHLLTDANPIQIIVDAIINSGPREDATRIGSAGVVRRQAVDISPLRRVNQAIYLLTTGARESAFRNVKTIAECLADELINAAKGSSNSYAIKKKDEIERVAKANR

SEQ ID NO: 183 - DNA, 464 bp, triticum aestivum
  misc_feature (1)..(464): ceres Seq. ID no. 13502974
aactcatctcaccctttctcttcttctgctaagttccgggctgagactaagaagaggtcgagatggggaggggaaggattgagatcaagaggatcgagaacaccactagccggcaggtgaccttctgcaagcgccggaacgggctcctcaaaaaggcctacgagctctccgtcctgtgcgacgccgaggtggcgctcatcgtcttctccagccgcggccgcctctacgagtactccaacaacagtgtcaaggctacaattgacaggtacaagaaggcacatgcttgtggctcaacctctggggtacctctcatagaagtcaatgctcagcaatactaccagcaagaagctgcaaagctgcgccaccagatccagatgctgcaaagcactaacaagcacttggttggtgattctgttgggaacctgtcactcaaggagctgaagcaactcgaaagccggcttgag

SEQ ID NO: 184 - protein, 134 aa, triticum aestivum
  peptide (1)..(134): ceres Seq. ID no. 13502975
MGRGRIEIKRIENTTSRQVTFCKRRNGLLKKAYELSVLCDAEVALIVFSSRGRLYEYSNNSVKATIDRYKKAHACGSTSGVPLIEVNAQQYYQQEAAKLRHQIQMLQSTNKHLVGDSVGNLSLKELKQLESRLE

SEQ ID NO: 185 - DNA, 492 bp, triticum aestivum
  misc_feature (1)..(492): ceres Seq. ID no. 11429655
ttgtagagttccagatactcccttcgatgaagtggcatatgcaaatgttcccacaacttcatacgagcttccagacgggcaaactattgaagttggtgcagagagattcaagattcctgacattttgttcaacccattcctttctcagactattcctgggatcgatggatttggagattccacttcgattcgtggacttccacgaatggtccttgaaagtgtaaataggtgtgatgttgacattcgcaaggagctactcagcagcatcttgctttctggtggctcatcatcaattctgcagttaaaggaccgtctagaaaaagaagtactggaggagtcccctcaaaatgctcgggtaaaggttttggcaagtggaaattcaacggagaggcgattcagtgtctggattggagggagcatccttgcatcccttgggtcgttccagcaaatgtggttctccaaggcagagtacgaagagcacggcgttcctat

SEQ ID NO: 186 - protein, 163 aa, triticum aestivum
  peptide (1)..(163): ceres Seq. ID no. 11429656
CRVPDTPFDEVAYANVPTTSYELPDGQTIEVGAERFKIPDILFNPFLSQTIPGIDGFGDSTSIRGLPRMVLESVNRCDVDIRKELLSSILLSGGSSSILQLKDRLEKEVLEESPQNARVKVLASGNSTERRFSVWIGGSILASLGSFQQMWFSKAEYEEHGVP

SEQ ID NO: 187 - protein, 95 aa, triticum aestivum
  peptide (1)..(95): ceres Seq. ID no. 11429657
MVLESVNRCDVDIRKELLSSILLSGGSSSILQLKDRLEKEVLEESPQNARVKVLASGNSTERRFSVWIGGSILASLGSFQQMWFSKAEYEEHGVP

SEQ ID NO: 188 - DNA, 679 bp, Zea mays subsp. mays
  misc_feature (1)..(679): ceres Seq. ID no. 12401193
agtcgtcaggcgctctgcgctcaggttttcatcttcgggtgcccttcaccggcgaccagcacccaacaggctttgatgattgctgtacaaggatgatatggcctcccgcccaaagctgtctggtattcagaagcaggttttggcgctgtacaggggattccttcggacagcacgccttaaggcccctgaagaacgccgcaggatcgagtctgttgtctttgcagagttccgtgacaacgcaaggagtgttgaccgtaggaactttgtacacatagagtacttgttgaggagagggaagaagcagcttgagcagctcaagaatcctgatattactggactcgctacccttgtagtaaagaaatgagcaacagttgtcgaaatttacaggtccattcattgccagaacaaagtatcatcatgataccggtgtttctaatgtgaagatttggaacctgccactgatggtgtccaaataaaataaatatgaccacagattacagatttgatatccagggactttctaatcagcctattagaatctgaagataaatcttatagcacctgtccggtgacccatagtaatattattttactcttgctgtaagcagaggttaacactaaaaaaagtattgtgattggtctgaactttttgtgcggtataaagcatgtgattcagccc

SEQ ID NO: 189 - protein, 90 aa, Zea mays subsp. mays
  peptide (1)..(90): ceres Seq. ID no. 12401194
SSGALRSGFHLRVPFTGDQHPTGFDDCCTRMIWPPAQSCLVFRSRFWRCTGDSFGQHALRPLKNAAGSSLLSLQSSVTTQGVLTVGTLYT

SEQ ID NO: 190 - protein, 88 aa, Zea mays subsp. mays
  peptide (1)..(88): ceres Seq. ID no. 12401195
MASRPKLSGIQKQVLALYRGFLRTARLKAPEERRRIESVVFAEFRDNARSVDRRNFVHIEYLLRRGKKQLEQLKNPDITGLATLVVKK

SEQ ID NO: 191 - protein, 60 aa, Zea mays subsp. mays
  peptide (1)..(60): ceres Seq. ID no. 12401196
MIWPPAQSCLVFRSRFWRCTGDSFGQHALRPLKNAAGSSLLSLQSSVTTQGVLTVGTLYT

SEQ ID NO: 192 - DNA, 467 bp, Zea mays subsp. mays
  misc_feature (357)..(357): n is a, c, g, or t
  misc_feature (411)..(411): n is a, c, g, or t
  misc_feature (431)..(431): n is a, c, g, or t
  misc_feature (1)..(467): ceres Seq. ID no. 1423353
atagcttggctcgcacggtgtcgctggctcccctccccttccctccccactgttacatccagcgagctgttcacggccgtgcsaagctcagtgaggcagcgtttgckagtccgggacgaggggagcggcgggcgacatggggaggggccgcagcgagataaagcggatcgagaaccccacgcagcggcagtccaccttctacaagcgcagggacggcctcttcaagaaggctagggagctctccgttctgtgcgacgtggacctgctgctgctcctcttctccacctccggcaagctctaccaggccacgacgcacaccaaggtctggaccgacatccgccaggagcggcgcgcggnastggagaaggcggacagatgtgcgagctgatggagaaggagctgcgggtcatnacgggtggacgacgggggancagtacacggtgccgtcgctggagctgctggagcac

SEQ ID NO: 193 - protein, 77 aa, Zea mays subsp. mays
  misc_feature (28)..(28): Xaa can be any naturally occurring amino acid
  misc_feature (36)..(36): Xaa can be any naturally occurring amino acid
  peptide (1)..(77): ceres Seq. ID no. 1423354
IAWLARCRWLPSPSLPTVTSSELFTAVXSSVRQRLXVRDEGSGGRHGEGPQRDKADREPHAAAVHLLQAQGRPLQEG

SEQ ID NO: 194 - protein, 52 aa, Zea mays subsp. mays
  misc_feature (27)..(27): Xaa can be any naturally occurring amino acid
  misc_feature (35)..(35): Xaa can be any naturally occurring amino acid
  peptide (1)..(52): ceres Seq. ID no. 1423355
SLARTVSLAPLPFPPHCYIQRAVHGRXKLSEAAFXSPGRGERRATWGGAAAR

SEQ ID NO: 195 - protein, 83 aa, Zea mays subsp. mays
  misc_feature (74)..(75): Xaa can be any naturally occurring amino acid
  peptide (1)..(83): ceres Seq. ID no. 1423356
MGRGRSEIKRIENPTQRQSTFYKRRDGLFKKARELSVLCDVDLLLLLFSTSGKLYQATTHTKVWTDIRQERRAXXEKADRCAS

SEQ ID NO: 196 - DNA, 886 bp, Zea mays subsp. mays
  misc_feature (1)..(886): ceres Seq. ID no. 12346229
aaacctcctcactcctcagactcctctccgcacacaccctccctacctcgccgtcgccgtcgcatctgccatgggggcgtacaagtacgtctcggagttatggagaaggaagcaatccgacgtgatgcggttcgttcaacgcgtgcgctgttgggagtacaggcagcagccggccatcgtccgcctcacaaggcccacccgccccgacaaggcccgccgcctcggcttcaaggccaagcaggggtatgttgtttaccgcgtccgtgtgaggcgtggtggcaggaagaggcctgtgccgaagggtattgtctatggcaagcccaagcaccagggtatcacccagctcaagttccagaggaacaagagatctgttgctgaggaacgggctggacgaaagcttggtggactcagggttctcaactcctactgggtgaatgaggatagcacctacaagtactttgagatcatcctggttgatgtggcccacactgccatccgcaacgacccaagaatcaactggctatgcaatcctgtgcacaagcatcgtgagctccgtggtctcacctctgcaggcaagaagttccgtggcctgcgtggcaagggccacactcaccacaagaacaggccgtcgaggagggccacctggaagcgcaaccagaccctgtccctccgccgctaccgttgagcacacacatcttgatgctctgctaccgtggaacccgccccatgcaggctatgatagtatttggtatttttgttgcattgttaaattttgctacccgtgttccttccgagccccttgaactgtgttgtcagtgtaccgtggagcaagtgacgcgaactatatttgtggttctcagtcaatgaaaatgattgtttgtagttt

SEQ ID NO: 197 - protein, 227 aa, Zea mays subsp. mays
  peptide (1)..(227): ceres Seq. ID no. 12346230
NLLTPQTPLRTHPPYLAVAVASAMGAYKYVSELWRRKQSDVMRFVQRVRCWEYRQQPAIVRLTRPTRPDKARRLGFKAKQGYVVYRVRVRRGGRKRPVPKGIVYGKPKHQGITQLKFQRNKRSVAEERAGRKLGGLRVLNSYWVNEDSTYKYFEIILVDVAHTAIRNDPRINWLCNPVHKHRELRGLTSAGKKFRGLRGKGHTHHKNRPSRRATWKRNQTLSLRRYR

SEQ ID NO: 198 - protein, 204 aa, Zea mays subsp. mays
  peptide (1)..(204): ceres Seq. ID no. 12346231
MGAYKYVSELWRRKQSDVMRFVQRVRCWEYRQQPAIVRLTRPTRPDKARRLGFKAKQGYVVYRVRVRRGGRKRPVPKGIVYGKPKHQGITQLKFQRNKRSVAEERAGRKLGGLRVLNSYWVNEDSTYKYFEIILVDVAHTAIRNDPRINWLCNPVHKHRELRGLTSAGKKFRGLRGKGHTHHKNRPSRRATWKRNQTLSLRRYR

SEQ ID NO: 199 - protein, 186 aa, Zea mays subsp. mays
  peptide (1)..(186): ceres Seq. ID no. 12346232
MRFVQRVRCWEYRQQPAIVRLTRPTRPDKARRLGFKAKQGYVVYRVRVRRGGRKRPVPKGIVYGKPKHQGITQLKFQRNKRSVAEERAGRKLGGLRVLNSYWVNEDSTYKYFEIILVDVAHTAIRNDPRINWLCNPVHKHRELRGLTSAGKKFRGLRGKGHTHHKNRPSRRATWKRNQTLSLRRYR

SEQ ID NO: 200 - DNA, 1829 bp, Zea mays subsp. mays
  misc_feature (1)..(1829): ceres Seq. ID no. 12349430
agaccccctctcgcttcgcccctcctcatcctctctcctctctcccccgccccgttcgtctccggcgagctagggtttgcccccccgccttgccaccccacggttcccgccatccttggatctccagccatgtacggcggagacgaggtttcggcaatcgtcatagatgtgggctcttacagctgcaaggcgggctacgcaggcgacgacacccccaaggccgtcttcccatcggttgttggttcaattgaacaaactgaagacacagatgaccccaagccagagaaggaagctgactctgcatcagattctaagaacggtgccaagcccatggatgtggacaaagccaagacaaaacgcagactttatgtaggccaagagttagagttcaggagggatcacatggaggtgatttcatctatgaaagatggaacggtaactgactgggatattgttgataatatatggaatcatgctttcagacggaggttgttgatcaatcctgaagagcatcctatgctcattgctgaaccatctacaaacactgcacagcaaagagagaaagcagcggaacttatttttgagaactacaaagtgccagcgctgtttctagccaaaaatgcagtgctcacatcttttgcgtctggacgagctacatctttggtggttgacagtggtggtgggtctactgtggtttctgctgtacacgatggttttgttttacagaagtctgtgtcgacttctccagttggcggtgaatttttaactgactgtatgatgaaaagcttggagagcaagggcatcgtcattaggcccagatattcatttaagaagaaggaaatcagtcctggagaatataagattgtagatcttgatcttccaaacacaactgagagttacaggttgtactgcatgagagctattgctagtgacgtcaaggagtctgtttgcagagttccagacactgcttttgatgaagtggcatatgcaaatgttcctacaacttcatatgagcttcctgatggacaaactattgaagttggtgctgatagattcaaggttcctgatattctatttaatccatctctatcccagactattcctgggattgatggatttgcagattcaataccagtccgtggccttcaacgaatggtcattgatagcgtaaataagtgcgacgttgacatacgaaaagagctattcagtaacatcttgctctctggtgggtcatcatcaattctgcagttaaaggagcgcctggagaaagaagtattggaggagtctcctcaagctgctcgtgtcaaggttatggcaagcggaaactcagtagagagacgtttcagtgtttggattggagggagcattctagcatctcttgggtcgttccagcagatgtggttctccaaagcagagtatgaagagcatggggtttcctatattcagagaaagtgcccttgaagcagggtaaggaatatgtacaaccaatattatgctttacgagccttattgttgccaaagggttccttgtgtacaacagcctggtgccttgccatgttggccaatgaatactcgagactcttttcaagtgacgtttgttgtcttcatggctagttaattttaggtgatgtaaggtgaacgtaagaactaggtcaactaaaatatttatcattgacagtatgctgaatctgtctccccttctgtagaatgagacgggaccccatcttgttctgtaccggggctaattctttgccagcacacaaagagccaagacgattgttgtaactcgggttatttaaatcccagctgcagagcaaatacacaacttt

SEQ ID NO: 201 - protein, 486 aa, Zea mays subsp. mays
  peptide (1)..(486): ceres Seq. ID no. 12349431
RPPLASPLLILSPLSPAPFVSGELGFAPPPCHPTVPAILGSPAMYGGDEVSAIVIDVGSYSCKAGYAGDDTPKAVFPSVVGSIEQTEDTDDPKPEKEADSASDSKNGAKPMDVDKAKTKRRLYVGQELEFRRDHMEVISSMKDGTVTDWDIVDNIWNHAFRRRLLINPEEHPMLIAEPSTNTAQQREKAAELIFENYKVPALFLAKNAVLTSFASGRATSLVVDSGGGSTVVSAVHDGFVLQKSVSTSPVGGEFLTDCMMKSLESKGIVIRPRYSFKKKEISPGEYKIVDLDLPNTTESYRLYCMRAIASDVKESVCRVPDTAFDEVAYANVPTTSYELPDGQTIEVGADRFKVPDILFNPSLSQTIPGIDGFADSIPVRGLQRMVIDSVNKCDVDIRKELFSNILLSGGSSSILQLKERLEKEVLEESPQAARVKVMASGNSVERRFSVWIGGSILASLGSFQQMWFSKAEYEEHGVSYIQRKCP

SEQ ID NO: 202 - protein, 443 aa, Zea mays subsp. mays
  peptide (1)..(443): ceres Seq. ID no. 12349432
MYGGDEVSAIVIDVGSYSCKAGYAGDDTPKAVFPSVVGSIEQTEDTDDPKPEKEADSASDSKNGAKPMDVDKAKTKRRLYVGQELEFRRDHMEVISSMKDGTVTDWDIVDNIWNHAFRRRLLINPEEHPMLIAEPSTNTAQQREKAAELIFENYKVPALFLAKNAVLTSFASGRATSLVVDSGGGSTVVSAVHDGFVLQKSVSTSPVGGEFLTDCMMKSLESKGIVIRPRYSFKKKEISPGEYKIVDLDLPNTTESYRLYCMRAIASDVKESVCRVPDTAFDEVAYANVPTTSYELPDGQTIEVGADRFKVPDILFNPSLSQTIPGIDGFADSIPVRGLQRMVIDSVNKCDVDIRKELFSNILLSGGSSSILQLKERLEKEVLEESPQAARVKVMASGNSVERRFSVWIGGSILASLGSFQQMWFSKAEYEEHGVSYIQRKCP

SEQ ID NO: 203 - protein, 376 aa, Zea mays subsp. mays
  peptide (1)..(376): ceres Seq. ID no. 12349433
MDVDKAKTKRRLYVGQELEFRRDHMEVISSMKDGTVTDWDIVDNIWNHAFRRRLLINPEEHPMLIAEPSTNTAQQREKAAELIFENYKVPALFLAKNAVLTSFASGRATSLVVDSGGGSTVVSAVHDGFVLQKSVSTSPVGGEFLTDCMMKSLESKGIVIRPRYSFKKKEISPGEYKIVDLDLPNTTESYRLYCMRAIASDVKESVCRVPDTAFDEVAYANVPTTSYELPDGQTIEVGADRFKVPDILFNPSLSQTIPGIDGFADSIPVRGLQRMVIDSVNKCDVDIRKELFSNILLSGGSSSILQLKERLEKEVLEESPQAARVKVMASGNSVERRFSVWIGGSILASLGSFQQMWFSKAEYEEHGVSYIQRKCP

SEQ ID NO: 204 - DNA, 1690 bp, Zea mays subsp. mays
  misc_feature (1)..(1690): ceres Seq. ID no. 12450440
aaagccaagcggtcagcgtcagaatagcatggcggtgtccgcggccaggatggccgtcgccacggccgtctccttagccgtgcactggctcctccggtccttcctccaggcgcagcaccccgcgctgggcctgctcttcccgaccgccgtcttcatcggcattgccgcgtctggcaacgtcgcaggcgctgccaacgacgcgccgccggggccgccggccgtgccggtgttcggcaactggctgcaggtgggcaacgacctgaaccaccgcttcctggcgcgcctgtccgcgcggtacgggcccgtcttccgcctccgcctcggcgtgcgcaacctggtggtcgtctccgacccgcgcctcgccacggaggtgctgcacacgcagggggtggagttcggctcccgcccccgcaacgtcgtattcgacatcttcaccgccaacggcgccgacatggtgttcaccgagtacggcgaccactggcgccgcatgcgccgcgtcatgacgctgcccttcttcaccgcgcgcgtcgtgcagcagtacaggggcatgtgggaggccgagatggacgccgccgtctccgacgtcgcctccgcggcccagcgcgccgccggcctcgtcgtgcggcgcaggctgcagctcatgctctacaacatcatgtacgggatgatgttcgacgcgcgcttcggctccgtcgacgaccccatgttcgtggaggccaccaggttcaactccgagcgcagccgcctcgcgcagagcttcgactacaactacggcgacttcatccccgtcctgcgcccattcctgcgcggctacctcgccagatgcaaggacctgcaggccaggcggctcgccttcttcaacagcaactacgtcgagaagaggaggaaggtcatggacgcgccgggggacaagggcaagctcaggtgcgccatcgaccacgtcctccaggcggagaagagcggcgagatcaccccggagaacgtcatctacatcgtcgagaacatcaacgtcgccgccatcgagaccacgctctggtccatcgagtgggcgctcgcggaggtcgtcaaccacccggcggtgcagcgcaaggtccgcgacgagatcaaggccgtcgtcgccgaccacgagcccatcacggagtccaccatccaccgcctcccctacctgcaggccgtcatcaaggagacgctgcgcctccactccccgatcccgctcctcgtcccgcacatgaacctcgaggaggccaagctcggcggctacaccatccccaagggctccaaggtggtggtcaacgcgtggtggctggccaacaatccggagctgtgggacaagcccgaggagtttcggccggagcggttcctaggcgaggagaagagcgtggacgccaccgtcggcgggaaggtggacttccgcttcctgccgttcggcgtgggccgccgcagctgccccgggatcatcctcgcgctgcccatcctcgcgctcatcgtcggcaagctcgtgcgcagcttcgagatggtaccaccgcccggcgtggagaagctcgacgtcagcgagaaaggaggacagttcagcctgcacattgccaaccactccgtcatcgccttccaccccgtctctgcataatagattgattgatagccaaccacgaatgctgcgcgtcgttttctgcatatcaatgttatcctaccc

SEQ ID NO: 205 - protein, 540 aa, Zea mays subsp. mays
  peptide (1)..(540): ceres Seq. ID no. 12450441
KPSGQRQNSMAVSAARMAVATAVSLAVHWLLRSFLQAQHPALGLLFPTAVFIGIAASGNVAGAANDAPPGPPAVPVFGNWLQVGNDLNHRFLARLSARYGPVFRLRLGVRNLVVVSDPRLATEVLHTQGVEFGSRPRNVVFDIFTANGADMVFTEYGDHWRRMRRVMTLPFFTARVVQQYRGMWEAEMDAAVSDVASAAQRAAGLVVRRRLQLMLYNIMYGMMFDARFGSVDDPMFVEATRFNSERSRLAQSFDYNYGDFIPVLRPFLRGYLARCKDLQARRLAFFNSNYVEKRRKVMDAPGDKGKLRCAIDHVLQAEKSGEITPENVIYIVENINVAAIETTLWSIEWALAEVVNHPAVQRKVRDEIKAVVADHEPITESTIHRLPYLQAVIKETLRLHSPIPLLVPHMNLEEAKLGGYTIPKGSKVVVNAWWLANNPELWDKPEEFRPERFLGEEKSVDATVGGKVDFRFLPFGVGRRSCPGIILALPILALIVGKLVRSFEMVPPPGVEKLDVSEKGGQFSLHIANHSVIAFHPVSA

SEQ ID NO: 206 - protein, 531 aa, Zea mays subsp. mays
  peptide (1)..(531): ceres Seq. ID no. 12450442
MAVSAARMAVATAVSLAVHWLLRSFLQAQHPALGLLFPTAVFIGIAASGNVAGAANDAPPGPPAVPVFGNWLQVGNDLNHRFLARLSARYGPVFRLRLGVRNLVVVSDPRLATEVLHTQGVEFGSRPRNVVFDIFTANGADMVFTEYGDHWRRMRRVMTLPFFTARVVQQYRGMWEAEMDAAVSDVASAAQRAAGLVVRRRLQLMLYNIMYGMMFDARFGSVDDPMFVEATRFNSERSRLAQSFDYNYGDFIPVLRPFLRGYLARCKDLQARRLAFFNSNYVEKRRKVMDAPGDKGKLRCAIDHVLQAEKSGEITPENVIYIVENINVAAIETTLWSIEWALAEVVNHPAVQRKVRDEIKAVVADHEPITESTIHRLPYLQAVIKETLRLHSPIPLLVPHMNLEEAKLGGYTIPKGSKVVVNAWWLANNPELWDKPEEFRPERFLGEEKSVDATVGGKVDFRFLPFGVGRRSCPGIILALPILALIVGKLVRSFEMVPPPGVEKLDVSEKGGQFSLHIANHSVIAFHPVSA

SEQ ID NO: 207 - protein, 524 aa, Zea mays subsp. mays
  peptide (1)..(524): ceres Seq. ID no. 12450443
MAVATAVSLAVHWLLRSFLQAQHPALGLLFPTAVFIGIAASGNVAGAANDAPPGPPAVPVFGNWLQVGNDLNHRFLARLSARYGPVFRLRLGVRNLVVVSDPRLATEVLHTQGVEFGSRPRNVVFDIFTANGADMVFTEYGDHWRRMRRVMTLPFFTARVVQQYRGMWEAEMDAAVSDVASAAQRAAGLVVRRRLQLMLYNIMYGMMFDARFGSVDDPMFVEATRFNSERSRLAQSFDYNYGDFIPVLRPFLRGYLARCKDLQARRLAFFNSNYVEKRRKVMDAPGDKGKLRCAIDHVLQAEKSGEITPENVIYIVENINVAAIETTLWSIEWALAEVVNHPAVQRKVRDEIKAVVADHEPITESTIHRLPYLQAVIKETLRLHSPIPLLVPHMNLEEAKLGGYTIPKGSKVVVNAWWLANNPELWDKPEEFRPERFLGEEKSVDATVGGKVDFRFLPFGVGRRSCPGIILALPILALIVGKLVRSFEMVPPPGVEKLDVSEKGGQFSLHIANHSVIAFHPVSA

SEQ ID NO: 208 - DNA, 457 bp, Zea mays subsp. mays
  misc_feature (1)..(457): ceres Seq. ID no. 1681038
atatagttcctgctaactgatcgagatcgctctcgtttgtactttctccctttgaggaaatagttccccttttatgtgcttcaagttcttctggtatcgtcgccagcatgggtagaggaaggattgagatcaagaggatcgagaacaacacgagccggcaggtcaccttctgcaagcgccgcaatgggctcctcaagaaggcgtacgagctctccgtcctctgcgacgctgaggtggccctcatcgtcttctctagccgtggtcgcctctacgagtatgccaacaacagtgtcaaggctactgttgagaggtacaagaaggcacacaccgttggctcttcctctgggcccccgctcctagagcacaatgcccagcaattctaccagcaagaatcagcaaaactgcgcaaccagatccagatgctgcaaaacactaacaggcacttggttggtgactc

SEQ ID NO: 209 - protein, 116 aa, Zea mays subsp. mays
  peptide (1)..(116): ceres Seq. ID no. 1681039
MGRGRIEIKRIENNTSRQVTFCKRRNGLLKKAYELSVLCDAEVALIVFSSRGRLYEYANNSVKATVERYKKAHTVGSSSGPPLLEHNAQQFYQQESAKLRNQIQMLQNTNRHLVGD

SEQ ID NO: 210 - protein, 58 aa, Zea mays subsp. mays
  peptide (1)..(58): ceres Seq. ID no. 1681040
MGSSRRRTSSPSSATLRWPSSSSLAVVASTSMPTTVSRLLLRGTRRHTPLALPLGPRS

SEQ ID NO: 211 - DNA, 811 bp, Zea mays subsp. mays
  misc_feature (1)..(811): ceres Seq. ID no. 12556477
tcttaaagcttctcgttttctctgccgtctctctcattcgcgcgacgcaaacgatcttcagccatggccaccgccgcagatgttgacgctgagattcagcaggcgctcactaacgaagtcaagctcttcaaccgctggacctatgacgacgttacggtcacagacatcagtcttgttgactacattggagttcaggcagctaaacatgctacctttgttccccacaccgctggaagatactctgtgaagagattcaggaaggctcagtgccccattgttgagaggctcaccaactctctcatgatgcacgggaggaacaacggtaagaaattgatggctgtcaggatcgtcaagcacgccatggagattatccacctcttgtctgacttgaacccaattcaggtcatcattgacgccattgtcaacagtggtccacgtgaagatgctaccagaattggatctgctggtgttgttaggagacaagctgttgatatctctcctctaagacgtgttaaccaggctatcttcttgattaccactggtgctcgtgaagctgctttcagaaacatcaagactatagctgagtgccttgctgatgaattgatcaacgcacccaagggctctttcaaacagctatgccttcaagaaagaaggatgagattgaaagagttgccaaggctaatcgttaaggattttccttttgccctgtgccgttacaatctcgtatcaatgagtttaatgttttatcttcatttagattgaaatatgtatctcagatgtttggtcttttgttttatgaagtttatttcgt

SEQ ID NO: 212 - protein, 232 aa, Zea mays subsp. mays
  peptide (1)..(232): ceres Seq. ID no. 12556478
MATAADVDAEIQQALTNEVKLFNRWTYDDVTVTDISLVDYIGVQAAKHATFVPHTAGRYSVKRFRKAQCPIVERLTNSLMMHGRNNGKKLMAVRIVKHAMEIIHLLSDLNPIQVIIDAIVNSGPREDATRIGSAGVVRRQAVDISPLRRVNQAIFLITTGAREAAFRNIKTIAECLADELINAPKGSFKQLCLQERRMRLKELPRLIVKDFPFALCRYNLVSMSLMFYLHLD

SEQ ID NO: 213 - protein, 153 aa, Zea mays subsp. mays
  peptide (1)..(153): ceres Seq. ID no. 12556479
MMHGRNNGKKLMAVRIVKHAMEIIHLLSDLNPIQVIIDAIVNSGPREDATRIGSAGVVRRQAVDISPLRRVNQAIFLITTGAREAAFRNIKTIAECLADELINAPKGSFKQLCLQERRMRLKELPRLIVKDFPFALCRYNLVSMSLMFYLHLD

SEQ ID NO: 214 - protein, 152 aa, Zea mays subsp. mays
  peptide (1)..(152): ceres Seq. ID no. 12556480
MHGRNNGKKLMAVRIVKHAMEIIHLLSDLNPIQVIIDAIVNSGPREDATRIGSAGVVRRQAVDISPLRRVNQAIFLITTGAREAAFRNIKTIAECLADELINAPKGSFKQLCLQERRMRLKELPRLIVKDFPFALCRYNLVSMSLMFYLHLD

SEQ ID NO: 215 - DNA, 435 bp, Zea mays subsp. mays
  misc_feature (1)..(435): ceres Seq. ID no. 13571374
acaactcagttattctacagctttaattttgcagagcgaccaccagtccaggtccagcgcgggacgcatcacatacacgcggtaccttcgttcgttctcaagcttatagcgcccgatcgaccctgcgcggagctagttcgttcgttccggcaggcggcggcagcgaagcagtgcgacgacgacgacgaggtacgtagagagcgagaggatagatgtcgagccgaaggtcgtcgtcgcacggcaacatctccgaggacgagatgaacgagctcgtctccaagctccaggccctgctccccagctcccgccgccgccgcggctccggccaggcgtcgacggcgaagctgctgaaggagacctgcagctacatcaagagcctccagcgggaggtggacgacctcagcgaccggctgtcggacctcttgtccaccatgg

SEQ ID NO: 216 - protein, 87 aa, Zea mays subsp. mays
  peptide (1)..(87): ceres Seq. ID no. 13571375
TTQLFYSFNFAERPPVQVQRGTHHIHAVPSFVLKLIAPDRPCAELVRSFRQAAAAKQCDDDDEVRREREDRCRAEGRRRTATSPRTR

SEQ ID NO: 217 - protein, 74 aa, Zea mays subsp. mays
  peptide (1)..(74): ceres Seq. ID no. 13571376
MSSRRSSSHGNISEDEMNELVSKLQALLPSSRRRRGSGQASTAKLLKETCSYIKSLQREVDDLSDRLSDLLSTM

SEQ ID NO: 218 - protein, 58 aa, Zea mays subsp. mays
  peptide (1)..(58): ceres Seq. ID no. 13571377
MNELVSKLQALLPSSRRRRGSGQASTAKLLKETCSYIKSLQREVDDLSDRLSDLLSTM

SEQ ID NO: 219 - DNA, 453 bp, Zea mays subsp. mays
  misc_feature (1)..(453): ceres Seq. ID no. 4616604
atcagtagtagtggcattttgcccctgctgtgccatctctctctcgctctcgctcctctcctctttttctctcctgctcagccttgcttcccgtcccatcctttctttcccttgggcgcattcagcagcttcagcctggccggccggcctgctattaaaccactttgcttgcaagtccaagaaactgctagagaggaacaagctgagatctccagcatggggaggggaaggattgagatcaagaggatcgagaacaacacgagccggcaggtcaccttctgcaagcgccgcaatgggctcctcaagaaggcgtacgagctctccgtcctctgcgacgccgaggtggcgctcgtcgtcttctccagccgcggacgcctctacgagtacgccaacaacagtgtgaaggctacgatcgagaggtacaagaaggcacacgccgttggctcttcctct

SEQ ID NO: 220 - protein, 151 aa, Zea mays subsp. mays
  peptide (1)..(151): ceres Seq. ID no. 4616605
ISSSGILPLLCHLSLALAPLLFFSPAQPCFPSHPFFPLGAFSSFSLAGRPAIKPLCLQVQETAREEQAEISSMGRGRIEIKRIENNTSRQVTFCKRRNGLLKKAYELSVLCDAEVALVVFSSRGRLYEYANNSVKATIERYKKAHAVGSSS

SEQ ID NO: 221 - protein, 133 aa, Zea mays subsp. mays
  peptide (1)..(133): ceres Seq. ID no. 4616606
SVVVAFCPCCAISLSLSLLSSFSLLLSLASRPILSFPWAHSAASAWPAGLLLNHFACKSKKLLERNKLRSPAWGGEGLRSRGSRTTRAGRSPSASAAMGSSRRRTSSPSSATPRWRSSSSPAADASTSTPTTV

SEQ ID NO: 222 - protein, 79 aa, Zea mays subsp. mays
  peptide (1)..(79): ceres Seq. ID no. 4616607
MGRGRIEIKRIENNTSRQVTFCKRRNGLLKKAYELSVLCDAEVALVVFSSRGRLYEYANNSVKATIERYKKAHAVGSSS

SEQ ID NO: 223 - DNA, 475 bp, Zea mays subsp. mays
  misc_feature (1)..(475): ceres Seq. ID no. 13633589
aaaaaaaaaaagatgcggccgttagacgagaacgagacgacggtggtcttcgagaagatcttcaaattcgtcggaaacaacctcaagaacatcgtcgagaacccatcccacgaaggacccggacccaaacccgaagccgggcgctactgcttccgcctccagaagagcagagtctactacgtaagcgagtctctcgtgaagcgagccaccaacatctcccgcaaaaacctagtctccctcggaacctgcatcgggaagtacacccacgcggggagcttccaccttacgatcatgtcgctcaacctcttggccgcgaacgcgaagcacaaggtttggcttaaacccacttcggagatgtcgtttctttacgggaaccatgtcttgaaaggagggctagggaggattaccgatagtattgtgcctggagatggggttgtcgtgttctccatgtctgatgttccgttggggtttggga

SEQ ID NO: 224 - protein, 158 aa, Zea mays subsp. mays
  peptide (1)..(158): ceres Seq. ID no. 13633590
KKKKMRPLDENETTVVFEKIFKFVGNNLKNIVENPSHEGPGPKPEAGRYCFRLQKSRVYYVSESLVKRATNISRKNLVSLGTCIGKYTHAGSFHLTIMSLNLLAANAKHKVWLKPTSEMSFLYGNHVLKGGLGRITDSIVPGDGVVVFSMSDVPLGFG

SEQ ID NO: 225 - protein, 154 aa, Zea mays subsp. mays
  peptide (1)..(154): ceres Seq. ID no. 13633591
MRPLDENETTVVFEKIFKFVGNNLKNIVENPSHEGPGPKPEAGRYCFRLQKSRVYYVSESLVKRATNISRKNLVSLGTCIGKYTHAGSFHLTIMSLNLLAANAKHKVWLKPTSEMSFLYGNHVLKGGLGRITDSIVPGDGVVVFSMSDVPLGFG

SEQ ID NO: 226 - protein, 112 aa, Zea mays subsp. mays
  peptide (1)..(112): ceres Seq. ID no. 13633592
KKKDAAVRRERDDGGLREDLQIRRKQPQEHRREPIPRRTRTQTRSRALLLPPPEEQSLLRKRVSREASHQHLPQKPSLPRNLHREVHPRGELPPYDHVAQPLGREREAQGLA

SEQ ID NO: 227 - DNA, 19 bp, Artificial Sequence
  Synthetic oligonucleotide primer
ttttttttttttttttttv

(19) United States
(12) Patent Application Publication
(10) Pub. No.: US 2007/0101460 A1
(43) Pub. Date: May 3, 2007

(54) Nucleotide sequences and polypeptides encoded thereby useful for producing transformed plants expressing lethal/nonviability genes

(75) Inventors: Kenneth Feldman, Newberry Park, CA (US); Gregory Nadzan, Woodland Hills, CA (US); Hongyu Zhang, Thousand Oaks, CA (US); Nickolai Alexandrov, Thousand Oaks, CA (US)

Correspondence Address: BIRCH STEWART KOLASCH & BIRCH, PO BOX 747, FALLS CHURCH, VA, 22040-0747, US

(73) Assignee: CERES, INC., Thousand Oaks, CA (US)

(21) Appl. No.: 10/572,221

(22) PCT Filed: Sep. 17, 2003
(86) PCT No.: PCT/US03/29054

§ 371 Date: Jan. 8, 2007

(51) Int. Cl. A01H 1/00; C07H 21/04; C12P 21/06; C07K 14/415; C12N 5/04

(52) U.S. Cl. 800/287; 435/69.1; 435/419; 435/468; 530/370; 536/23.6

(57) ABSTRACT

Isolated polynucleotides and polypeptides encoded thereby are described, together with the use of those products for making transgenic plants that are characterized by producing seeds that are not viable, not fertile, are not capable of germinating, or are otherwise not capable of regenerating into mature plants.

### FIELD OF TEE INVENTION

The present invention relates to isolated polynucleotides, polypeptides encoded thereby, and the use of those products for making transgenic plants that are characterized by expressing lethal/non-viability genes, that is, genes that when expressed/over-expressed, result in seed material that is not capable of regenerating into a mature plant.

### BACKGROUND OF THE INVENTION

There are more than 300,000 species of plants. They show a wide diversity of forms, ranging from delicate liverworts, adapted for life in a damp habitat, to cacti, capable of surviving in the desert. The plant kingdom includes herbaceous plants, such as corn, whose life cycle is measured in months, to the giant redwood tree, which m can live for thousands of years. This diversity reflects the adaptations of plants to survive in a wide range of habitats. This is seen most clearly in the flowering plants (phylum Angiospermophyta), which are the most numerous, with over 250,000 species. They are also the most widespread, being found from the tropics to the arctic.

The process of plant breeding involving man's intervention in natural breeding and selection is some 20,000 years old. It has produced remarkable advances in adapting existing species to serve new purposes. The world's economics was largely based on the successes of agriculture for most of these 20,000 years.

Plant breeding involves choosing parents, making crosses to allow recombination of gene (alleles) and searching for and selecting improved forms. Success depends on the genes/alleles available, the combinations required and the ability to create and find the correct combinations necessary to give the desired properties to the plant. Molecular genetics technologies are now capable of providing new genes, new alleles and the means of creating and selecting plants with the new, desired characteristics.

Great agronomic value can result from modulating the size of a plant as a whole or of any of its organs. For example, the green revolution came about as a result of creating dwarf wheat plants, which produced a higher seed yield than taller plants because they could withstand higher levels and inputs of fertilizer and water. Modulation of the size and stature of an entire plant or a particular portion of a plant allows productions of plants specifically improved for agriculture, horticulture and other industries. For example, reductions in height of specific ornamentals, crops and tree species can be beneficial, while increasing height of others may be beneficial.

Increasing the length of the floral stems of cut flowers in some species would also be useful, while increasing leaf size in others would be economically attractive. Enhancing the size of specific plant parts, such as seeds and fruit, to enhance yields by specifically stimulating hormone (e.g. Brassinolide) synthesis in these cells is beneficial. Another application is to stimulate early flowering by altering levels of gibberellic acid in specific cells. Changes in organ size and biomass also results in changes in the mass of constituent molecules.

It can also be of importance to produce transformed plants which in all respects are morphologically and developmentally normal, except that the seeds from those transformed plants will not germinate or otherwise produce seedlings that will mature into developed plants. For example, it may be desired to produce a transformed plant that selectively or inducibly expresses a first gene, but does not propegate to further generations.

To summarize, molecular genetic technologies provide the ability to modulate and manipulate plant size and stature of the entire plant as well as at the cell, tissue and organ levels. Thus, plant morphology can be altered to maximize the desired plant trait.

### SUMMARY OF THE INVENTION

The present invention, therefore, relates to isolated polynucleotides, polypeptides encoded thereby, and the use of those products for making transgenic plants that are characterized by being morphologically and developmentally normal, except that the seeds from the transformed plants are not capable of regenerating into a mature plant.

The present invention also relates to processes for using the isolated nucleic acid molecules and polypeptides to produce transformed plants that express, for example selectively or iducibly, a desired first gene to produce a desired trait or product and, at the same time, express a second gene that causes the plant to produce seeds that are developmentally and morphologically normal but are not capable of regenerating into a mature plant

### BRIEF DESCRIPTION OF THE INDIVIDUAL TABLES

### Table—Reference Tables

The sequences of the instant invention are described in the Sequence Listing and the Reference Table (sometimes referred to as the REF Table. The Reference Table refers to a number of “Maximum Length Sequences” or “MLS.” Each MLS corresponds to the longest cDNA and is described in the Av subsection of the Reference Table.

The Reference Table includes the following information relating to each MLS:
I. cDNA Sequence
   A. 5′ UTR
   B. Coding Sequence
   C. 3′ UTR
II. Genomic Sequence
   A. Exons
   B. Introns
   C. Promoters
III. Link of cDNA Sequences to Clone IDs
IV. Multiple Transcription Start Sites
V. Polypeptide Sequences
   A. Signal Peptide
   B. Domains
   C. Related Polypeptides
VI. Related Polynucleotide Sequences
   I. cDNA Sequence

The Reference Table indicates which sequence in the Sequence Table represents the sequence of each MLS. The MLS sequence can comprise 5′ and 3′ UTR as well as coding sequences. In addition, specific cDNA clone numbers also are included in the Reference Table when the MLS sequence relates to a specific cDNA clone.

A. 5′ UTR

The location of the 5′ UTR can be determined by comparing the most 5′ MLS sequence with the corresponding genomic sequence as indicated in the Reference Table. The sequence that matches, beginning at any of the transcriptional start sites and ending at the last nucleotide before any of the translational start sites corresponds to the 5′ UTR.

B. Coding Region

The coding region is the sequence in any open reading frame found in the MLS. Coding regions of interest are indicated in the PolyP SEQ subsection of the Reference Table.

C. 3′ UTR

The location of the 3′ UTR can be determined by comparing the most 3′ MLS sequence with the corresponding genomic sequence as indicated in the Reference Table. The sequence that matches, beginning at the translational stop site and ending at the last nucleotide of the MLS corresponds to the 3′ UTR.

II. Genomic Sequence

Further, the Reference Table indicates the specific “gi” number of the genomic sequence if the sequence resides in a public databank. For each genomic sequence, Reference tables indicate which regions are included in the MLS. These regions can include the 5′ and 3′ UTRs as well as the coding sequence of the MLS. See, for example, the scheme below:

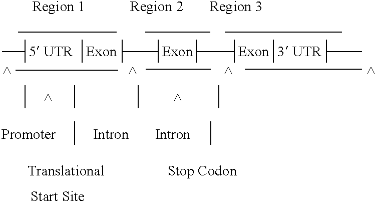

The Reference Table reports the first and last base of each region that are included in an MLS sequence. An example is shown below:

gi No. 47000:

37102 . . . 37497

37593 . . . 37925

The numbers indicate that the MLS contains the following sequences from two regions of gi No. 47000; a first region including bases 37102-37497, and a second region including bases 37593-37925.

A. Exon Sequences

The location of the exons can be determined by comparing the sequence of the regions from the genomic sequences with the corresponding MLS sequence as indicated by the Reference Table.

i. Initial Exon

To determine the location of the initial exon, information from the
(1) polypeptide sequence section;
(2) cDNA polynucleotide section; and
(3) the genomic sequence section
   of the Reference Table is used. First, the polypeptide section will indicate where the translational start site is located in the MLS sequence. The MLS sequence can be matched to the genomic sequence that corresponds to the MLS. Based on the match between the MLS and corresponding genomic sequences, the location of the translational start site can be determined in one of the regions of the genomic sequence. The location of this translational start site is the start of the first exon.

Generally, the last base of the exon of the corresponding genomic region, in which the translational start site was located, will represent the end of the initial exon. In some cases, the initial exon will end with a stop codon, when the initial exon is the only exon.

In the case when sequences representing the MLS are in the positive strand of the corresponding genomic sequence, the last base will be a larger number than the first base. When the sequences representing the MLS are in the negative strand of the corresponding genomic sequence, then the last base will be a smaller number than the first base.

ii. Internal Exons

Except for the regions that comprise the 5′ and 3′ UTRs, initial exon, and terminal exon, the remaining genomic regions that match the MLS sequence are the internal exons. Specifically, the bases defining the boundaries of the remaining regions also define the intron/exon junctions of the internal exons.

iii. Terminal Exon

As with the initial exon, the location of the terminal exon is determined with information from the
(1) polypeptide sequence section;
(2) cDNA polynucleotide section; and
(3) the genomic sequence section
   of the Reference Table. The polypeptide section will indicate where the stop codon is located in the MLS sequence. The MLS sequence can be matched to the corresponding genomic sequence. Based on the match between MLS and corresponding genomic sequences, the location of the stop codon can be determined in one of the regions of the genomic sequence. The location of this stop codon is the end of the terminal exon. Generally, the first base of the exon of the corresponding genomic region that matches the cDNA sequence, in which the stop codon was located, will represent the beginning of the terminal exon. In some cases, the translational start site will represent the start of the terminal exon, which will be the only exon.

In the case when the MLS sequences are in the positive strand of the corresponding genomic sequence, the last base will be a larger number than the first base. When the MLS sequences are in the negative strand of the corresponding genomic sequence, then the last base will be a smaller number than the first base.

B. Intron Sequences

In addition, the introns corresponding to the MLS are defined by identifying the genomic sequence located between the regions where the genomic sequence comprises exons. Thus, introns are defined as starting one base downstream of a genomic region comprising an exon, and end one base upstream from a genomic region comprising an exon.

C. Promoter Sequences

As indicated below, promoter sequences corresponding to the MLS are defined as sequences upstream of the first exon; more usually, as sequences upstream of the first of multiple transcription start sites; even more usually as sequences about 2,000 nucleotides upstream of the first of multiple transcription start sites.

III. Link of cDNA Sequences to Clone IDs

As noted above, the Reference Table identifies the cDNA clone(s) that relate to each MLS. The MLS sequence can be longer than the sequences included in the cDNA clones. In such a case, the Reference Table indicates the region of the MLS that is included in the clone. If either the 5′ or 3′ termini of the cDNA clone sequence is the same as the MLS sequence, no mention will be made.

IV. Multiple Transcription Start Sites

Initiation of transcription can occur at a number of sites of the gene. The Reference Table indicates the possible multiple transcription sites for each gene. In the Reference Table, the location of the transcription start sites can be either a positive or negative number.

The positions indicated by positive numbers refer to the transcription start sites as located in the MLS sequence. The negative numbers indicate the transcription start site within the genomic sequence that corresponds to the MLS.

To determine the location of the transcription start sites with the negative numbers, the MLS sequence is aligned with the corresponding genomic sequence. In the instances when a public genomic sequence is referenced, the relevant corresponding genomic sequence can be found by direct reference to the nucleotide sequence indicated by the “gi” number shown in the public genomic DNA section of the Reference Table. When the position is a negative number, the transcription start site is located in the corresponding genomic sequence upstream of the base that matches the beginning of the MLS sequence in the alignment. The negative number is relative to the first base of the MLS sequence which matches the genomic sequence corresponding to the relevant “gi” number.

In the instances when no public genomic DNA is referenced, the relevant nucleotide sequence for alignment is the nucleotide sequence associated with the amino acid sequence designated by “gi” number of the later PolyP SEQ subsection.

V. Polypeptide Sequences

The PolyP SEQ subsection lists SEQ ID NOS. and Ceres SEQ ID NO for polypeptide sequences corresponding to the coding sequence of the MLS sequence and the location of the translational start site with the coding sequence of the MLS sequence.

The MLS sequence can have multiple translational start sites and can be capable of producing more than one polypeptide sequence.

Subsection (Dp) provides (where present) information concerning amino acid sequences that are found to be related and have some percentage of sequence identity to the polypeptide sequences of the Reference and Sequence Tables. These related sequences are identified by a “gi” number.

### DETAILED DESCRIPTION OF THE INVENTION

### 1. Definitions

The following terms are utilized throughout this application:
Allelic variant: An “allelic variant” is an alternative form of the same SDF, which resides at the same chromosomal locus in the organism. Allelic variations can occur in any portion of the gene sequence, including regulatory regions. Allelic variants can arise by normal genetic variation in a population. Allelic variants can also be produced by genetic engineering methods. An allelic variant can be one that is found in a naturally occurring plant, including a cultivar or ecotype. An allelic variant may or may not give rise to a phenotypic change, and may or may not be expressed. An allele can result in a detectable change in the phenotype of the trait represented by the locus. A phenotypically silent allele can give rise to a product.
Chimeric: The term “chimeric” is used to describe genes, as defined supra, or contracts wherein at least two of the elements of the gene or construct, such as the promoter and the coding sequence and/or other regulatory sequences and/or filler sequences and/or complements thereof, are heterologous to each other.
Constitutive Promoter: Promoters referred to herein as “constitutive promoters” actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.
Coordinately Expressed: The term “coordinately expressed,” as used in the current invention, refers to genes that are expressed at the same or a similar time and/or stage and/or under the same or similar environmental conditions.
Domain: Domains are fingerprints or signatures that can be used to characterize protein families and/or parts of proteins. Such fingerprints or signatures can comprise conserved (1) primary sequence, (2) secondary structure, and/or (3) three dimensional conformation. Generally, each domain has been associated with either a family of proteins or motifs. Typically, these families and/or motifs have been correlated with specific in-vitro and/or in-vivo activities. A domain can be any length, including the entirety of the sequence of a protein. Detailed descriptions of the domains, associated families and motifs, and correlated activities of the polypeptides of the instant invention are described below. Usually, the polypeptides with designated domain(s) can exhibit at least one activity that is exhibited by any polypeptide that comprises the same domain(s).
Endogenous: The term “endogenous,” within the context of the current invention refers to any polynucleotide, polypeptide or protein sequence which is a natural part of a cell or organisms regenerated from said cell.
Exogenous: “Exogenous,” as referred to within, is any polynucleotide, polypeptide or protein sequence, whether chimeric or not, that is initially or subsequently introduced into the genome of an individual host cell or the organism regenerated from said host cell by any means other than by a sexual cross. Examples of means by which this can be accomplished are descried below, and include Agrobacterium-mediated transformation (of dicots—e.g. Salomon et al. EMBO J. 3:141 (1984); Herrera-Estrella et al. EMBO J. 2:987 (1983); of monocots, representative papers are those by Escudero et al., Plant J. 10:355 (1996), Ishida et al., Nature Biotechnology 14:745 (1996), May et al., Bio/Technology 13:486 (1995)), biolistic methods (Armaleo et al., Current Genetics 17:97 1990)), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.
Gene: The term “gene,” as used in the context of the current invention, encompasses all regulatory and coding sequence contiguously associated with a single hereditary unit with a genetic function. Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes comprised of “exons” (coding sequences), which may be interrupted by “introns” (non-coding sequences), encode proteins. A gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, artificial chromosome, plasmid, vector, etc., or as a separate isolated entity.
Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence originates from, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.
Homologous gene: In the current invention, “homologous gene” refers to a gene that shares sequence similarity with the gene of interest. This similarity may be in only a fragment of the sequence and often represents a functional domain such as, examples including without limitation a DNA binding domain, a domain with tyrosine kinase activity, or the like. The functional activities of homologous genes are not necessarily the same.
Inducible Promoter: An “inducible promoter” in the context of the current invention refers to a promoter which is regulated under certain conditions, such as light, chemical concentration, protein concentration, conditions in an organism cell, or organelle, etc. A typical example of an inducible promoter, which can be utilized with the polynucleotides of the present invention, is PARSK1, the promoter from the Arabidopsis gene encoding a serine-threonine kinase enzyme, and which promoter is induced by dehydration, abscissic acid and sodium chloride (Wang and Goodman, Plant J. 8:37 (1995)). Examples of environmental conditions that may affect transcription by inducible promoters include anaerobic conditions, elevated temperature, or the presence of light.
Orthologous Gene: In the current invention “orthologous gene” refers to a second gene that encodes a gene product that performs a similar function as the product of a first gene. The orthologous gene may also have a degree of sequence similarity to the first gene. The orthologous gene may encode a polypeptide that exhibits a degree of sequence similarity to a polypeptide corresponding to a first gene. The sequence similarity can be found within a functional domain or along the entire length of the coding sequence of the genes and/or their corresponding polypeptides.
Percentage of sequence identity: “Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math 2:482 (1981), by the homology alignment algorithm of Needleman and Wunsch. J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 80% sequence identity, preferably at lest 85%, more preferably at least 90% and most preferably at least 95%, even more preferably, at least 96%, 97%, 98% or 99% sequence identity compared to a reference sequence using the programs.
Plant Promoter: A “plant promoter” is a promoter capable of initiating transcription in plant cells and can drive or facilitate transcription of a fragment of the SDF of the instant invention or a coding sequence of the SDF of the instant invention. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-1 (ubi-1)promoter known to those of skill.
Promoter: The term “promoter,” as used herein, refers to a region of sequence determinants located upstream from the start of transcription of a gene and which are involved in recognition and binding of RNA polymerase and other proteins to initiate and modulate transcription. A basal promoter is the minimal sequence necessary for assembly of a transcription complex required for transcription initiation. Basal promoters frequently include a “TATA box” element usually located between 15 and 35 nucleotides upstream from the site of initiation of transcription. Basal promoters also sometimes include a “CCAAT box” element (typically a sequence CCAAT) and/or a GGGCG sequence, usually located between 40 and 200 nucleotides, preferably 60 to 120 nucleotides, upstream from the start site of transcription.
Regulatory Sequence: The term “regulatory sequence,” as used in the current invention, refers to any nucleotide sequence that influences transcription or translation initiation and rate, and stability and/or mobility of the transcript or polypeptide product. Regulatory sequences include, but are not limited to, promoters, promoter control elements, protein binding sequences, 5′ and 3′ UTRs, transcriptional start site, termination sequence, polyadenylation sequence, introns, certain sequences within a coding sequence, etc.
Signal Peptide: A “signal peptide” as used in the current invention is an amino acid sequence that targets the protein for secretion, for transport to an intracellular compartment or organelle or for incorporation into a membrane. Signal peptides are indicated in the tables and a more detailed description located below.
Specific Promoter: In the context of the current invention, “specific promoters” refers to a subset of inducible promoters that have a high preference for being induced in a specific tissue or cell and/or at a specific time during development of an organism. By “high preference” is meant at least 3-fold, preferably 5-fold, more preferably at least 10-fold still more preferably at least 20-fold, 50-fold or 100-fold increase in transcription in the desired tissue over the transcription in any other tissue. Typical examples of temporal and/or tissue specific promoters of plant origin that can be used with the polynucleotides of the present invention, are: PTA29, a promoter which is capable of driving gene transcription specifically in tapetum and only during anther development (Koltonow et al., Plant Cell 2:1201 (1990); RCc2 and RCc3, promoters that direct root-specific gene transcription in rice (Xu et al., Plant Mol. Biol. 27:237 (1995); TobRB27, a root-specific promoter from tobacco (Yamamoto et al., Plant Cell 3:371 (1991)). Examples of tissue-specific promoters under developmental control include promoters that initiate transcription only in certain tissues or organs, such as root, ovule, fruit, seeds, or flowers. Other suitable promoters include those from genes encoding storage proteins or the lipid body membrane protein, oleosin. A few root-specific promoters are noted above.
Stringency: “Stringency” as used herein is a function of probe length, probe composition (G+C content), and salt concentration, organic solvent concentration, and temperature of hybridization or wash conditions. Stringency is typically compared by the parameter T_{m,} which is the temperature at which 50% of the complementary molecules in the hybridization are hybridized, in terms of a temperature differential from Tₘ. High stringency conditions are those providing a condition of Tₘ−5° C. to Tₘ−10° C. Medium or moderate stringency conditions are those providing Tₘ−20° C. to Tₘ−29° C. Low stringency conditions are those providing a condition of Tₘ−40° C. to Tₘ−48° C. The relationship of hybridization conditions to Tₘ (in ° C.) is expressed in the mathematical equation
   Tₘ=81.5−16.6(log₁₀[Na⁺])+0.41(% G+C)−(600/N) (1)
   where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).
   Tₘ=81.5+16.6 log {[Na⁺]/(1+0.7[Na⁺)}+0.41(% G+C)−500/L 0.63(% formamide) (2)
   where L is the length of the probe in the hybrid. (P. Tijessen, “Hybridization with, Nucleic Acid Probes” in Laboratory Techniques in Biochemistry and Molecular Biology, P. C. vand der Vliet, ed, c. 1993 by Elsevier, Amsterdam.) The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tₘ decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.
Substantially free of: A composition containing A is “substantially free of” B when at least 85% by weight of the total A+B in the composition is A. Preferably, A comprises at least about 90% by weight of the total of A+B in the composition, more preferably at least about 95% or even 99% by weight. For example, a plant gene or DNA sequence can be considered substantially free of other plant genes or DNA sequences.
Translational start site: In the context of the current invention, a “translational start site” is usually an ATG in the cDNA transcript, more usually the first ATG. A single cDNA, however, may have multiple translational start sites.
Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single gene may have multiple transcriptional start sites, some of which may be specific for transcription in a particular cell-type or tissue.
Untranslated region (UTR): A. “UTR” is any contiguous series of nucleotide bases that is transcribed, but is not translated. These untranslated regions may be associated with particular functions such as increasing mRNA message stability. Examples of UTRs include, but are not limited to polyadenylation signals, terminations sequences, sequences located between the transcriptional start site and the first exon (5′ UTR) and sequences located between the last exon and the end of the mRNA (3′ UTR).
Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc).

### 2. Important Characteristics of the Polynuceotides of the Invention

The genes and polynucleotides of the present invention are of interest because when they are over-expressed (i.e. when expressed in an increased amount) they produce plants that are in all respects morphologically, and developmentally normal, except that the seeds from those plants will not germinate or otherwise produce viable seedlings that will develop into mature plants; i.e. the seeds are non-viable.

### 3. The Genes of the Invention

The sequences of the invention were isolated from Arabidopsis and other species, and are considered orthologous genes because the polypeptides perform similar functions in a transgenic plant.

Based upon the orthologous sequences, Applicants have determined that plants having the desired characteristics discussed above can be obtained by transformation of a plant or plant cell with a polynucleotide (stably integrated into the plant genome) that codes for a polypeptide that comprises one of the consensus sequences described in Table 3.

The consensus sequence contains both lower-case and upper-case letters. The upper-case letters represent the standard one-letter amino acid abbreviations. The lower case letters represent classes of amino acids:
“t” refers to tiny amino acids, which are specifically alanine, glycine, serine and threonine.
“p” refers to polar amino acids, which are specifically, asparagine and glutamine
“n” refers to negatively charged amino acids, which are specifically, aspartic acid and glutamic acid
“+” refers to positively charged residues, which are specifically, lysine, arginine, and histidine
“r” refers to aromatic residues, which are specifically, phenylalanine, tyrosine, and tryptophan,
“a” refers to aliphatic residues, which are specifically, isoleucine, valine, leucine, and methonine
“< >” refers to the number of residues present. For example, A <8>S indicates that eight residues separate the alanine residue from the serine residue. “A<8>S” is equivalent to “A XXXXX XXXS.” Likewise “A<1-3>S” indicates that at least one, but as many as three residues separate alanine from serine.

In addition to each consensus sequence of the invention in Table 3, Applicants have generated a scoring matrix to provide further description of the consensus sequence. Table 4 describes the scoring for each consensus sequence. The first row of each matrix indicates the residue position in the consensus sequence. The matrix reports the number of occurrences of all the amino acids that were found in the group members for every residue position of the signature sequence. The matrix also indicates for each residue position, how many different organisms were found to have a polypeptide in the group that included a residue at the relevant position. The last line of the matrix indicates all the amino acids that were found at each position of the consensus.

Table 5 groups the individual sequences of the invention into groups of orthologous sequences, with each group being identified by a number in the left-most column labeled “Group”. Additional information is then provided for each sequence (set forth in a row), namely each sequence is correlated with it's ortholog group number and is identified by either a “gi number” (if it is a sequence known in the public NCBI non-redundant database) or by a Ceres “cDNA ID” and/or “Peptide ID” number, followed by an identification of the relevant plant species. Each sequence was blasted against the public databases (both the NCBI non-redundant database and the Derwent database) to determine the amount of sequence similarity with any publically available sequences. Table 5 presents the results of those blast comparisons noting the total length of the sequence of the invention being blasted against the database, the number of positional matches in the sequence alignment to a sequence in the database, and the percent sequence similarity in that alignment.

In addition to the sequences of the invention set forth in the individual tables, the invention also encompasses variants, fragments or fusions of the polypeptides that produce the same phenotypic effect after transformation into a host plant.

As noted above, the described consensus sequences show the conserved residues of homologous sequences from different species. These consensus sequences can guide those skilled in the art to construct mutants, fragments or fusions of the naturally occurring sequences, which will retain the desired function(s).

It is understood that the variation between homologous sequences is typically higher at the N-terminus and the C-terminus of the proteins. Thus, the present invention includes the fragments of the consensus sequences. Of particular interest are those that are shorter at the N-terminus than the consensus shown in the application. The consensus can be shortened at the N-terminus or C-terminus by up to 10% of the total length of the consensus or up to 10 amino acids at either end of the consensus.

A type of variant of the polypeptides comprises amino acid substitutions. Conservative substitutions are preferred to maintain the function or activity of the polypeptide. Such substitutions include conservation of charge, polarity, hydrophobicity, size, etc. For example, one or more amino acid residues within the sequence can be substituted with another amino acid of similar polarity that acts as a functional equivalent, for example providing a hydrogen bond in an enzymatic catalysis. Substitutes for an amino acid within an exemplified sequence are preferably made among the members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

The variants include those that have a percentage of sequence identity to the sequences of the invention with the range of at least 80%, or preferably at least 85, 90, 95, 96, 97, 98 or 99%. Within that scope of percentage of sequence identity, a polypeptide of the invention may have additional individual amino acids or amino acid sequences inserted into the polypeptide in the middle thereof and/or at the N-terminal and/or C-terminal ends thereof. Likewise, some of the amino acids or amino acid sequences may be deleted from the polypeptide. Amino acid substitutions may also be made in the sequences; conservative substitutions being preferred.

One preferred class of variants are those that comprise (1) the domain of an encoded polypeptide and/or (2) residues conserved between the encoded polypeptide and related polypeptides. For this class of variants, the encoded polypeptide sequence is changed by insertion, deletion, or substitution at positions flanking the domain and/or conserved residues. Another class of variants includes those that comprise an encoded polypeptide sequence that is changed in the domain or conserved residues by a conservative substitution.

### 4. Use of the Genes to Make Transgenic Plants

To use the sequences of the present invention or a combination of them or parts and/or mutants and/or fusions and/or variants of them, recombinant DNA constructs are prepared which comprise the polynucleotide sequences of the invention inserted into a vector, and which are suitable for transformation of plant cells. The construct can be made using standard recombinant DNA techniques (Sambrook et al. 1989) and can be introduced to the species of interest by Agrobacterium-mediated transformation or by other means of transformation as referenced below.

The vector backbone can be any of those typical in the art such as plasmids, viruses, artificial chromosomes, BACs, YACs and PACs and vectors of the sort described by
(a) BAC: Shizuya et al., Proc. Natl. Acad. Sci. USA 89: 8794-8797 (1992); Hamilton et al., Proc. Natl. Acad. Sci. USA 93: 9975-9979 (1996);
(b) YAC: Burke et al., Science 236:806-812 (1987);
(c) PAC: Sternberg N. et al., Proc Natl Acad Sci USA. January;87(1):103-7 (1990);
(d) Bacteria-Yeast Shuttle Vectors: Bradshaw et al., Nucl Acids Res 23: 4850-4856 (1995);
(e) Lambda Phage Vectors: Replacement Vector, e.g., Frischauf et al., J. Mol Biol 170: 827-842 (1983); or Insertion vector, e.g., Huynh et al., In: Glover N M (ed) DNA Cloning: A practical Approach, Vol. 1 Oxford: IRL Press (1985); T-DNA gene fusion vectors: Walden et al., Mol Cell Biol 1: 175-194 (1990); and
(g) Plasmid vectors: Sambrook et al., infra.

Typically, the construct will comprise a vector containing a sequence of the present invention with any desired transcriptional and/or translational regulatory sequences, such as promoters, UTRs, and 3′ end termination sequences. Vectors can also include origins of replication, scaffold attachment regions (SARs), markers, homologous sequences, introns, etc. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron or phosphinotricin.

A plant promoter fragment may be used that directs transcription of the gene in all issues of a regenerated plant and may be a constitutive promoter, such as 35S. Alternatively, the plant promoter may direct transcription of a sequence of the invention in a specific tissue (tissue-specific promoters) or may be otherwise under more precise environmental control (inducible promoters).

If proper polypeptide production is desired, a polyadenylation region at the 3′-end of the coding region is typically included. The polyadenylation region can be derived from the natural gene, from a variety of other plant genes, or from T-DNA.

Knock-In Constructs

Ectopic expression of the sequences of the invention can also be accomplished using a “knock-in” approach. Here, the first component, an “activator line,” is created by generating a transgenic plant comprising a transcriptional activator operatively linked to a promoter. The second component comprises the desired cDNA sequence operatively linked to the target binding sequence/region of the transcriptional activator. The second component can be transformed into the “activator line” or be used to transform a host plant to produce a “target” line that can be crossed with the “activator line” by ordinary breeding methods. In either case, the result is the same. That is, the promoter drives production of the transcriptional activator protein that then binds to the target binding region to facilitate expression of the desired cDNA.

Any promoter that functions in plants can be used in the first component, such as the 35S Cauliflower Mosaic Virus promoter or a tissue or organ specific promoter. Suitable transcriptional activator polypeptides include, but are not limited to, those encoding HAP1 and GAL4. The binding sequence recognized and targeted by the selected transcriptional activator protein is used in the second component.

Transformation

Techniques for transforming a wide variety of higher plant species are well known and described in the technical and scientific literature. See, e.g. Weising et al, Ann. Rev. Genet. 22:421 (1988); and Christou, Euphytica, v. 85, n.1-3:13-27, (1995).

Processes for the transformation of monocotyledonous and dicotyledonous plants are known to the person skilled in the art. For the introduction of DNA into a plant host cell a variety of techniques is available. These techniques comprise the transformation of plant cells with T-DNA using Agrobacterium tumefaciens or Agrobacterium rhizogenes as transformation means, the fusion of protoplasts, the injection, the electroporation of DNA, the introduction of DNA by means of the biolistic method as well as further possibilities.

For the injection and electroporation of DNA in plant cells the plasmids do not have to fulfill specific requirements. Simple plasmids such as pUC derivatives can be used.

The use of agrobacteria for the transformation of plant cells has extensively been examined and sufficiently disclosed in the specification of EP-A 120 516, in Hoekema (In: The Binary Plant Vector System Offsetdrukkerij Kanters B. V., Alblasserdam (1985), Chapter V), Fraley et al. (Crit. Rev. Plant. Sci. 4, 1-46) and An et al. (EMBO J. 4 (1985), 277-287).

For the transfer of the DNA to the plant cell plant explants can be co-cultivated with Agrobacterium tumefaciens or Agrobacterium rhizogenes. From the infected plant material (for example leaf explants, segments of stems, roots but also protoplasts or suspension cultivated plant cells) whole plants can be regenerated in a suitable medium which may contain antibiotics or biozides for the selection of transformed cells. The plants obtained that way can then be examined for the presence of the introduced DNA. Other possibilities for the introduction of foreign DNA using the biolistic method or by protoplast transformation are known (cf., e.g., Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

The transformation of dicotyledonous plants via Ti-plasmid-vector systems with the help of Agrobacterium tumefaciens is well-established. Recent studies have indicated that also monocotyledonous plants can be transformed by means of vectors based on Agrobacterium (Chan et al., Plant Mol. Biol. 22 (1993), 491-506; Hiei et al., Plant J. 6 (1994), 271-282; Deng et al., Science in China 33 (1990), 28-34; Wilmink et al., Plant Cell Reports 11 (1992), 76-80; May et al., Bio/Technology 13 (1995), 486-492; Conner and Domisse; Int. J. Plant Sci. 153 (1992), 550-555; Ritchie et al., Transgenic Res. 2 (1993), 252-265).

Alternative systems for the transformation of monocotyledonous plants are the transformation by means of the biolistic method (Wan and Lemaux, Plant Physiol. 104 (1994), 37-48; Vasil et al., Bio/Technology 11 (1993), 1553-1558; Ritala et al., Plant Mol. Biol. 24 (1994), 317-325; Spencer et al., Theor. Appl. Genet. 79 (1990), 625-631), the protoplast transformation, the electroporation of partially permeabilized cells, as well as the introduction of DNA by means of glass fibers.

In particular the transformation of maize is described in the literature several times (cf, e.g., WO95/06128, EP 0 513 849; EP 0 465 875; Fromm et al., Biotechnology 8 (1990), 833-844; Gordon-Kamm et al., Plant Cell 2 (1990), 603-618; Koziel et al., Biotechnology 11 (1993), 194-200). In EP 292 435 and in Shillito et al. (Bio/Technology 7 (1989), 581) a process is described with the help of which and starting from a mucus-free, soft (friable) maize callus fertile plants can be obtained. Prioli and Söndahl (Bio/Technology 7 (1989), 589) describe the regenerating and obtaining of fertile plants from maize protoplasts of the Cateto maize inbred line Cat 100-1.

The successful transformation of other cereal species has also been described, for example for barley (Wan and Lemaux, see above; Ritala et al., see above) and for wheat (Nehra et al., Plant J. 5(1994), 285-297).

Once the introduced DNA has been integrated into the genome of the plant cell, it usually is stable there and is also contained in the progenies of the originally transformed cell. It usually contains a selection marker which makes the transformed plant cells resistant to a biozide or an antibiotic such as kanamycin, G 418, bleomycin, hygromycin or phosphinotricin and others. Therefore, the individually chosen marker should allow the selection of transformed cells from cells lacking the introduced DNA.

The transformed cells grow within the plant in the usual way (see also McCormick et al., Plant Cell Reports 5 (1986), 81-84). The resulting plants can be cultured normally. Seeds can be obtained from the plants.

Two or more generations should be cultivated to make sure that the phenotypic feature is maintained stably and is transmitted. Seeds should be harvested to make sure that the corresponding phenotype or other properties are maintained.

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria (McCormac et al., Mol. Biotechnol. 8:199 (1997); Hamilton, Gene 200:107 (1997)); Salomon et al. EMBO J. 3:141 (1984); Herrera-Estrella et al. EMBO J. 2:987 (1983).

Microinjection techniques are known in the art and well described in the scientific and patent literature. The introduction of DNA constructs using polyethylene glycol precipitation is described in Paszkowski et al. EMBO J. 3:2717 (1984). Electroporation techniques are described in Fromm et al. Proc. Natl Acad. Sci. USA 82:5824 (1985). Ballistic transformation techniques are described in Klein et al. Nature 327:773 (1987). Agrobacterium tumefaciens-mediated transfomation techniques, including disarming and use of binary or co-integrate vectors, are well described in the scientific literature. See, for example Hamilton, C. M., Gene 200:107 (1997); Müller et al. Mol. Gen. Genet. 207:171 (1987); Komari et al. Plant J. 10:165 (1996); Venkateswarlu et al. Biotechnology 9:1103 (1991) and Gleave, A. P., Plant Mol. Biol. 20:1203 (1992); Graves and Goldman, Plant Mol. Biol. 7:34 (1986) and Gould et al., Plant Physiology 95:426 (1991).

Transformed plant cells that have been obtained by any of the above transformation techniques can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture in “Handbook of Plant Cell Culture,” pp. 124-176, MacMillan Publishing Company, New York, 1983; and Binding, Regeneration of Plants, Plant Protoplasts, pp. 21-73, CRC Press, Boca Raton, 1988. Regeneration can also be obtained from plant callus, explants, organs, or parts thereof. Such regeneration techniques are described generally in Klee et al. Ann. Rev. of Plant Phys. 38:467 (1987). Regeneration of monocots (rice) is described by Hosoyama et al. (Biosci. Biotechnol. Biochem. 58:1500 (1994)) and by Ghosh et al. (J. Biotechnol. 32:1 (1994)). The nucleic acids of the invention can be used to confer the trait of increased height, increased primary inflorescence thickness, an increase in the number and size of leaves and a delay in flowering time, without reduction in fertility, on essentially any plant.

The nucleotide sequences according to the invention can generally encode any appropriate proteins from any organism, in particular from plants, fungi, bacteria or animals. The sequences preferably encode proteins from plants or fungi. Preferably, the plants are higher plants, in particular starch or oil storing useful plants, for example potato or cereals such as rice, maize, wheat, barley, rye, triticale, oat, millet, etc., as well as spinach, tobacco, sugar beet, soya, cotton etc.

The process according to the invention can in principle be applied to any plant. Therefore, monocotyledonous as well as dicotyledonous plant species are particularly suitable. The process is preferably used with plants that are interesting for agriculture, horticulture and/or forestry.

Examples thereof are vegetable plants such as, for example, cucumber, melon, pumpkin, eggplant, zucchini, tomato, spinach, cabbage species, peas, beans, etc., as well as fruits such as, for example, pears, apples, etc.

Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

To prepare transformed plants that express a first desired gene (such as genes that code for a desired protein product) but which plants produce seeds that are not viable, do not germinate, result in seedlings that die quickly, or are otherwise not capable of regenerating into mature plants, it is possible to transform plants with the polynucleotides of the present invention according to the procedures and teachings described in co-pending application Ser. No. ______, entitled “Biological Containment System” and filed on Sep. 17, 2003, claiming priority on provisional application Ser. No. 60/411,823, filed on Sep. 17, 2002, the contents of both applications being hereby fully incorporated by reference.

### 5. Phenotype Studies

The genes of the invention were utilized to transform plants (specifically Arabidopsis as a model species) and the results show the improved phenotype characteristics of the transgenic plants as described above.

Materials and Methods:

Generation and Phenotypic Evaluation of Transformants

Wild-type Arabidopsis Wassilewskija (WS) plants were transformed with a Ti plasmid containing the cDNA of interest 12337825 in the sense orientation relative to a known promoter, such as a broad spectrum promoter that expresses in most cells, or constitutive promoter (such as 35S). The Ti plasmid vector used for this construct, contains a plant selectable marker gene, such as phosphinothricin acetyltransferase (PAT), that confers herbicide resistance to transformed plants. The transformation is conducted as follows:

Procedure: Agrobacterium-Mediated Transformation of Arabidopsis

Materials:

0.2% Phytagar
   2 g Phytagar
   1 L nanopure water
YEB (for 1 L)
   5 g extract of meat
   5 g Bacto peptone
   1 g yeast extract
   5 g sucrose
   0.24 g magnesium sulfate
Infiltration Medium (IM) (for 1 L)
   2.2 g MS salts
   50 g sucrose
   5 ul BAP solution (stock is 2 mg/ml)
      Methods:
1. Stratification of WS-2 Seed.
   Add 0.5 ml WS-2 (CS2360) seed to 50 ml of 0.2% Phytagar in a 50 ml Corning tube and vortex until seeds and Phytagar form a homogenous mixture.
   Cover tube with foil and stratify at 4° C. for 3 days.
2. Preparation of Seed Mixture.
   Obtain stratified seed from cooler.
   Add seed mixture to a 1000 ml beaker.
   Add an additional 950 ml of 0.2% Phytagar and mix to homogenize.
3. Preparation of Soil Mixture.
   Mix 24 L SunshineMix #5 soil with 16 L Therm-O-Rock vermiculite in cement mixer to make a 60:40 soil mixture.
   Amend soil mixture by adding 2 Tbsp Marathon and 3 Tbsp Osmocote and mix contents thoroughly.
   Add 1 Tbsp Peters fertilizer to 3 gallons of water and add to soil mixture and mix thoroughly.
   Fill 4-inch pots with soil mixture and round the surface to create a slight dome.
   Cover pots with 8-inch squares of nylon netting and fasten using rubber bands.
   Place 14 4-inch pots into each no-hole utility flat.
4. Planting.
   Using a 60 ml syringe, aspirate 35 ml of the seed mixture.
   Exude 25 drops of the seed mixture onto each pot.
   Repeat until all pots have been seeded.
   Place flats on greenhouse bench, cover flat with clear propagation domes, place 55% shade cloth on top of flats and subirrigate by adding 1 inch of water to bottom of each flat.
5. Preparation of Agrobacterium.
   Add 150 ml fresh YEB to 250 ml centrifuge bottles and cap each with a foam plug (Identi-Plug).
   Autoclave for 40 min at 121° C.
   After cooling to room temperature, uncap and add 0.1 ml each of carbenicillin, spectinomycin and rifampicin stock solutions to each culture vessel.
   Obtain Agrobacterium starter block (96-well block with Agrobacterium cultures grown to an OD₆₀₀ of approximately 1.0) and inoculate one culture vessel per construct by transferring 1 ml from appropriate well in the starter block.
   Cap culture vessels and place on Lab-Line incubator shaker set at 27° C. and 250 RPM.
   Remove after Agrobacterium cultures reach an OD₆₀₀ of approximately 1.0 (about 24 hours), cap culture vessels with plastic caps, place in Sorvall SLA 1500 rotor and centrifuge at 8000 RPM for 8 min at 4° C.
   Pour out supernatant and put bottles on ice until ready to use.
   Add 200 ml Infiltration Media (IM) to each bottle, resuspend Agrobacterium pellets and store on ice.
6. Dipping Infiltration.
   Pour resuspended Agrobacterium into 16 oz polypropylene containers.
   Invert 4-inch pots and submerge the aerial portion of the plants into the Agrobacterium suspension and let stand for 5 min.
   Pour out Agrobacterium suspension into waste bucket while keeping polypropylene container in place and return the plants to the upright position.
   Place 10 covered pots per flat.
   Fill each flat with 1-inch of water and cover with shade cloth.
   Keep covered for 24 hr and then remove shade cloth and polypropylene containers.
   Resume normal plant maintenance.
   When plants have finished flowering cover each pot with a ciber plant sleeve.
   After plants are completely dry, collect seed and place into 2.0 ml micro tubes and store in 100-place cryogenic boxes.
      Procedure: High Throughput Phenotypic Screening of Mutants—T1 Generation
1. Soil Preparation. Wear gloves at all times.
   In a large container, mix 60% autoclaved SunshineMix #5 with 40% vermiculite.
   Add 2.5 Tbsp of Osmocote, and 2.5 Tbsp of 1% granular Marathon per 25 L of soil.
   Mix thoroughly.
2. Fill Com-Packs with Soil.
   Loosely fill D601 Com-Packs level to the rim with the prepared soil.
   Place filled pot into utility flat with holes, within a no-hole utility flat.
   Repeat as necessary for planting. One flat set should contain 6 pots.
3. Saturate Soil.
   Evenly water all pots until the soil is saturated and water is collecting in the bottom of the flats.
   After the soil is completely saturated, dump out the excess water.
4. Plant the Seed.
5. Stratify the Seeds.
   After sowing the seed for all the flats, place them into a dark 4° C. cooler.
   Keep the flats in the cooler for 2 nights for WS seed. Other ecotypes may take longer. This cold treatment will help promote uniform germination of the seed.
6. Remove Flats from Cooler and Cover with Shade Cloth. (Shade cloth is only needed in the greenhouse)
   After the appropriate time, remove the flats from the cooler and place onto growth racks or benches.
   Cover the entire set of flats with 55% shade cloth. The cloth is necessary to cut down the light intensity during the delicate germination period.
   The cloth and domes should remain on the flats until the cotyledons have fully expanded. This usually takes about 4-5 days under standard greenhouse conditions.
7. Remove 55% Shade Cloth and Propagation Domes.
   After the cotyledons have fully expanded, remove both the 55% shade cloth and propagation domes.
8. Spray Plants with Finale Mixture. Wear gloves and protective clothing at all times.
   Prepare working Finale mixture by mixing 3 ml concentrated Finale in 48 oz of water in the Poly-TEK sprayer.
   Completely and evenly spray plants with a fine mist of the Finale mixture.
   Repeat Finale spraying every 3-4 days until only transformants remain. (Approximately 3 applications are necessary.)
   When satisfied that only transformants remain, discontinue Finale spraying.
9. Screen Each Pot for Phenotypes.
   The phenotype is recognized, by observing seeds that do not germinate or seedlings which die before reaching mature plant stage.

PCR was used to amplify the cDNA insert in one randomly chosen T₁ plant. This PCR product was then sequenced to confirm that the correct insert was contained in the plants. The quality control process was performed as per standard protocol.

### Microarray Analysis

A major way that a cell controls its response to internal or external stimuli is by regulating the rate of transcription of specific genes. For example, the differentiation of cells during organogenensis into forms characteristic of the organ is associated with the selective activation and repression of large numbers of genes. Thus, specific organs, tissues and cells are functionally distinct due to the different populations of mRNAs and protein products they possess. Internal signals program the selective activation and repression programs. For example, internally synthesized hormones produce such signals. The level of hormone can be raised by increasing the level of transcription of genes encoding proteins concerned with hormone synthesis.

To measure how a cell reacts to internal and/or external stimuli, individual mRNA levels can be measured and used as an indicator for the extent of transcription of the gene. Cells can be exposed to a stimulus, and mRNA can be isolated and assayed at different time points after stimulation The mRNA from the stimulated cells can be compared to control cells that were not stimulated. The mRNA levels that are higher in the stimulated cell versus the control indicate a stimulus-specific response of the cell. The same is true of mRNA levels that are lower in stimulated cells versus the control condition.

Similar studies can be performed with cells taken from an organism with a defined mutation in their genome as compared with cells without the mutation. Altered mRNA levels in the mutated cells indicate how the mutation causes transcriptional changes. These transcriptional changes are associated with the phenotype that the mutated cells exhibit that is different from the phenotype exhibited by the control cells.

Applicants have utilized microarray techniques to measure the levels of mRNAs in cells from plants transformed with the polynucleotides of the invention. In general, transformants with the genes of the invention were grown to an appropriate stage, and tissue samples were prepared for the microarray differential expression analysis.

### Microarray Experimental Procedures and Results

Procedures

A summary of the parameters utilized for each of the differential expression analysis experiments is provided in TABLE 9.

1. Sample Tissue Preparation

Tissue samples for each of the expression analysis experiments were prepared as follows:

(a:) Roots

Seeds of Arabidopsis thaliana (Ws) were sterilized in full strength bleach for less than 5 min., washed more than 3 times in sterile distilled deionized water and plated on MS agar plates. The plates were placed at 4° C. for 3 nights and then placed vertically into a growth chamber having 16 hr light/8 hr dark cycles, 23° C., 70% relative humidity and ˜11,000 LUX. After 2 weeks, the roots were cut from the agar, flash frozen in liquid nitrogen and stored at −80° C.

(b) Rosette Leaves, Stems, and Siliques

Arabidopsis thaliana (Ws) seed was vernalized at 4° C. for 3 days before sowing in Metro-mix soil type 350. Flats were placed in a growth chamber having 16 hr light/8 hr dark, 80% relative humidity, 23° C. and 13,000 LUX for germination and growth. After 3 weeks, rosette leaves, stems, and siliques were harvested, flash frozen in liquid nitrogen and stored at −80° C. until use. After 4 weeks, siliques (<5 mm, 5-10 mm and >10 mm) were harvested, flash frozen in liquid nitrogen and stored at −80° C. until use. 5 week old whole plants (used as controls) were harvested, flash frozen in liquid nitrogen and kept at −80° C. until RNA was isolated.

(c) Germination

Arabidopsis thaliana seeds (ecotype Ws) were sterilized in bleach and rinsed with sterile water. The seeds were placed in 100 mm petri plates containing soaked autoclaved filter paper. Plates were foil-wrapped and left at 4° C. for 3 nights to vernalize. After cold treatment, the foil was removed and plates were placed into a growth chamber having 16 hr light/8 hr dark cycles, 23° C., 70% relative humidity and ˜11,000 lux. Seeds were collected 1 d, 2 d, 3 d and 4 d later, flash frozen in liquid nitrogen and stored at −80° C. until RNA was isolated.

(d) Abscissic Acid (ABA)

Seeds of Arabidopsis thaliana (ecotype Wassilewskija) were sown in trays and left at 4° C. for 4 days to vernalize. They were then transferred to a growth chamber having grown 16 hr light/8 hr dark, 13,000 LUX 70% humidity, and 20° C. and watered twice a week with 1 L of 1× Hoagland's solution Approximately 1,000 14 day old plants were spayed with 200-250 mls of 100 μM ABA in a 0.02% solution of the detergent Silwet L-77. Whole seedlings, including roots, were harvested within a 15 to 20 minute time period at 1 hr and 6 hr after treatment, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 100 μM ABA for treatment. Control plants were treated with water. After 6 hr and 24 hr, aerial and root tissues were separated and flash frozen in liquid nitrogen prior to storage at −80° C.

(e) Brassinosteroid Responsive

Two separate experiments were performed, one with epi-brassinolide and one with the brassinosteroid biosynthetic inhibitor brassinazole. In the epi-brassinolide experiments, seeds of wild-type Arabidopsis thaliana (ecotype Wassilewskija) and the brassinosteroid biosynthetic mutant dwf4-1 were sown in trays and left at 4° C. for 4 days to vernalize. They were then transferred to a growth chamber having 16 hr light/8 hr dark, 11,000 LUX, 70% humidity and 22° C. temperature. Four week old plants were spayed with a 1 μM solution of epi-brassinolide and shoot parts (unopened floral primordia and shoot apical meristems) harvested three hours later. Tissue was flash-frozen in liquid nitrogen and stored at −80° C. In the brassinazole experiments, seeds of wild-type Arabidopsis thaliana (ecotype Wassilewskija) were grown as described above. Four week old plants were spayed with a 1 μM solution of brassinazole and shoot parts (unopened floral primordia and shoot apical meristems) harvested three hours later. Tissue was flash-frozen in liquid nitrogen and stored at −80° C.

In addition to the spray experiments, tissue was prepared from two different mutants; (1) a dwf4-1 knock out mutant and (2) a mutant overexpressing the dwf4-1 gene.

Seeds of wild-type Arabidopsis thaliana (ecotype Wassilewskija) and of the dwf4-1 knock out and overexpressor mutants were sown in trays and left at 4° C. for 4 days to vernalize. They were then transferred to a growth chamber having 16 hr light/8 hr dark, 11,000 LUX, 70% humidity and 22° C. temperature. Tissue from shoot parts (unopened floral primordia and shoot apical meristems) was flash-frozen in liquid nitrogen and stored at −80° C.

Another experiment was completed with seeds of Arabidopsis thaliana (ecotype Wassilewskija) were sown in trays and left at 4° C. for 4 days to vernalize. They were then transferred to a growth chamber. Plants were grown under long-day (16 hr light:8 hr. dark) conditions, 13,000 LUX light intensity, 70% humidity, 20° C. temperature and watered twice a week with 1 L 1× Hoagland's solution (recipe recited in Feldmann et al., (1987) Mol. Gen. Genet. 208: 1-9 and described as complete nutrient solution). Approximately 1,000 14 day old plants were spayed with 200-250 mls of 0.1 μM Epi-Brassinolite in 0.02% solution of the detergent Silwet L-77. At 1 hr. and 6 hrs. after treatment aerial tissues were harvested within a 15 to 20 minute time period and flash-frozen in liquid nitrogen.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 0.1 μM epi-brassinolide for treatment. Control plants were treated with distilled deionized water. After 24 hr, aerial and root tissues were separated and flash frozen in liquid nitrogen prior to storage at −80° C.

(f) Nitrogen: High to Low

Wild type Arabidopsis thaliana seeds (ecotpye Ws) were surface sterilized with 30% Clorox, 0.1% Triton X-100for 5 minutes. Seeds were then rinsed with 4-5 exchanges of sterile double distilled deionized water. Seeds were vernalized at 4° C. for 2-4 days in darkness. After cold treatment, seeds were plated on modified 1×MS media (without NH₄NO₃ or KNO₃), 0.5% sucrose, 0.5 g/L MES pH5.7, 1% phytagar and supplemented with KNO₃ to a final concentration of 60 mM (high nitrate modified 1×MS media). Plates were then grown for 7 days in a Percival growth chamber at 22° C. with 16 hr. light/8 hr dark.

Germinated seedlings were then transferred to a sterile flask containing 50 mL of high nitrate modified 1×MS liquid media. Seedlings were grown with mild shaking for 3 additional days at 22° C. in 16 hr. light/8 hr dark (in a Percival growth chamber) on the high nitrate modified 1×MS liquid media

After three days of growth on high nitrate modified 1×MS liquid media, seedlings were transferred either to a new sterile flask containing 50 mL of high nitrate modified 1×MS liquid media or to low nitrate modified 1×MS liquid media (containing 20 □M KNO₃). Seedlings were grown in these media conditions with mild shaking at 22° C. in 16 hr light/8 hr dark for the appropriate time points and whole seedlings harvested for total RNA isolation via the Trizol method (LifeTech). The time points used for the microarray experiments were 10 min. and 1 hour time points for both the high and low nitrate modified 1×MS media.

Alternatively, seeds that were surface sterilized in 30% bleach containing 0.1% Triton X-100 and further rinsed in sterile water, were planted on MS agar, (0.5% sucrose) plates containing 50 mM KNO₃ (potassium nitrate). The seedlings were grown under constant light (3500 LUX) at 22° C. After 12 days, seedlings were transferred to MS agar plates containing either 1 mM KNO₃ or 50 mM KNO₃. Seedlings transferred to agar plates containing 50 mM KNO₃ were treated as controls in the experiment. Seedlings transferred to plates with 1 mM KNO₃ were rinsed thoroughly with sterile MS solution containing 1 mM KNO₃. There were ten plates per transfer. Root tissue was collected and frozen in 15 mL Falcon tubes at various time points which included 1 hour, 2 hours, 3 hours, 4 hours, 6 hours, 9 hours, 12 hours, 16 hours, and 24 hours.

Maize 35A19 Pioneer hybrid seeds were sown on flats containing sand and grown in a Conviron growth chamber at 25° C., 16 hr light/8 hr dark, ˜13,000 LUX and 80% relative humidity. Plants were watered every three days with double distilled deionized water. Germinated seedlings are allowed to grow for 10 days and were watered with high nitrate modified 1×MS liquid media (see above). On day 11, young corn seedlings were removed from the sand (with their roots intact) and rinsed briefly in high nitrate modified 1×MS liquid media. The equivalent of half a flat of seedlings were then submerged (up to their roots) in a beaker containing either 500 mL of high or low nitrate modified 1×MS liquid media (see above for details).

At appropriate time points, seedlings were removed from their respective liquid media, the roots separated from the shoots and each tissue type flash frozen in liquid nitrogen and stored at −80° C. This was repeated for each time point. Total RNA was isolated using the Trizol method (see above) with root tissues only.

Corn root tissues isolated at the 4 hr and 16 hr time points were used for the microarray experiments. Both the high and low nitrate modified 1×MS media were used.

(g) Nitrogen: Low to High

Arabidopsis thaliana ecotype Ws seeds were sown on flats containing 4 L of a 1:2 mixture of Grace Zonolite vermiculite and soil. Flats were watered with 3 L of water and vernalized at 4° C. for five days. Flats were placed in a Conviron growth chamber having 16 hr light/8 hr dark at 20° C., 80% humidity and 17,450 LUX. Flats were watered with approximately 1.5 L of water every four days. Mature, bolting plants (24 days after germination) were bottom treated with 2 L of either a control (100 mM mannitol pH 5.5) or an experimental (50 mM ammonium nitrate, pH 5.5) solution. Roots, leaves and siliques were harvested separately 30, 120 and 240 minutes after treatment, flash frozen in liquid nitrogen and stored at −80° C.

Hybrid maize seed (Pioneer hybrid 35A19) were aerated overnight in deionized water. Thirty seeds were plated in each flat, which contained 4 liters of Grace zonolite vermiculite. Two liters of water were bottom fed and flats were kept in a Conviron growth chamber with 16 hr light/8 hr dark at 20° C. and 80% humidity. Flats were watered with 1 L of tap water every three days. Five day old seedlings were treated as described above with 2 L of either a control (100 mM mannitol pH 6.5) solution or 1 L of an experimental (50 mM ammonium nitrate, pH 6.8) solution. Fifteen shoots per time point per treatment were harvested 10, 90 and 180 minutes after treatment, flash frozen in liquid nitrogen and stored at −80° C.

Alternatively, seeds of Arabidopsis thaliana (ecotype Wassilewskija) were left at 4° C. for 3 days to vernalize. They were then sown on vermiculite in a growth chamber having 16 hours light/8 hours dark, 12,000-14,000 LUX, 70% humidity, and 20° C. They were bottom-watered with tap water, twice weekly. Twenty-four days old plants were sprayed with either water (control) or 0.6% ammonium nitrate at 4 μL/cm² of tray surface. Total shoots and some primary roots were cleaned of vermiculite, flash-frozen in liquid nitrogen and stored at −80° C.

(h) Methyl Jasmonate

Seeds of Arabidopsis thaliana (ecotype Wassilewskija) were sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1× Hoagland's solution. Approximately 1,000 14 day old plants were spayed with 200-250 mls of 0.001% methyl jasmonate in a 0.02% solution of the detergent Silwet L-77. At 1 hr and 6 hrs after treatment, whole seedlings, including roots, were harvested within a 15 to 20 minute time period, flash-frozen in liquid nitrogen and stored at −80° C.

Seeds of maize hybrid 35A (Pioneer) were sown in water moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 0.001% methyl jasmonate for treatment. Control plants were treated with water. After 24 hr, aerial and root tissues were separated and flash frozen in liquid nitrogen prior to storage at −80° C.

(i) Salicylic Acid

Seeds of Arabidopsis thaliana (ecotype Wassilewskija) were sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1× Hoagland's solution. Approximately 1,000 14 day old plants were spayed with 200-250 mls of 5 mM salicylic acid (solubilized in 70% ethanol) in a 0.02% solution of the detergent Silwet L-77. At 1 hr and 6 hrs after treatment, whole seedlings, including roots, were harvested within a 15 to 20 minute time period flash-frozen in liquid nitrogen and stored at −80° C.

Alternatively, seeds of wild-type Arabidopsis thaliana (ecotype Columbia) and mutant CS3726 were sown in soil type 200 mixed with osmocote fertilizer and Marathon insecticide and left at 4° C. for 3 days to vernalize. Flats were incubated at room temperature with continuous light. Sixteen days post germination plants were sprayed with 2 mM SA, 0.02% SilwettL-77 or control solution (0.02% SilwettL-77. Aerial parts or flowers were harvested 1 hr, 4 hr, 6 hr, 24 hr and 3 weeks post-treatment flash frozen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 2 mM SA for treatment. Control plants were treated with water. After 12 hr and 24 hr, aerial and root tissues were separated and flash frozen in liquid nitrogen prior to storage at −80° C.

(j) Drought Stress

Seeds of Arabidopsis thaliana (Wassilewskija) were sown in pots and left at 4° C. for three days to vernalize before being transferred to a growth chamber having 16 hr light/8 hr dark, 150,000-160,000 LUX, 20° C. and 70% humidity. After 14 days, aerial tissues were cut and left to dry on 3 MM Whatman paper in a petri-plate for 1 hour and 6 hours. Aerial tissues exposed for 1 hour and 6 hours to 3 MM Whatman paper wetted with 1× Hoagland's solution served as controls. Tissues were harvested, flash-frozen in liquid nitrogen and stored at −80° C.

Alternatively, Arabidopsis thaliana (Ws) seed was vernalized at 4° C. for 3 days before sowing in Metromix soil type 350. Flats were placed in a growth chamber with 23° C., 16 hr light/8 hr. dark, 80% relative humidity, ˜13,000 LUX for germination and growth. Plants were watered with 1-1.5 L of water every four days. Watering was stopped 16 days after germination for the treated samples, but continued for the control samples. Rosette leaves and stems, flowers and siliques were harvested 2 d, 3 d, 4 d, 5 d, 6 d and 7 d after watering was stopped. Tissue was flash frozen in liquid nitrogen and kept at −80° C. until RNA was isolated. Flowers and siliques were also harvested on day 8 from plants that had undergone a 7 d drought treatment followed by 1 day of watering. Control plants (whole plants) were harvested after 5 weeks, flash frozen in liquid nitrogen and stored as above.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in empty 1-liter beakers at room temperature for treatment. Control plants were placed in water. After 1 hr, 6 hr, 12 hr and 24 hr aerial and root tissues were separated and flash frozen in liquid nitrogen prior to storage at −80° C.

(k) Osmotic Stress

Seeds of Arabidopsis thaliana (Wassilewskija) were sown in trays and left at 4° C. for three days to vernalize before being transferred to a growth chamber having 16 hr light/8 hr dark, 12,000-14,000 LUX, 20° C., and 70% humidity. After 14 days, the aerial tissues were cut and placed on 3 MM Whatman paper in a petri-plate wetted with 20% PEG (polyethylene glycol-Mᵣ8,000) in 1× Hoagland's solution. Aerial tissues on 3 MM Whatman paper containing 1× Hoagland's solution alone served as the control. Aerial tissues were harvested at 1 hour and 6 hours after treatment, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 10% PEG (polyethylene glycol-Mᵣ8,000) for treatment. Control plants were treated with water. After 1 hr and 6 hr aerial and root tissues were separated and flash frozen in liquid nitrogen prior to storage at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered fiats were watered every three days for 7 days. Seedlings were carefully removed from the sand and placed in 1-liter beakers with 150 mM NaCl for treatment. Control plants were treated with water. After 1 hr, 6 hr, and 24 hr aerial and root tissues were separated and flash frozen in liquid nitrogen prior to storage at −80° C.

(l) Heat Shock Treatment

Seeds of Arabidopsis Thaliana (Wassilewskija) were sown in trays and left at 4° C. for three days to vernalize before being transferred to a growth chamber with 16 hr light/8 hr dark, 12,000-14,000 Lux, 70% humidity and 20° C., fourteen day old plants were transferred to a 42° C. growth chamber and aerial tissues were harvested 1 hr and 6 hr after transfer. Control plants were left at 20° C. and aerial tissues were harvested. Tissues were flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers containing 42° C. water for treatment. Control plants were treated with water at 25° C. After 1 hr and 6 hr aerial and root tissues were separated and flash frozen in liquid nitrogen prior to storage at −80° C.

(m) Cold Shock Treatment

Seeds of Arabidopsis thaliana (Wassilewskija) were sown in trays and left at 4° C. for three days to vernalize before being transferred to a growth chamber having 16 hr light/8 hr dark, 12,000-14,000 LUX, 20° C. and 70% humidity. Fourteen day old plants were transferred to a 4° C. dark growth chamber and aerial tissues were harvested 1 hour and 6 hours later. Control plants were maintained at 20° C. and covered with foil to avoid exposure to light. Tissues were flash-frozen in liquid nitrogen and stored at −80° C.

Seeds of maze hybrid 35A Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers containing 4° C. water for treatment. Control plants were treated with water at 25° C. After 1 hr and 6 hr aerial and root tissues were separated and flash frozen in liquid nitrogen prior to storage at −80° C.

(n) Arabidopsis Seeds

Fruits (pod+seed) 0-5 mm

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination, were summarized by Bowman (1994). Silique lengths were then determined and used as an approximate determinant for embryonic stage. Siliques 0-5 mm in length containing post fertilization through pre-heart stage [0-72 hours after fertilization (HAF)] embryos were harvested and flash frozen in liquid nitrogen.

Fruits (pod+seed) 5-10 mm

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Siliques 5-10 mm in length containing heart-through early upturned-U-stage [72-120 hours after fertilization (HAF)] embryos were harvested and flash frozen in liquid nitrogen.

Fruits (pod+seed) >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Siliques >10 mm in length containing green, late upturned-U-stage [>120 hours after fertilization (HAF)-9 days after flowering (DAF)] embryos were harvested and flash frozen in liquid nitrogen.

Green Pods 5-10 mm (Control Tissue for Samples 72-74)

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Green siliques 5-10 mm in length containing developing seeds 72-120 hours after fertilization (HAF)] were opened and the seeds removed. The remaining tissues (green pods minus seed) were harvested and flash frozen in liquid nitrogen.

Green Seeds from Fruits >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Green siliques>10 mm in length containing developing seeds up to 9 days after flowering (DAF)] were opened and the seeds removed and harvested and flash frozen in liquid nitrogen.

Brown Seeds from Fruits >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Yellowing siliques >10 mm in length containing brown, dessicating seeds >11 days after flowering (DAF)] were opened and the seeds removed and harvested and flash frozen in liquid nitrogen.

Green/Brown Seeds from Fruits >10 mm

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature. 3-4 siliques (fruits) bearing developing seeds were selected from at least 3 plants and were hand-dissected to determine what developmental stage(s) were represented by the enclosed embryos. Description of the stages of Arabidopsis embryogenesis used in this determination were summarized by Bowman (1994). Silique lengths were then determined and used as an approximate determinant for embryonic stage. Green siliques >10 mm in length containing both green and brown seeds >9 days after flowering (DAF)] were opened and the seeds removed and harvested and flash frozen in liquid nitrogen.

Mature Seeds (24 Hours after Imbibition)

Mature dry seeds of Arabidopsis thaliana (ecotype Wassilewskija) were sown onto moistened filter paper and left at 4° C. for two to three days to vernalize. Imbibed seeds were then transferred to a growth chamber [16 hr light:8 hr dark conditions, 7000-8000 LUX light intensity, 70% humidity, and 22° C. temperature], the emerging seedlings harvested after 48 hours and flash frozen in liquid nitrogen.

Mature Seeds (Dry)

Seeds of Arabidopsis thaliana (ecotype Wassilewskija) were sown in pots and left at 4° C. for two to three days to vernalize. They were then transferred to a growth chamber. Plants were grown under long-day (16 hr light:8 hr dark) conditions, 7000-8000 LUX light intensity, 70% humidity, and 22° C. temperature and taken to maturity. Mature dry seeds are collected, dried for one week at 28° C., and vernalized for one week at 4° C. before used as a source of RNA.

(o) Herbicide Treatment

Arabidopsis thaliana (Ws) seeds were sterilized for 5 min. with 30% bleach, 50 μl Triton in a total volume of 50 ml. Seeds were vernalized at 4° C. for 3 days before being plated onto GM agar plates at a density of about 144 seeds per plate. Plates were incubated in a Percival growth chamber having 16 hr light/8 hr dark, 80% relative humidity, 22° C. and 11,000 LUX for 14 days.

Plates were sprayed (˜0.5 mls/plate) with water, Finale (1.128 g/L), Glean (1.88 g/L), RoundUp (0.01 g/L) or Trimec (0.08 g/L). Tissue was collected and flash frozen in liquid nitrogen at the following time points: 0, 1, 2, 4, 8, 12 and 24 hours. Frozen tissue was stored at −80° C. prior to RNA isolation.

(p) Root Tips

Seeds of Arabidopsis thaliana (ecotye Ws) were placed on MS plates and vernalized at 4° C. for 3 days before being placed in a 25° C. growth chamber having 16 hr light/8 hr dark, 70% relative humidty and about 3 W/m². After 6 days, young seedlings were transferred to flasks containing B5 liquid medium, 1% sucrose and 0.05 mg/l indole-3-butyric acid. Flasks were incubated at room temperature with 100 rpm agitation. Media was replaced weekly. After three weeks, roots were harvested and incubated for 1 hr with 2% pectinase, 0.2% cellulose, pH 7 before straining through a #80 (Sigma) sieve. The root body material remaining on the sieve (used as the control) was flash frozen and stored at −80° C. until use. The material that passed through the #80 sieve was strained through a #200 (Sigma) sieve and the material remaining on the sieve (root tips) was flash frozen and stored at −80° C. until use. Approximately 10 mg of root tips were collected from one flask of root culture.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 8 days. Seedlings were carefully removed from the sand and the root tips (˜2 mm long) were removed and flash frozen in liquid nitrogen prior to storage at −80° C. The tissues above the root tips (˜1 cm long) were cut, treated as above and used as control tissue.

(q) Imbibed Seed

Seeds of maize hybrid 35A (Pioneer) were sown in water-moistened sand in covered flats (10 rows, 5-6 seed/row) and covered with clear, plastic lids before being placed in a growth chamber having 16 hr light (25° C.)/8 hr dark (20° C.), 75% relative humidity and 13,000-14,000 LUX. One day after sowing, whole seeds were flash frozen in liquid nitrogen prior to storage at −80° C. Two days after sowing, embryos and endosperm were isolated and flash frozen in liquid nitrogen prior to storage at −80° C. On days 3-6, aerial tissues, roots and endosperm were isolated and flash frozen in liquid nitrogen prior to storage at −80° C.

(r) Flowers (green, white or buds)

Approximately 10 μl of Arabidopsis thaliana seeds (ecotype Ws) were sown on 350 soil (containing 0.03% marathon) and vernalized at 4C. for 3 days. Plants were then grown at room temperature under fluorescent lighting until flowering. Flowers were harvested after 28 days in three different categories. Buds that had not opened at all and were completely green were categorized as “flower buds” (also referred to as green buds by the investigator). Buds that had started to open, with white petals emerging slightly were categorized as “green flowers” (also referred to as white buds by the investigator). Flowers that had opened mostly (with no silique elongation) with white petals completely visible were categorized as “white flowers” (also referred to as open flowers by the investigator). Buds and flowers were harvested with forceps, flash frozen in liquid nitrogen and stored at −80C. until RNA was isolated.

s) Ovules

Seeds of Arabidopsis thaliana heterozygous for pistillata (pi) [ecotype Landsberg erecta (Ler)] were sown in pots and left at 4° C. for two to three days to vernalize. They were then transferred to a growth chamber. Plants were grown under long-day (16 hr light:8 hr dark) conditions, 7000-8000 LUX light intensity, 76% humidity, and 24° C. temperature. Inflorescences were harvested from seedlings about 40 days old. The inflorescences were cut into small pieces and incubated in the following enzyme solution (pH 5) at room temperature for 0.5-1 hr.: 0.2% pectolyase Y-23, 0.04% pectinase, 5 mM MES, 3% Sucrose and MS salts (1900 mg/l KNO₃, 1650 mg/l NH₄NO₃, 370 mg/l MgSO₄.7 H₂O, 170 mg/l KH₂PO₄, 440 mg/l CaCl₂.2 H₂O, 6.2 mg/l H₂BO₃, 15.6 mg/l MnSO₄.4 H₂O, 8.6 mg/l ZnSO₄.7 H₂O, 0.25 mg/l NaMoO₄.2 H₂O, 0.025 mg/l CuCO₄.5 H₂O, 0.025 mg/l CoCl₂.6 H₂O, 0.83 mg/l KI, 27.8 mg/l FeSO₄.7 H₂O, 37.3 mg/l Disodium EDTA, pH 5.8). At the end of the incubation the mixture of inflorescence material and enzyme solution was passed through a size 60 sieve and then through a sieve with a pore size of 125 μm. Ovules greater than 125 μm in diameter were collected, rinsed twice in B5 liquid medium (2500 mg/l KNO_{3,} 250 mg/l MgSO₄.7 H₂O, 150 mg/l NaH2PO4.H₂O, 150 mg/l CaCl₂.2 H₂O, 134 mg/l (NH4)2 CaCl₂.SO₄, 3 mg/l H₂BO₃, 10 mg/l MnSO₄.4 H₂O, 2 ZnSO₄.7 H₂O, 0.25 mg/l NaMoO₄.2 H₂O, 0.025 mg/l CuCO₄.5 H₂O, 0.025 mg/l CoCl₂.6 H₂O, 0.75 mg/l KI, 40 mg/l EDTA sodium ferric salt, 20 g/l sucrose, 10 mg/l Thiamine hydrochloride, 1 mg/l Pyridoxine hydrochloride, 1 mg/l Nicotinic acid, 100 mg/l myo-inositol, pH 5.5)), rinsed once in deionized water and flash frozen in liquid nitrogen. The supernatant from the 125 μm sieving was passed through subsequent sieves of 50 μm and 32 μm. The tissue retained in the 32 μm sieve was collected and mRNA prepared for use as a control.

t) Wounding

Seeds of Arabidopsis thaliana (Wassilewskija) were sown in trays and left at 4° C. for three days to vernalize before being transferred to a growth chamber having 16 hr light/8 hr dark, 12,000-14,000 LUX, 70% humidity and 20° C. After 14 days, the leaves were wounded with forceps. Aerial tissues were harvested 1 hour and 6 hours after wounding. Aerial tissues from unwounded plants served as controls. Tissues were flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were wounded (one leaf nicked by scissors) and placed in 1-liter beakers of water for treatment. Control plants were treated not wounded. After 1 hr and 6 hr aerial and root tissues were separated and flash frozen in liquid nitrogen prior to storage at −80° C.

u) Nitric Oxide Treatment

Seeds of Arabidopsis thaliana (Wassilewskija) were sown in trays and left at 4° C. for three days to vernalize before being transferred to a growth chamber having 16 hr light/8 hr dark, 12,000-14,000 LUX, 20° C. and 70% humidity. Fourteen day old plants were sprayed with 5 mM sodium nitroprusside in a 0.02% Silwett L-77 solution. Control plants were sprayed with a 0.02% Silwett L-77 solution. Aerial tissues were harvested 1 hour and 6 hours after spraying, flash-frozen in liquid nitrogen and stored at −80° C.

Seeds of maize hybrid 35A (Pioneer) were sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings were carefully removed from the sand and placed in 1-liter beakers with 5 mM nitroprusside for treatment. Control plants were treated with water. After 1 hr, 6 hr and 12 hr, aerial and root tissues were separated and flash frozen in liquid nitrogen prior to storage at −80° C.

v) Root Hairless Mutants

Plants mutant at the rhl gene locus lack root hairs. This mutation is maintained as a heterozygote.

Seeds of Arabidopsis thaliana (Landsberg erecta) mutated at the rhl gene locus were sterilized using 30% bleach with 1 ul/ml 20% Triton-X 100 and then vernalized at 4° C. for 3 days before being plated onto GM agar plates. Plates were placed in growth chamber with 16 hr light/8 hr. dark, 23° C., 14,500-15,900 LUX, and 70% relative humidity for germination and growth.

After 7 days, seedlings were inspected for root hairs using a dissecting microscope. Mutants were harvested and the cotyledons removed so that only root tissue remained. Tissue was then flash frozen in liquid nitrogen and stored at −80C.

Arabidopsis thaliana (Landsberg erecta) seedlings grown and prepared as above were used as controls.

Alternatively, seeds of Arabidopsis thaliana (Landsberg erecta), heterozygous for the rhl1 (root hairless) mutation, were surface-sterilized in 30% bleach containing 0.1% Triton X-100 and further rinsed in sterile water. They were then vernalized at 4° C. for 4 days before being plated onto MS agar plates. The plates were maintained in a growth chamber at 24° C. with 16 hr light/8 hr dark for germination and growth. After 10 days, seedling roots that expressed the phenotype (i.e. lacking root hairs) were cut below the hypocotyl junction, frozen in liquid nitrogen and stored at −80° C. Those seedlings with the normal root phenotype (heterozygous or wt) were collected as described for the mutant and used as controls.

w) Ap2

Seeds of Arabidopsis thaliana (ecotype Landesberg erecta) and floral mutant apetala2 (Jofuku et al., 1994, Plant Cell 6:1211-1225) were sown in pots and left at 4° C. for two to three days to vernalize. They were then transferred to a growth chamber. Plants were grown under long-day (16 hr light, 8 hr dark) conditions 7000-8000 LUX light intensity, 70% humidity and 22° C. temperature. Inflorescences containing immature floral buds (stages 1-7; Bowman, 1994) as wel as the inflorescence meristem were harvested and flashfrozen. Polysomal polyA+ RNA was isolated from tissue according to Cox and Goldberg, 1988).

2. Microarray Hybridization Procedures

Microarray technology provides the ability to monitor mRNA transcript levels of thousands of genes in a single experiment. These experiments simultaneously hybridize two differentially labeled fluorescent cDNA pools to glass slides that have been previously spotted with cDNA clones of the same species. Each arrayed cDNA spot will have a corresponding ratio of fluorescence that represents the level of disparity between the respective mRNA species in the two sample pools. Thousands of polynucleotides can be spotted on one slide, and each experiment generates a global expression pattern.

Coating Slides

The microarray consists of a chemically coated microscope slide, referred herein as a “chip” with numerous polynucleotide samples arrayed at a high density. The poly-L-lysine coating allows for this spotting at high density by providing a hydrophobic surface, reducing the spreading of spots of DNA solution arrayed on the slides. Glass microscope slides (Gold Seal #3010 manufactured by Gold Seal Products, Portsmouth, N.H., USA) were coated with a 0.1 W/V solution of Poly-L-lysine (Sigma, St Louis, Mo.) using the following protocol:
1. Slides were placed in slide racks (Shandon Lipshaw #121). The racks were then put in chambers (Shandon Lipshaw #121).
2. Cleaning solution was prepared:
   70 g NaOH was dissolved in 280 mL ddH2O.
   420 mL 95% ethanol was added. The total volume was 700 mL (=2×350 mL); it was stirred until completely mixed. If the solution remained cloudy, ddH₂O was added until clear.
3. The solution was poured into chambers with slides; the chambers were covered with glass lids. The solution was mixed on an orbital shaker for 2 hr.
4. The racks were quickly transferred to fresh chambers filled with ddH₂O. They were rinsed vigorously by plunging racks up and down. Rinses were repeated 4× with fresh ddH₂O each time, to remove all traces of NaOH-ethanol.
5. Polylysine solution was prepared:
   0 mL poly-L-lysine+70 mL tissue culture PBS in 560 mL water, using plastic graduated cylinder and beaker.
6. Slides were transferred to polylysine solution and shaken for 1 hr.
7. The rack was transferred to a fresh chambers filled with ddH₂O. It was plunged up and down 5× to rinse.
8. The slides were centrifuged on microtiter plate carriers (paper towels were placed below the rack to absorb liquid) for 5 min. @ 500 rpm. The slide racks were transferred to empty chambers with covers.
9. Slide racks were dried in a 45C. oven for 10 min.
10. The slides were stored in a closed plastic slide box.
11. Normally, the surface of lysine coated slides was not very hydrophobic immediately after this process, but became increasingly hydrophobic with storage. A hydrophobic surface helped ensure that spots didn't run together while printing at high densities. After they aged for 10 days to a month the slides were ready to use. However, coated slides that have been sitting around for long periods of time were usually too old to be used. This was because they developed opaque patches, visible when held to the light, and these resulted in high background hybridization from the fluorescent probe. Alternatively, pre-coated glass slides were purchased from TeleChem International, Inc. (Sunnyvale, Calif., 94089; catalog number SMM-25, Superamine substrates).
   PCR Amplification of cDNA Clone Inserts.

Polynucleotides were amplified from Arabidopsis cDNA clones using insert specific probes. The resulting 100 uL PCR reactions were purified with Qiaquick 96 PCR purification columns (Qiagen, Valencia, Calif., USA) and eluted in 30 uL of 5 mM Tris. 8.5 uL of the elution were mixed with 1.5 uL of 20×SSC to give a final spotting solution of DNA in 3×SSC. The concentrations of DNA generated from each clone varied between 10-100 ng/ul, but were usually about 50 ng/ul.

### Arraying of PCR Products on Glass Slides

PCR products from cDNA clones were spotted onto the poly-L-Lysine coated glass slides using an arrangement of quill-tip pins (ChipMaker 3 spotting pins; Telechem, International, Inc., Sunnyvale, Calif., USA) and a robotic arrayer (PixSys 3500, Cartesian Technologies, Irvine, Calif., USA). Around 0.5 nl of a prepared PCR product was spotted at each location to produce spots with approximately 100 um diameters. Spot center-to-center spacing was from 180 um to 210 um depending on the array. Printing was conducted in a chamber with relative humidity set at 50%.

Slides containing maize sequences were purchased from Agilent Technology (Palo Alto, Calif. 94304).

### Post-Processing of Slides

After arraying, slides were processed through a series of steps—rehydration, UV cross-linking, blocking and denaturation—required prior to hybridization. Slides were rehydrated by placing them over a beaker of warn water (DNA face down), for 2-3 sec, to distribute the DNA more evenly within the spots, and then snap dried on a hot plate (DNA side, face up). The DNA was then cross-linked to the slides by UV irradiation (60-65 mJ; 2400 Stratalinker, Stratagene, La Jolla, Calif., USA).

Following this a blocking step was performed to modify remaining free lysine groups, and hence minimize their ability to bind labeled probe DNA. To achieve this the arrays were placed in a slide rack. An empty slide chamber was left ready on an orbital shaker. The rack was bent slightly inwards in the middle, to ensure the slides would not run into each other while shaking. The blocking solution was prepared as follows:
3×350-ml glass chambers (with metal tops) were set to one side, and a large round Pyrex dish with dH₂O was placed ready in the microwave. At this time, 15 ml sodium borate was prepared in a 50 ml conical tube.

6-g succinic anhydride was dissolved in approx. 325-350 mL 1-methyl-2-pyrrolidinone. Rapid addition of reagent was crucial.

a. Immediately after the last flake of the succinic anhydride dissolved, the 15-mL sodium borate was added.

b. Immediately after the sodium borate solution mixed in, the solution was poured into an empty slide chamber.

c. The slide rack was plunged rapidly and evenly in the solution. It was vigorously shaken up and down for a few seconds, making sure slides never left the solution.

d. It was mixed on an orbital shaker for 15-20 min. Meanwhile, the water in the Pyrex dish (enough to cover slide rack) was heated to boiling.

Following this, the slide rack was gently plunge in the 95C. water (just stopped boiling) for 2 min. Then the slide rack was plunged 5× in 95% ethanol. The slides and rack were centrifuged for 5 min. @ 500 rpm. The slides were loaded quickly and evenly onto the carriers to avoid streaking. The arrays were used immediately or store in slide box.

The Hybridization process began with the isolation of mRNA from the two tissues (see “Isolation of total RNA” and “Isolation of mRNA”, below) in question followed by their conversion to single stranded cDNA (see “Generation of probes for hybridization”, below). The cDNA from each tissue was independently labeled with a different fluorescent dye and then both samples were pooled together. This final differentially labeled cDNA pool was then placed on a processed microarray and allowed to hybridize (see “Hybridization and wash conditions”, below).

Isolation of Total RNA

Approximately 1 g of plant tissue was ground in liquid nitrogen to a fine powder and transferred into a 50-ml centrifuge tube containing 10 ml of Trizol reagent. The tube was vigorously vortexed for 1 min and then incubated at room temperature for 10-20 min. on an orbital shaker at 220 rpm. Two ml of chloroform was added to the tube and the solution vortexed vigorously for at least 30-sec before again incubating at room temperature with shaking. The sample was then centrifuged at 12,000×g (10,000 rpm) for 15-20 min at 4° C. The aqueous layer was removed and mixed by inversion with 2.5 ml of 1.2M NaCl/0.8 M Sodium Citrate and 2.5 ml of isopropyl alcohol added. After a 10 min. incubation at room temperature, the sample was centrifuged at 12,000×g (10,000 rpm) for 15 min at 4° C. The pellet was washed with 70% ethanol, re-centrifuged at 8,000 rpm for 5 min and then air dried at room temperature for 10 min. The resulting total RNA was dissolved in either TE (10 mM Tris-HCl, 1 mM EDTA, pH 8.0) or DEPC (diethylpyrocarbonate) treated deionized water (RNAse-free water). For subsequent isolation of mRNA using the Qiagen kit, the total RNA pellet was dissolved in RNAse-free water.

Isolation of mRNA

mRNA was isolated using the Qiagen Oligotex mRNA Spin-Column protocol (Qiagen, Valencia, Calif.). Briefly, 500 μl OBB buffer (20 mM Tris-Cl, pH 7.5, 1 M NaCl, 2 mM EDTA, 0.2% SDS) was added to 500 μl of total RNA (0.5-0.75 mg) and mixed thoroughly. The sample was first incubated at 70° C. for 3 min, then at room temperature for 10 minutes and finally centrifuged for 2 min at 14,000-18,000×g. The pellet was resuspended in 400 μl OW2 buffer (10 mM Tris-Cl, pH 7.5, 150 mM NaCl, 1 mM EDTA) by vortexing, the resulting solution placed on a small spin column in a 1.5 ml RNase-free microcentrifuge tube and centrifuged for 1 min at 14,000-18,000×g. The spin column was transferred to a new 1.5 ml RNase-free microcentrifuge tube and washed with 400 μl of OW2 buffer. To release the isolated mRNA from the resin, the spin column was again transferred to a new RNase-free 1.5 ml microcentrifuge tube, 20-100 μl 70° C. OEB buffer (5 mM Tris-Cl, pH 7.5) added and the resin resuspended in the resulting solution via pipeting. The mRNA solution was collected after centrifuging for 1 min at 14,000-18,000×g.

Alternatively, mRNA was isolated using the Stratagene Poly(A) Quik mRNA Isolation Kit (Startagene, La Jolla, Calif.). Here, up to 0.5 mg of total RNA (maximum volume of 1 ml) was incubated at 65° C. for 5 minutes, snap cooled on ice and 0.1× volumes of 10× sample buffer (10 mM Tris-HCl (pH 7.5), 1 mM EDTA (pH 8.0) 5 M NaCl) added. The RNA sample was applied to a prepared push column and passed through the column at a rate of ˜1 drop every 2 sec. The solution collected was reapplied to the column and collected as above. 200 μl of high salt buffer (10 mM Tris-HCl (pH 7.5), 1 mM EDTA, 0.5 NaCl) was applied to the column and passed through the column at a rate of ˜1 drop every 2 sec. This step was repeated and followed by three low salt buffer (10 mM Tris-HCl (pH 7.5), 1 mM EDTA, 0.1 M NaCl) washes preformed in a similar manner. mRNA was eluted by applying to the column four separate 200 μl aliquots of elution buffer (10 mM Tris-HCl (pH 7.5), 1 mM EDTA) preheated to 65° C. Here, the elution buffer was passed through the column at a rate of 1 drop/sec. The resulting mRNA solution was precipitated by adding 0.1× volumes of 10× sample buffer, 2.5 volumes of ice-cold 100% ethanol, incubating overnight at −20° C. and centrifuging at 14,000-18,000×g for 20-30 min at 4° C. The pellet was washed with 70% ethanol and air dried for 10 min. at room temperature before resuspension in RNase-free deionized water.

### Preparation of Yeast Controls

Plasmid DNA was isolated from the following yeast clones using Qiagen filtered maxiprep kits (Qiagen, Valencia, Calif.): YAL022c(Fun26), YAL031c(Fun21), YBR032w, YDL131w, YDL182w, YDL194w, YDL196w, YDR050c and YDR116c. Plasmid DNA was linearized with either BsrBI (YAL022c(Fun26), YAL031c(Fun21), YDL131w, YDL182w, YDL194w, YDL196w, YDR050c) or AflIII (YBR032w, YDR116c) and isolated.

In Vitro Transcription of Yeast Clones

The following solution was incubated at 37° C. for 2 hours: 17 μl of isolated yeast insert DNA (1 μg), 20 μl 5× buffer, 10 μl 100 mM DTT, 2.5 μl (100 U) RNasin, 20 μl 2.5 mM (ea.) rNTPs, 2.7 μl (40 U) SP6 polymerase and 27.8 μl RNase-free deionized water. 2 μl (2 U) Ampli DNase I was added and the incubation continued for another 15 min. 10 μl 5M NH₄OAC and 100 μl phenol:chloroform:isoamyl alcohol (25:24:1) were added, the solution vortexed and then centrifuged to separate the phases. To precipitate the RNA, 250 μl ethanol was added and the solution incubated at −20° C. for at least one hour. The sample was then centrifuged for 20 min at 4° C. at 14,000-18,000×g, the pellet washed with 500 μl of 70% ethanol, air dried at room temperature for 10 min and resuspended in 100 μl of RNase-free deionized water. The precipitation procedure was then repeated.

Alternatively, after the two-hour incubation, the solution was extracted with phenol/chloroform once before adding 0.1 volume 3M sodium acetate and 2.5 volumes of 100% ethanol. The solution was centrifuged at 15,000 rpm, 4° C. for 20 minutes and the pellet resuspended in RNase-free deionized water. The DNase I treatment was carried out at 37° C. for 30 minutes using 2 U of Ampli DNase I in the following reaction condition: 50 mM Tris-HCl (pH 7.5), 10 mM MgCl₂. The DNase I reaction was then stopped with the addition of NH₄OAC and phenol:chloroform:isoamyl alcohol (25:24:1), and RNA isolated as described above.

0.15-2.5 ng of the in vitro transcript RNA from each yeast clone were added to each plant mRNA sample prior to labeling to serve as positive (internal) probe controls.

### Generation of Probes for Hybridization

Generation of Labeled Probes for Hybridization from First-Strand cDNA

Hybridization probes were generated from isolated mRNA using an Atlas™ Glass Fluorescent Labeling Kit (Clontech Laboratories, Inc., Palo Alto, Calif., USA). This entails a two step labeling procedure that first incorporates primary aliphatic amino groups during cDNA synthesis and then couples fluorescent dye to the cDNA by reaction with the amino functional groups. Briefly, 5 μg of oligo(dt)₁₈ primer d(TTTTTTTTTTTTTTTTTTV) was mixed with Poly A+mRNA (1.5-2 μg mRNA isolated using the Qiagen Oligotex mRNA Spin-Column protocol or the Stratagene Poly(A) Quik mRNA Isolation protocol (Stratagene, La Jolla, Calif., USA)) in a total volume of 25 μl. The sample was incubated in a thermocycler at 70° C. for 5 min, cooled to 48° C. and 10 μl of 5× cDNA Synthesis Buffer (kit supplied), 5 μl 10× dNTP mix (dATP, dCTP, dGTP, dTTP and aminoallyl-dUTP; kit supplied), 7.5 μl deionized water and 2.5 μl MMLV Reverse Transcriptase (500 U) added. The reaction was then incubated at 48° C. for 30 minutes followed by 1 hr incubation at 42° C. At the end of the incubation the reaction was heated to 70° C. for 10 min, cooled to 37° C. and 0.5 μl (5 U) RNase H added, before incubating for 15 min at 37° C. The solution was vortexed for 1 min after the addition of 0.5 μl 0.5 M EDTA and 5 μl of QuickClean Resin (kit supplied) then centrifuged at 14,000-18,000×g for 1 min. After removing the supernatant to a 0.45 μm spin filter (kit supplied), the sample was again centrifuged at 14,000-18,000×g for 1 min, and 5.5 μl 3 M sodium acetate and 137.5 μl of 100% ethanol added to the sample before incubating at −20° C. for at least 1 hr. The sample was then centrifuged at 14,000-18,000×g at 4° C. for 20 min, the resulting pellet washed with 500 μl 70% ethanol, air-dried at room temperature for 10 min and resuspended in 10 μl of 2× fluorescent labeling buffer (kit provided). 10 μl each of the fluorescent dyes Cy3 and Cy5 (Amersham Pharmacia (Piscataway, N.J., USA); prepared according to Atlas™ kit directions of Clontech) were added and the sample incubated in the dark at room temperature for 30 min.

The fluorescently labeled first strand cDNA was precipitated by adding 2 μl 3M sodium acetate and 50 μl 100% ethanol, incubated at −20° C. for at least 2 hrs, centrifuged at 14,000-18,000×g for 20 min, washed with 70% ethanol, air-dried for 10 min and dissolved in 100 μl of water.

Alternatively, 3-4 μg mRNA, 2.5 (˜8.9 ng of in vitro translated mRNA) μl yeast control and 3 μg oligo dTV (TTTTTTTTTTTTTTTTTT(A/C/G) were mixed in a total volume of 24.7 μl. The sample was incubated in a thermocycler at 70° C. for 10 min. before chilling on ice. To this, 8 μl of 5× first strand buffer (SuperScript II RNase H-Reverse Transcriptase kit from Invitrogen (Carlsbad, Calif. 92008); cat no. 18064022), 0.8° C. of aa-dUTP/dNTP mix (50×; 25 mM dATP, 25 mM dGTP, 25 mM dCTP, 15 mM dTTP, 10 mM aminoallyl-dUTP), 4 μl of 0.1 M DTT and 2.5 μl (500 units) of Superscript R.T.II enzyme (Stratagene) were added. The sample was incubated at 42° C. for 2 hours before a mixture of 10° C. of 1M NaOH and 10° C. of 0.5 M EDTA were added. After a 15 minute incubation at 65° C., 25 μl of 1 M Tris pH 7.4 was added. This was mixed with 450 μl of water in a Microcon 30 column before centrifugation at 11,000×g for 12 min. The column was washed twice with 450 μl (centrifugation at 11,000 g, 12 min.) before eluting the sample by inverting the Microcon column and centrifuging at 11,000×g for 20 seconds. Sample was dehydrated by centrifugation under vacuum and stored at −20° C.

Each reaction pellet was dissolved in 9 μl of 0.1 M carbonate buffer (0.1M sodium carbonate and sodium bicarbonate, pH=8.5-9) and 4.5 μl of this placed in two microfuge tubes. 4.5 μl of each dye (in DMSO) were added and the mixture incubated in the dark for 1 hour. 4.5 μl of 4 M hydroxylamine was added and again incubated in the dark for 15 minutes.

Regardless of the method used for probe generation, the probe was purified using a Qiagen PCR cleanup kit (Qiagen, Valencia, Calif., USA), and eluted with 100 ul EB (kit provided). The sample was loaded on a Microcon YM-30 (Millipore, Bedford, Mass., USA) spin column and concentrated to 4-5 ul in volume.

Probes for the maize microarrays were generated using the Fluorescent Linear Amplification Kit (cat No. G2556A) from Agilent Technologies (Palo Alto, Calif.).

### Hybridization and Wash Conditions

The following Hybridization and Washing Condition were developed:

Hybridization Conditions:

Labeled probe was heated at 95° C. for 3 min and chilled on ice. Then 25 □L of the hybridization buffer which was warmed at 42C. was added to the probe, mixing by pipeting, to give a final concentration of:
50% formamide
   4×SSC
   0.03% SDS
5× Denhardt's solution
0.1 μg/ml single-stranded salmon sperm DNA

The probe was kept at 42C. Prior to the hybridization, the probe was heated for 1 more min., added to the array, and then covered with a glass cover slip. Slides were placed in hybridization chambers (Telechem, Sunnyvale, Calif.) and incubated at 42° C. overnight.

Washing Conditions:

A. Slides were washed in 1×SSC+0.03% SDS solution at room temperature for 5 minutes,
B. Slides were washed in 0.2×SSC at room temperature for 5 minutes,
C. Slides were washed in 0.05×SSC at room temperature for 5 minutes.

After A, B, and C, slides were spun at 800×g for 2 min. to dry. They were then scanned.

Maize microarrays were hybridized according to the instructions included Fluorescent Linear Amplification Kit (cat No. G2556A) from Agilent Technologies (Palo Alto, Calif.).

### Scanning of Slides

The chips were scanned using a ScanArray 3000 or 5000 (General Scanning, Watertown, Mass., USA). The chips were scanned at 543 and 633 nm, at 10 um resolution to measure the intensity of the two fluorescent dyes incorporated into the samples hybridized to the chips.

### Data Extraction and Analysis

The images generated by scanning slides consisted of two 16-bit TIFF images representing the fluorescent emissions of the two samples at each arrayed spot. These images were then quantified and processed for expression analysis using the data extraction software Imagene™ (Biodiscovery, Los Angeles, Calif., USA). Imagene output was subsequently analyzed using the analysis program Genespring™ (Silicon Genetics, San Carlos, Calif., USA). In Genespring, the data was imported using median pixel intensity measurements derived from Imagene output. Background subtraction, ratio calculation and normalization were all conducted in Genespring. Normalization was achieved by breaking the data in to 32 groups, each of which represented one of the 32 pin printing regions on the microarray. Groups consist of 360 to 550 spots. Each group was independently normalized by setting the median of ratios to one and multiplying ratios by the appropriate factor.

Results

The MA_diff Table (TABLE 10) presents the results of the differential expression experiments for the mRNAs, as reported by their corresponding cDNA ID number, that were differentially transcribed under a particular set of conditions as compared to a control sample. The cDNA ID numbers correspond to those utilized in the Reference and Sequence Tables. Increases in mRNA abundance levels in experimental plants versus the controls are denoted with the plus sign (+). Likewise, reductions in mRNA abundance levels in the experimental plants are denoted with the minus (−) sign.

The Table is organized according to the clone number with each set of experimental conditions being denoted by the term “Expt Rep ID:” followed by a “short name”. Table 9 links each “short name” with a short description of the experiment and the parameters.

The sequences showing differential expression in a particular experiment (denoted by either a “+” or “−” in the Table) thereby shows utility for a function in a plant, and these functions/utilities are described in detail below, where the title of each section (i.e. a “utility section”) is correlated with the particular differential expression experiment in TABLE 9.

### Organ-Affecting Genes, Gene Components, Products (including Differentiation and Function)

Root Genes

The economic values of roots arise not only from harvested adventitious roots or tubers, but also from the ability of roots to funnel nutrients to support growth of all plants and increase their vegetative material, seeds, fruits, etc. Roots have four main functions. First, they anchor the plant in the soil. Second they facilitate and regulate the molecular signals and molecular traffic between the plant, soil, and soil fauna. Third, the root provides a plant with nutrients gained from the soil or growth medium. Fourth, they condition local soil chemical and physical properties.

Root genes are active or potentially active to a greater extent in roots than in most other organs of the plant. These genes and gene products can regulate many plant traits from yield to stress tolerance. Root genes can be used to modulate root growth and development.

Differential Expression of the Sequences in Roots

The relative levels of mRNA product in the root versus the aerial portion of the plant was measured. Specifically, mRNA was isolated from roots and root tips of Arabidopsis plants and compared to mRNA isolated from the aerial portion of the plants utilizing microarray procedures.

Root Hair Genes, Gene Components and Products

Root hairs are specialized outgrowths of single epidermal cells termed trichoblasts. In many and perhaps all species of plants, the trichoblasts are regularly arranged around the perimeter of the root. In Arabidopsis, for example, trichoblasts tend to alternate with non-hair cells or atrichoblasts. This spatial patterning of the root epidermis is under genetic control, and a variety of mutants have been isolated in which this spacing is altered or in which root hairs are completely absent.

The root hair development genes of the instant invention are useful to modulate one or more processes of root hair structure and/or function including (1) development; (2) interaction with the soil and soil contents; (3) uptake and transport in the plant; and (4) interaction with microorganisms.

1.) Development

The surface cells of roots can develop into single epidermal cells termed trichoblasts or root hairs. Some of the root hairs will persist for the life of the plant; others will gradually die back; some may cease to function due to external influences. These genes and gene products can be used to modulate root hair density or root hair growth, including rate, timing, direction, and size, for example. These genes and gene products can also be used to modulate cell properties such as cell size, cell division, rate and direction and number, cell elongation, cell differentiation, lignified cell walls, epidermal cells (including trichoblasts) and root apical meristem cells (growth and initiation); and root hair architecture such as leaf cells under the trichome, cells forming the base of the trichome, trichome cells, and root hair responses. In addition these genes and gene products can be used to modulate one or more of the growth and development processes in response to internal plant programs or environmental stimuli in, for example, the seminal system, nodal system, hormone responses, Auxin, root cap abscission, root senescence, gravitropism, coordination of root growth and development with that of other organs (including leaves, flowers, seeds, fruits, and stems), and changes in soil environment (including water, minerals, Ph, and microfauna and flora).

2.) Interaction with Soil and Soil Contents

Root hairs are sites of intense chemical and biological activity and as a result can strongly modify the soil they contact. Roots hairs can be coated with surfactants and mucilage to facilitate these activities. Specifically, roots hairs are responsible for nutrient uptake by mobilizing and assimilating water, reluctant ions, organic and inorganic compounds and chemicals. In addition, they attract and interact with beneficial microfauna and flora. Root hairs also help to mitigate the effects of toxic ions, pathogens and stress. Thus, root hair genes and gene products can be used to modulate traits such as root hair surfactant and mucilage (including composition and secretion rate and time); nutrient uptake (including water, nitrate and other sources of nitrogen, phosphate, potassium, and micronutrients (e.g. iron, copper, etc.); microbe and nematode associations (such as bacteria including nitrogen-fixing bacteria, mycorrhizae, nodule-forming and other nematodes, and nitrogen fixation); oxygen transpiration; detoxification effects of iron, aluminum, cadium, mercury, salt and other soil constituents; pathogens (including chemical repellents) glucosinolates (GSL1), which release pathogen-controlling isothiocyanates; and changes in soil (such as Ph, mineral excess and depletion), and rhizosheath.

3.) Transport of Materials in Plants

Uptake of the nutrients by the root and root hairs contributes a source-sink effect in a plant. The greater source of nutrients, the more sinks, such as stems, leaves, flowers, seeds, fruits, etc. can draw sustenance to grow. Thus, root hair development genes and gene products can be used to modulate the vigor and yield of the overall plant as well as distinct cells, organs, or tissues of a plant. The genes and gene products, therefore, can modulate plant nutrition, growth rate (such as whole plant, including height, flowering time, etc., seedling, coleoptile elongation, young leaves, stems, flowers, seeds and fruit) and yield, including biomass (fresh and dry weight during any time in plant life, including maturation and senescence), number of flowers, number of seeds, seed yield, number, size, weight and harvest index (content and composition, e.g. amino acid, jasmonate, oil, protein and starch) and fruit yield (number, size, weight, harvest index, and post harvest quality).

### Reproduction Genes, Gene Components and Products

Reproduction genes are defined as genes or components of genes capable of modulating any aspect of sexual reproduction from flowering time and inflorescence development to fertilization and finally seed and fruit development. These genes are of great economic interest as well as biological importance. The fruit and vegeTable industry grosses over $1 billion USD a year. The seed market, valued at approximately $15 billion USD annually, is even more lucrative.

Inflorescence and Floral Development Genes, Gene Components and Products

During reproductive growth the plant enters a program of floral development that culminates in fertilization, followed by the production of seeds. Senescence may or may not follow. The flower formation is a precondition for the sexual propagation of plants and is therefore essential for the propagation of plants that cannot be propagated vegetatively as well as for the formation of seeds and fruits. The point of time at which the merely vegetative growth of plants changes into flower formation is of vital importance for example in agriculture, horticulture and plant breeding. Also the number of flowers is often of economic importance, for example in the case of various useful plants (tomato, cucumber, zucchini, cotton etc.) with which an increased number of flowers may lead to an increased yield, or in the case of growing ornamental plants and cut flowers.

Flowering plants exhibit one of two types of inflorescence architecture: indeterminate, in which the inflorescence grows indefinitely, or determinate, in which a terminal flower is produced. Adult organs of flowering plants develop from groups of stem cells called meristems. The identity of a meristem is inferred from structures it produces: vegetative meristems give rise to roots and leaves, inflorescence meristems give rise to flower meristems, and flower meristems give rise to floral organs such as sepals and petals. Not only are meristems capable of generating new meristems of different identity, but their own identity can change during development. For example, a vegetative shoot meristem can be transformed into an inflorescence meristem upon floral induction, and in some species, the inflorescence meristem itself will eventually become a flower meristem. Despite the importance of meristem transitions in plant development, little is known about the underlying mechanisms.

Following germination, the shoot meristem produces a series of leaf meristems on its flanks. However, once floral induction has occurred, the shoot meristem switches to the production of flower meristems. Flower meristems produce floral organ primordia, which develop individually into sepals, petals, stamens or carpels. Thus, flower formation can be thought of as a series of distinct developmental steps, i.e. floral induction, the formation of flower primordia and the production of flower organs. Mutations disrupting each of the steps have been isolated in a variety of species, suggesting that a genetic hierarchy directs the flowering process (see for review, Weigel and Meyerowitz, In Molecular Basis of Morphogenesis (ed. M. Bernfield). 51st Annual Symposium of the Society for Developmental Biology, pp. 93-107, New York, 1993).

Expression of many reproduction genes and gene products is orchestrated by internal programs or the surrounding environment of a plant. These genes can be used to modulate traits such as fruit and seed yield

Seed and Fruit Development Genes, Gene Components and Products

The ovule is the primary female sexual reproductive organ of flowering plants. At maturity it contains the egg cell and one large central cell containing two polar nuclei encased by two integuments that, after fertilization, develops into the embryo, endosperm, and seed coat of the mature seed, respectively. As the ovule develops into the seed, the ovary matures into the fruit or silique. As such, seed and fruit development requires the orchestrated transcription of numerous polynucleotides, some of which are ubiquitous, others that are embryo-specific and still others that are expressed only in the endosperm, seed coat, or fruit. Such genes are termed fruit development responsive genes and can be used to modulate seed and fruit growth and development such as seed size, seed yield, seed composition and seed dormancy.

Differential Expression of the Sequences in Siliques, Inflorescences and Flowers

The relative levels of mRNA product in the siliques relative to the plant as a whole was measured.

Differential Expression of the Sequences in Hybrid Seed Development

The levels of mRNA product in the seeds relative to those in a leaf and floral stems was measured.

### Development Genes, Gene Components and Products

Imbibition and Germination Responsive Genes, Gene Components and Products

Seeds are a vital component of the world's diet. Cereal grains alone, which comprise ˜90% of all cultivated seeds, contribute up to half of the global per capita energy intake. The primary organ system for seed production in flowering plants is the ovule. At maturity, the ovule consists of a haploid female gametophyte or embryo sac surrounded by several layers of maternal tissue including the nucleus and the integuments. The embryo sac typically contains seven cells including the egg cell, two synergids, a large central cell containing two polar nuclei, and three antipodal cells. That pollination results in the fertilization of both egg and central cell. The fertilized egg develops into the embryo. The fertilized central cell develops into the endosperm. And the integuments mature into the seed coat. As the ovule develops into the seed, the ovary matures into the fruit or silique. Late in development, the developing seed ends a period of extensive biosynthetic and cellular activity and begins to desiccate to complete its development and enter a dormant, metabolically quiescent state. Seed dormancy is generally an undesirable characteristic in agricultural crops, where rapid germination and growth are required. However, some degree of dormancy is advantageous, at least during seed development. This is particularly true for cereal crops because it prevents germination of grains while still on the ear of the parent plant (preharvest sprouting), a phenomenon that results in major losses to the agricultural industry. Extensive domestication and breeding of crop species have ostensibly reduced the level of dormancy mechanisms present in the seeds of their wild ancestors, although under some adverse environmental conditions, dormancy may reappear. By contrast, weed seeds frequently mature with inherent dormancy mechanisms that allow some seeds to persist in the soil for many years before completing germination.

Germination commences with imbibition, the uptake of water by the dry seed, and the activation of the quiescent embryo and endosperm. The result is a burst of intense metabolic activity. At the cellular level, the genome is transformed from an inactive state to one of intense transcriptional activity. Stored lipids, carbohydrates and proteins are catabolized fueling seedling growth and development. DNA and organelles are repaired, replicated and begin functioning. Cell expansion and cell division are triggered. The shoot and root apical meristem are activated and begin growth and organogenesis. Schematic 4 summarizes some of the metabolic and cellular processes that occur during imbibition. Germination is complete when a part of the embryo, the radicle, extends to penetrate the structures that surround it. In Arabidopsis, seed germination takes place within twenty-four (24) hours after imbibition. As such, germination requires the rapid and orchestrated transcription of numerous polynucleotides. Germination is followed by expansion of the hypocotyl and opening of the cotyledons. Meristem development continues to promote root growth and shoot growth, which is followed by early leaf formation.

Imbibition and Germination Genes

Imbibition and germination includes those events that commence with the uptake of water by the quiescent dry seed and terminate with the expansion and elongation of the shoots and roots. The germination period exists from imbibition to when part of the embryo, usually the radicle, extends to penetrate the seed coat that surrounds it. Imbibition and germination genes are defined as genes, gene components and products capable of modulating one or more processes of imbibition and germination described above. They are useful to modulate many plant traits from early vigor to yield to stress tolerance.

Differential Expression of the Sequences in Germination Seeds and Imbibed Embryos

The levels of mRNA product in the seeds versus the plant as a whole was measured.

### Hormone Responsive Genes, Gene Components and Products

Abscissic Acid Responsive Genes, Gene Components and Products

Plant hormones are naturally occurring substances, effective in very small amounts, which act as signals to stimulate or inhibit growth or regulate developmental processes in plants. Abscisic acid (ABA) is a ubiquitous hormone in vascular plants that has been detected in every major organ or living tissue from the root to the apical bud. The major physiological responses affected by ABA are dormancy, stress stomatal closure, water uptake, abscission and senescence. In contrast to Auxins, cytokinins and gibberellins, which are principally growth promoters, ABA primarily acts as an inhibitor of growth and metabolic processes.

Changes in ABA concentration internally or in the surrounding environment in contact with a plant results in modulation of many genes and gene products. These genes and/or products are responsible for effects on traits such as plant vigor and seed yield.

While ABA responsive polynucleotides and gene products can act alone, combinations of these polynucleotides also affect growth and development. Useful combinations include different ABA responsive polynucleotides and/or gene products that have similar transcription profiles or similar biological activities, and members of the same or similar biochemical pathways. Whole pathways or segments of pathways are controlled by transcription factor proteins and proteins controlling the activity of signal transduction pathways. Therefore, manipulation of such protein levels is especially useful for altering phenotypes and biochemical activities of plants. In addition, the combination of an ABA responsive polynucleotide and/or gene product with another environmentally responsive polynucleotide is also useful because of the interactions that exist between hormone-regulated pathways, stress and defence induced pathways, nutritional pathways and development.

Differential Expression of the Sequences in ABA Treated Plants

The relative levels of mRNA product in plants treated with ABA versus controls treated with water were measured.

### Brassinosteroid Responsive Genes, Gene Components and Products

Plant hormones are naturally occuring substances, effective in very small amounts, which act as signals to stimulate or inhibit growth or regulate developmental processes in plants. Brassinosteroids (BRs) are the most recently discovered, and least studied, class of plant hormones. The major physiological response affected by BRs is the longitudinal growth of young tissue via cell elongation and possibly cell division. Consequently, disruptions in BR metabolism, perception and activity frequently result in a dwarf phenotype. In addition, because BRs are derived from the sterol metabolic pathway, any perturbations to the sterol pathway can affect the BR pathway. In the same way, perturbations in the BR pathway can have effects on the later part of the sterol pathway and thus the sterol composition of membranes.

Changes in BR concentration in the surrounding environment or in contact with a plant result in modulation of many genes and gene products. These genes and/or products are responsible for effects on traits such as plant biomass and seed yield. These genes were discovered and characterized from a much larger set of genes by experiments designed to find genes whose mRNA abundance changed in response to application of BRs to plants.

While BR responsive polynucleotides and gene products can act alone, combinations of these polynucleotides also affect growth and development. Useful combinations include different BR responsive polynucleotides and/or gene products that have similar transcription profiles or similar biological activities, and members of the same or functionally related biochemical pathways. Whole pathways or segments of pathways are controlled by transcription factors and proteins controlling the activity of signal transduction pathways. Therefore, manipulation of such protein levels is especially useful for altering phenotypes and biochemical activities of plants. In addition, the combination of a BR responsive polynucleotide and/or gene product with another environmentally responsive polynucleotide is useful because of the interactions that exist between hormone-regulated pathways, stress pathways, nutritional pathways and development. Here, in addition to polynucleotides having similar transcription profiles and/or biological activities, useful combinations include polynucleotides that may have different transcription profiles but which participate in common or overlapping pathways.

Differential Expression of the Sequences in Epi-Brassinolide or Brassinozole Plants

The relative levels of mRNA product in plants treated with either epi-brassinolide or brassinozole were measured.

### Metabolism Affecting Genes, Gene Components and Products

Nitrogen Responsive Genes, Gene Components and Products

Nitrogen is often the rate-limiting element in plant growth, and all field crops have a fundamental dependence on exogenous nitrogen sources. Nitrogenous fertilizer, which is usually supplied as ammonium nitrate, potassium nitrate, or urea, typically accounts for 40% of the costs associated with crops, such as corn and wheat in intensive agriculture. Increased efficiency of nitrogen use by plants should enable the production of higher yields with existing fertilizer inputs and/or enable existing yields of crops to be obtained with lower fertilizer input, or better yields on soils of poorer quality. Also, higher amounts of proteins in the crops could also be produced more cost-effectively. “Nitrogen responsive” genes and gene products can be used to alter or modulate plant growth and development.

Differential Expression of the Sequences in Whole Seedlings, Shoots and Roots

The relative levels of mRNA product in whole seedlings, shoots and roots treated with either high or low nitrogen media were compared to controls.

### Viability Genes, Gene Components and Products

Plants contain many proteins and pathways that when blocked or induced lead to cell, organ or whole plant death. Gene variants that influence these pathways can have profound effects on plant survival, vigor and performance. The critical pathways include those concerned with metabolism and development or protection against stresses, diseases and pests. They also include those involved in apoptosis and necrosis. Viability genes can be modulated to affect cell or plant death. Herbicides are, by definition, chemicals that cause death of tissues, organs and whole plants. The genes and pathways that are activated or inactivated by herbicides include those that cause cell death as well as those that function to provide protection.

Differential Expression of the Sequences in Herbicide Treated Plants and Herbicide Resistant Mutants

The relative levels of mRNA product in plants treated with heribicide and mutants resistant to heribicides were compared to control plants.

### Stress Responsive Genes, Gene Components and Products

Wounding Responsive Genes, Gene Components and Products

Plants are continuously subjected to various forms of wounding from physical attacks including the damage created by pathogens and pests, wind, and contact with other objects. Therefore, survival and agricultural yields depend on constraining the damage created by the wounding process and inducing defense mechanisms against future damage.

Plants have evolved complex systems to minimize and/or repair local damage and to minimize subsequent attacks by pathogens or pests or their effects. These involve stimulation of cell division and cell elongation to repair tissues, induction of programmed cell death to isolate the damage caused mechanically and by invading pests and pathogens, and induction of long-range signaling systems to induce protecting molecules, in case of future attack. The genetic and biochemical systems associated with responses to wounding are connected with those associated with other stresses such as pathogen attack and drought.

Wounding responsive genes and gene products can be used to alter or modulate traits such as growth rate; whole plant height, width, or flowering time; organ development (such as coleoptile elongation, young leaves, roots, lateral roots, tuber formation, flowers, fruit, and seeds); biomass; fresh and dry, weight during any time in plant life, such as at maturation; number of flowers; number of seeds; seed yield, number, size, weight, harvest index (such as content and composition, e.g., amino acid, nitrogen, oil, protein, and carbohydrate); fruit yield, number, size, weight, harvest index, post harvest quality, content and composition (e.g., amino acid, carotenoid, jasmonate, protein, and starch); seed and fruit development; germination of dormant and non-dormant seeds; seed viability, seed reserve mobilization, fruit ripening, initiation of the reproductive cycle from a vegetative state, flower development time, insect attraction for fertilization, time to fruit maturity, senescence; fruits, fruit drop; leaves; stress and disease responses; drought; heat and cold; wounding by any source, including wind, objects, pests and pathogens; uv and high light damage (insect, fungus, virus, worm, nematode damage).

Cold Responsive Genes Gene Components and Products

The ability to endure low temperatures and freezing is a major determinant of the geographical distribution and productivity of agricultural crops. Even in areas considered suiTable for the cultivation of a given species or cultivar, can give rise to yield decreases and crop failures as a result of aberrant, freezing temperatures. Even modest increases (1-2° C.) in the freezing tolerance of certain crop species would have a dramatic impact on agricultural productivity in some areas. The development of genotypes with increased freezing tolerance would provide a more reliable means to minimize crop losses and diminish the use of energy-costly practices to modify the microclimate.

Sudden cold temperatures result in modulation of many genes and gene products, including promoters. These genes and/or products are responsible for effects on traits such as plant vigor and seed yield.

Manipulation of one or more cold responsive gene activities is useful to modulate growth and development.

Differential Expression of the Sequences in Cold Treated Plants

The relative levels of mRNA product in cold treated plants were compared to control plants.

### Heat Responsive Genes, Gene Components and Products

The ability to endure high temperatures is a major determinant of the geographical distribution and productivity of agricultural crops. Decreases in yield and crop failure frequently occur as a result of aberrant, hot conditions even in areas considered suiTable for the cultivation of a given species or cultivar. Only modest increases in the heat tolerance of crop species would have a dramatic impact on agricultural productivity. The development of genotypes with increased heat tolerance would provide a more reliable means to minimize crop losses and diminish the use of energy-costly practices to modify the microclimate.

Changes in temperature in the surrounding environment or in a plant microclimate results in modulation of many genes and gene products.

Differential Expression of the Sequences in Heat Treated Plants

The relative levels of mRNA product in heat treated plants were compared to control plants.

### Drought Responsive Genes, Gene Components and Products

The ability to endure drought conditions is a major determinant of the geographical distribution and productivity of agricultural crops. Decreases in yield and crop failure frequently occur as a result of aberrant, drought conditions even in areas considered suiTable for the cultivation of a given species or cultivar. Only modest increases in the drought tolerance of crop species would have a dramatic impact on agricultural productivity. The development of genotypes with increased drought tolerance would provide a more reliable means to minimize crop losses and diminish the use of energy-costly practices to modify the microclimate.

Drought conditions in the surrounding environment or within a plant, results in modulation of many genes and gene products.

Differential Expression of the Sequences in Drought Treated Plants and Drought Mutants

The relative levels of mRNA product in drought treated plants and drought mutants were compared to control plants.

### Methyl Jasmonate (Jasmonate) Responsive Genes, Gene Components and Products

Jasmonic acid and its derivatives, collectively referred to as jasmonates, are naturally occurring derivatives of plant lipids. These substances are synthesized from linolenic acid in a lipoxygenase-dependent biosynthetic pathway. Jasmonates are signalling molecules which have been shown to be growth regulators as well as regulators of defense and stress responses. As such, jasmonates represent a separate class of plant hormones. Jasmonate responsive genes can be used to modulate plant growth and development.

Differential Expression of the Sequences in Methyl Jasmonate Treated Plants

The relative levels of mRNA product in methyl jasmonate treated plants were compared to control plants.

### Salicylic Acid Responsive Genes, Gene Components and Products

Plant defense responses can be divided into two groups: constitutive and induced. Salicylic acid (SA) is a signaling molecule necessary for activation of the plant induced defense system known as systemic acquired resistance or SAR. This response, which is triggered by prior exposure to avirulent pathogens, is long lasting and provides protection against a broad spectrum of pathogens. Another induced defense system is the hypersensitive response (HR). HR is far more rapid, occurs at the sites of pathogen (avirulent pathogens) entry and precedes SAR. SA is also the key signaling molecule for this defense pathway.

Differential Expression of the Sequences in Salicylic Acid Treated Plants

The relative levels of mRNA product in salicylic acid treated plants were compared to control plants.

### Osmotic Stress Responsive Genes, Gene Components and Products

The ability to endure and recover from osmotic and salt related stress is a major determinant of the geographical distribution and productivity of agricultural crops. Osmotic stress is a major component of stress imposed by saline soil and water deficit. Decreases in yield and crop failure frequently occur as a result of aberrant or transient environmental stress conditions even in areas considered suitable for the cultivation of a given species or cultivar. Only modest increases in the osmotic and salt tolerance of a crop species would have a dramatic impact on agricultural productivity. The development of genotypes with increased osmotic tolerance would provide a more reliable means to minimize crop losses and diminish the use of energy-costly practices to modify the soil environment. Thus, osmotic stress responsive genes can be used to modulate plant growth and development.

Differential Expression of the Sequences in PEG Treated Plants

The relative levels of mRNA product in PEG treated plants were compared to control plants.

### Shade Responsive Genes, Gene Components and Products

Plants sense the ratio of Red (R):Far Red (FR) light in their environment and respond differently to particular ratios. A low R:FR ratio, for example, enhances cell elongation and favors flowering over leaf production. The changes in R:FR ratios mimic and cause the shading response effects in plants. The response of a plant to shade in the canopy structures of agricultural crop fields influences crop yields significantly. Therefore manipulation of genes regulating the shade avoidance responses can improve crop yields. While phytochromes mediate the shade avoidance response, the down-stream factors participating in this pathway are largely unknown. One potential downstream participant, ATHB-2, is a member of the HD-Zip class of transcription factors and shows a strong and rapid response to changes in the R:FR ratio. ATHB-2 overexpressors have a thinner root mass, smaller and fewer leaves and longer hypocotyls and petioles. This elongation arises from longer epidermal and cortical cells, and a decrease in secondary vascular tissues, paralleling the changes observed in wild-type seedlings grown under conditions simulating canopy shade. On the other hand, plants with reduced ATHB-2 expression have a thick root mass and many larger leaves and shorter hypocotyls and petioles. Here, the changes in the hypocotyl result from shorter epidermal and cortical cells and increased proliferation of vascular tissue. Interestingly, application of Auxin is able to reverse the root phenotypic consequences of high ATHB-2 levels, restoring the wild-type phenotype. Consequently, given that ATHB-2 is tightly regulated by phytochrome, these data suggest that ATHB-2 may link the Auxin and phytochrome pathways in the shade avoidance response pathway.

Shade responsive genes can be used to modulate plant growth and development.

Differential Expression of the Sequences in Far-Red Light Treated Plants

The relative levels of mRNA product in far-red light treated plants were compared to control plants.

### Viability Genes, Gene Components and Products

Plants contain many proteins and pathways that when blocked or induced lead to cell, organ or whole plant death. Gene variants that influence these pathways can have profound effects on plant survival, vigor and performance. The critical pathways include those concerned with metabolism and development or protection against stresses, diseases and pests. They also include those involved in apoptosis and necrosis. The applicants have elucidated many such genes and pathways by discovering genes that when inactivated lead to cell or plant death.

Herbicides are, by definition, chemicals that cause death of tissues, organs and whole plants. The genes and pathways that are activated or inactivated by herbicides include those that cause cell death as well as those that function to provide protection. The applicants have elucidated these genes.

The genes defined in this section have many uses including manipulating which cells, tissues and organs are selectively killed, which are protected, making plants resistant to herbicides, discovering new herbicides and making plants resistant to various stresses.

Viability genes were also identified from a much larger set of genes by experiments designed to find genes whose mRNA products changed in concentration in response to applications of different herbicides to plants. Viability genes are characteristically differentially transcribed in response to fluctuating herbicide levels or concentrations, whether internal or external to an organism or cell. The MA_diff Table reports the changes in transcript levels of various viability genes.

### Early Seedling-Phase Specific Responsive Genes, Gene Components and Products

One of the more active stages of the plant life cycle is a few days after germination is complete, also referred to as the early seedling phase. During this period the plant begins development and growth of the first leaves, roots, and other organs not found in the embryo. Generally this stage begins when germination ends. The first sign that germination has been completed is usually that there is an increase in length and fresh weight of the radicle. Such genes and gene products can regulate a number of plant traits to modulate yield. For example, these genes are active or potentially active to a greater extent in developing and rapidly growing cells, tissues and organs, as exemplified by development and growth of a seedling 3 or 4 days after planting a seed.

Rapid, efficient establishment of a seedling is very important in commercial agriculture and horticulture. It is also vital that resources are approximately partitioned between shoot and root to facilitate adaptive growth. Phototropism and geotropism need to be established. All these require post-germination process to be sustained to ensure that vigorous seedlings are produced. Early seedling phase genes, gene components and products are useful to manipulate these and other processes.

### Guard Cell Genes, Gene Components and Products

Scattered throughout the epidermis of the shoot are minute pores called stomata. Each stomal pore is surrounded by two guard cells. The guard cells control the size of the stomal pore, which is critical since the stomata control the exchange of carbon dioxide, oxygen, and water vapor between the interior of the plant and the outside atmosphere. Stomata open and close through turgor changes driven by ion fluxes, which occur mainly through the guard cell plasma membrane and tonoplast. Guard cells are known to respond to a number of external stimuli such as changes in light intensity, carbon dioxide and water vapor, for example. Guard cells can also sense and rapidly respond to internal stimuli including changes in ABA, auxin and calcium ion flux.

Thus, genes, gene products, and fragments thereof differentially transcribed and/or translated in guard cells can be useful to modulate ABA responses, drought tolerance, respiration, water potential, and water management as examples. All of which can in turn affect plant yield including seed yield, harvest index, fruit yield, etc.

To identify such guard cell genes, gene products, and fragments thereof, Applicants have performed a microarray experiment comparing the transcript levels of genes in guard cells versus leaves. Experimental data is shown below.

### Nitric Oxide Responsive Genes, Gene Components and Products

The rate-limiting element in plant growth and yield is often its ability to tolerate suboptimal or stress conditions, including pathogen attack conditions, wounding and the presence of various other factors. To combat such conditions, plant cells deploy a battery of inducible defense responses, including synergistic interactions between nitric oxide (NO), reactive oxygen intermediates (ROS), and salicylic acid (SA). NO has been shown to play a critical role in the activation of innate immune and inflammatory responses in animals. At least part of this mammalian signaling pathway is present in plants, where NO is known to potentiate the hypersensitive response (HR). In addition, NO is a stimulator molecule in plant photomorphogenesis.

Changes in nitric oxide concentration in the internal or surrounding environment, or in contact with a plant, results in modulation of many genes and gene products.

In addition, the combination of a nitric oxide responsive polynucleotide and/or gene product with other environmentally responsive polynucleotides is also useful because of the interactions that exist between hormone regulated pathways, stress pathways, pathogen stimulated pathways, nutritional pathways and development.

Nitric oxide responsive genes and gene products can function either to increase or dampen the above phenotypes or activities either in response to changes in nitric oxide concentration or in the absence of nitric oxide fluctuations. More specifically, these genes and gene products can modulate stress responses in an organism. In plants, these genes and gene products are useful for modulating yield under stress conditions. Measurments of yield include seed yield, seed size, fruit yield, fruit size, etc.

### Shoot-Apical Meristem Genes, Gene Components and Products

New organs, stems, leaves, branches and inflorescences develop from the stem apical meristem (SAM). The growth structure and architecture of the plant therefore depends on the behavior of SAMs. Shoot apical meristems (SAMs) are comprised of a number of morphologically undifferentiated, dividing cells located at the tips of shoots. SAM genes elucidated here are capable of modifying the activity of SAMs and thereby many traits of economic interest from ornamental leaf shape to organ number to responses to plant density.

In addition, a key attribute of the SAM is its capacity for self-renewal. Thus, SAM genes of the instant invention are useful for modulating one or more processes of SAM structure and/or function including (I) cell size and division; (II) cell differentiation and organ primordia. The genes and gene components of this invention are useful for modulating any one or all of these cell division processes generally, as in timing and rate, for example. In addition, the polynucleotides and polypeptides of the invention can control the response of these processes to the internal plant programs associated with embryogenesis, and hormone responses, for example.

Because SAMs determine the architecture of the plant, modified plants will be useful in many agricultural, horticultural, forestry and other industrial sectors. Plants with a different shape, numbers of flowers and seed and fruits will have altered yields of plant parts. For example, plants with more branches can produce more flowers, seed or fruits. Trees without lateral branches will produce long lengths of clean timber. Plants with greater yields of specific plant parts will be useful sources of constituent chemicals.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

LENGTHY TABLE REFERENCED HERE
US20070101460A1-20070503-T00001
Please refer to the end of the specification for access instructions.

LENGTHY TABLE REFERENCED HERE
US20070101460A1-20070503-T00002
Please refer to the end of the specification for access instructions.

LENGTHY TABLE REFERENCED HERE
US20070101460A1-20070503-T00003
Please refer to the end of the specification for access instructions.

LENGTHY TABLE REFERENCED HERE
US20070101460A1-20070503-T00004
Please refer to the end of the specification for access instructions.

LENGTHY TABLE REFERENCED HERE
US20070101460A1-20070503-T00005
Please refer to the end of the specification for access instructions.

LENGTHY TABLE REFERENCED HERE
US20070101460A1-20070503-T00006
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070101460A1). An electronic copy of the table will also be available from the USPTO upon request and payment of the fee set forth in 37 CFR 1.19(b)(3).

What is claimed is:

1. An isolated nucleic acid molecule comprising:
a) a nucleic acid having a nucleotide sequence which encodes an amino acid sequence exhibiting at least 65% sequence identity to an amino acid sequence according to any one of the amino acid sequences set forth in the sequence listing;
b) a nucleic acid having a nucleotide sequence which encodes an amino acid sequence as set forth in the consensus sequence table;
c) a nucleic acid which is a complement of a nucleotide sequence according to paragraph (a) or (b);
d) a nucleic acid which is the reverse of the nucleotide sequence according to subparagraph (a) or (b), such that the reverse nucleotide sequence has a sequence order which is the reverse of the sequence order of the nucleotide sequence according to subparagraph (a); or
e) a nucleic acid capable of hybridizing to a nucleic acid according to any one of paragraphs (a)-(d), under conditions that permit formation of a nucleic acid duplex at a temperature from about 40° C. and 48° C. below the melting temperature of the nucleic acid duplex.

2. The isolated nucleic acid molecule according to claim 1, which has the sequence according to any one of the nucleotide sequences set forth in the sequence listing.

3. The isolated nucleic acid molecule according to claim 1, wherein said amino acid sequence comprises the amino acid sequence according to any one of the amino acid sequences set forth in the sequence listing or set forth in the consensus sequence table.

4. A vector construct comprising:
a) a first nucleic acid having a regulatory sequence capable of causing transcription and/or translation in a plant; and
b) a second nucleic acid having the sequence of the isolated nucleic acid molecule according to any one of claims 1-3;
wherein said first and second nucleic acids are operably linked and wherein said second nucleic acid is heterologous to any element in said vector construct.

5. The vector construct according to claim 4, wherein said first nucleic acid is native to said second nucleic acid.

6. The vector construct according to claim 4, wherein said first nucleic acid is heterologous to said second nucleic acid.

7. A host cell comprising an isolated nucleic acid molecule according to any one of claims 1-3, wherein said nucleic acid molecule is flanked by exogenous sequence.

8. A host cell comprising a vector construct according to any one of claims 4-6.

9. An isolated polypeptide comprising an amino acid sequence exhibiting at least 85% sequence identity of any one of the amino acid sequences set forth in the sequence listing, and having the characteristic of being a lethal or non-viability polypeptide.

10. A method of introducing an isolated nucleic acid into a host cell comprising:
a) providing an isolated nucleic acid molecule according to any one of claims 1-3; and
b) contacting said isolated nucleic with said host cell under conditions that permit insertion of said nucleic acid into said host cell.

11. A method of transforming a host cell which comprises contacting a host cell with a vector construct according to any one of claims 4-6.

12. A method of modulating the viability or germination characteristics of plant seed material comprising transforming said plant to over express a nucleic acid molecule according to claim 1 or a vector according to claim 4.

13. A method of producing a plant with non-viable or non-fertile seed comprising transforming said plant to over express a nucleic acid molecule according to any one of claims 1-4 or a vector according to any one of claims 4-6.

14. A method of producing plant seed material that is not viable or capable of regenerating into a mature plant comprising transforming said plant with a nucleic acid molecule according to any one of claims 1-3 or a vector according to any one of claims 4-6.

15. A method for producing a plant with seed material this is not fertile, comprising transforming a plant with a nucleic acid molecule that codes for a polypeptide according to any one of the amino acid sequences set forth in the sequence listing or in the consensus sequence table.

16. A method for detecting a nucleic acid in a sample which comprises:
a) providing an isolated nucleic acid molecule according to any one of claims 1-3;
b) contacting said isolated nucleic acid molecule with a sample under conditions which permit a comparison of the sequence of said isolated nucleic acid molecule with the sequence of DNA in said sample; and
c) analyzing the result of said comparison.

17. A plant, plant cell, plant material or seed of a plant which comprises a nucleic acid molecule according to any one of claims 1-3 which is exogenous or heterologous to said plant or plant cell.

18. A plant, plant cell, plant material or seed of a plant which comprises a vector construct according to any one of claims 4-6.

19. A plant which has been regenerated from a plant cell or seed according to claims 17 or 18.
